(19) United States
(12) Patent Application Publication
(10) Pub. No.: US 2005/0142135 A1
(43) Pub. Date: Jun. 30, 2005

(54) Agents and methods for modulating interactions between gonadotropin hormones and receptors

(75) Inventors: Tae H. Ji, Lexington, KY (US); Inhae Ji, Lexington, KY (US)

Correspondence Address: BURNS DOANE SWECKER & MATHIS L L P, POST OFFICE BOX 1404, ALEXANDRIA, VA, 22313-1404, US

(73) Assignee: University of Kentucky Research Foundation, Lexington, KY (US)

(21) Appl. No.: 10/821,939

(22) Filed: Apr. 12, 2004

Related U.S. Application Data
(60) Provisional application No. 60/461,836, filed on Apr. 11, 2003.

(51) Int. Cl. A61K 39/395; C07K 16/26

(52) U.S. Cl. 424/145.1; 530/388.24

(57) ABSTRACT

The present invention relates to agents and methods for the modulation of gonadotropin hormones and their receptors, including methods of treating gonadotropin disorders and conditions and screening and development of therapies. Specifically, the present invention relates to modulation of gonadotropin hormones through the inhibition of activity of exoloop 1, exoloop 2 and exoloop 3 of gonadotropin receptors.

### IDENTIFICATION OF FEDERAL FUNDING

The present invention was supported by Grants HD-18702 and DK-51469 from the National Institutes of Health, and therefore the government may have rights in the invention.

### FIELD OF THE INVENTION

The present invention relates to agents and methods for the modulation of gonadotropin hormones and their receptors, including methods of treating gonadotropin disorders and conditions and screening and development of therapies. Specifically, the present invention relates to modulation of gonadotropin hormones through the inhibition of activity of exoloop 1, exoloop 2 and exoloop 3 of gonadotropin receptors.

### BACKGROUND OF THE INVENTION

Reproduction, normal growth and development, and the maintenance of metabolic responses are all necessary both for individuals and for the perpetuation of a species. All three of these processes are affected by hormones. The leutinizing hormone receptor (LHR) and the follicle-stimulating hormone receptor (FSHR) both play crucial roles in the reproduction and development of species. Each year millions of people suffer from hormone related disorders-such as infertility, impotence and certain types of cancer because something has gone wrong with the interaction between these receptors and/or the hormones that they bind.

The hormones leutinizing hormone (LH) and follicle stimulating hormone (FSH), as well as the closely related placental hormone, chorionic gonadotropin (CG), are referred to as the gonadotropin hormones because of their actions on gonadal cells.

The binding of hormone to the LH/hCG or FSH receptors causes an activation of the G protein, stimulation of adenylyl cyclase activity, an increase in intracellular cyclic AMP levels (cAMP), and an associated increase in cAMP dependent protein kinase activity. This response occurs when only a very small fraction of cell surface receptors are occupied by hormone. At much higher concentrations of hormone, when a larger fraction of cell surface receptors are occupied, the gonadotropin receptors also cause a stimulation of phospholipase C activity, resulting in an increase of the breakdown of polyphosphatidylinositol phosphates, an increase in intracellular Ca²⁺, and an increase in protein kinase C activity. Human chorionic gonadotropin (hCG) is produced by the syncytiotrophoblast, the epithelium surrounding the fetus. The major biological function of hCG is to stimulate the production of progesterone from cholesterol by the corpus luteum. This ensures a continual supply of ovarian progesterone. hCG is a heterodimeric glycoprotein of 57 kDa consisting of a noncovalently bound α subunit (92 amino acids) and a separate β subunit (134 amino acids). There is one gene for the α subunit located on chromosome 6. Chromosome 19 contains a cluster of 6 genes coding for the hCG β subunit. The structures of the hCG and LH β subunits are very similar, with 80% identity.

LH and FSH are synthesized and secreted by gonadotrope cells in the anterior pituitary. The actions of LH and CG are mediated by the leutinizing hormone receptor (LHR) (also known as the LH/hCG receptor), and the actions of FSH are mediated by the FSH receptor (FSHR). The isolation and expression of cDNAs for the LH/hCG and FSH receptors has revealed that these receptors are each single polypeptides of approximately 700 amino acids, and possess a seven transmembrane spanning domain characteristic of the receptor family of G protein-coupled receptors. However, unlike most other members of this superfamily, the gonadotropin receptors also contain a large amino-terminal glucosylated extracellular domain which contains about half of the molecule. Truncated versions of the gonadotropin receptors that represent only the amino-terminal extracellular domains bind hormone with high affinity, suggesting that the extracellular domain (exodomain) is responsible for conferring binding specificity and high-affinity binding. See Norman et al., 1997 (Hormones, 2d edition, Academic Press, 138-140).

FSH is a heterodimeric hormone consisting of a 15 kDa glycosylated α subunit and a 18 kDa glycosylated β subunit. The dimeric structure is important for high affinity receptor binding and activation. The two subunits are tightly but noncovalently associated, and a discrete region with a concave surface, is thought to interact with FSHR. The FSH receptor belongs to a subfamily of glycoprotein hormone receptors within the G protein coupled receptor family. It comprises two halves of ˜350 amino acids, the extracellular N-terminal exodomain and membrane associated c- terminal endodomain that includes 7 transmembrane helices. The exodomain binds the hormone with high affinity without hormone action. The exodomain/hormone complex undergoes a conformational change, and is thought to modulate the endodomain, thus generating a signal. Indeed, the entire extracellular domain undergoes a conformational change when introduced into a membrane mimicking detergent. Therefore, the high affinity interaction of the exodomain and FSH is the crucial first step leading to signal generation and hormone action. Despite the importance of this initial binding event, only limited information is available concerning the precise contact residues and sites in the exodomain as well as the hormone.

The FSH receptor (FSHR) and other glycoprotein hormone (LH/CG and TSH) receptors belong to a structurally unique subfamily of G protein-coupled receptors. They comprise two equal halves, an N-terminal extracellular half (exodomain) and a C-terminal membrane associated half (endodomain). The exodomain is ˜350 amino acids long and alone is capable of high affinity hormone binding with hormone selectivity but without hormone action. Receptor activation occurs in the endodomain which is structurally equivalent to the entire molecule of many other G protein-coupled receptors. Glycoprotein hormones initially bind to the exodomain, and then the resulting hormone/exodomain complex modulates the endodomain, which activates adenylyl cyclase (AC) to generate cAMP and phospholipase Cβ (PLCβ) to produce inositol phosphate and diacylglycerol. Therefore, the ternary interactions among the hormone, exodomain and endodomain are crucial for successful signal generation. It has been reported that FSH and hCG binding to their cognate receptors is regulated by certain residues of exoloops 2 and 3 of the endodomain. Furthermore, the hinge region of the exodomain interacts with exoloop 2 and modulates cAMP induction. These results suggest that the exodomain interacts with the exoloops and modulates them for signal generation.

Glycoprotein hormones initially bind to the exodomain, and the resulting hormone/exodomain complex modulates the endodomain. The ternary interactions among the hormone, exodomain and endodomain are crucial for activation of adenylyl cyclase to generate cAMP and phospholipase Cβ to produce inositol phosphate and diacylglycerol. Despite the crucial roles of the ternary interactions, its nature has not been well defined. In the case of the LH receptor, the hinge region and Leucine Rich Repeat 4 of the exodomain interact with the endodomain. The hinge residues are involved in pairing with exoloop 2 and suppressing the receptor activation. A discussion of the role of the Leucine Rich Repeats in gonadotropin hormone binding, as well as a discussion of the gonadotropins in the diagnosis and treatment of hormone related conditions may be found in U.S. patent application Ser. No. 10/187,176, filed on Jul. 2, 2002 (which claims priority from U.S. Provisional Application No. 60/301,834, filed Jul. 2, 2001), which is incorporated by reference in its entirety herein.

In contrast, the Leu Rich Repeat 4 residues appear to promote the activation of the endodomain, but the contact site is unknown. Some residues of exoloop 3 in the endodomain modulate the hormone binding to the exodomain. These observations suggest that the exoloops be likely involved in interactions between the exodomain and endodomain.

Thus, the present invention addresses the interaction of the exoloops with the hormone. Specifically, the present invention examines exoloops 1-3 of LHR and FSHR. Exoloop 3 consists of 11 amino acids, connects the transmembrane domains 6 and 7 that are important for activation of AC, and has been implicated in the cAMP signal generation. Exoloop 3 of LHR interacts with both subunits of hCG, whereas FSHR exoloop 3 contacts with the α subunit of FSH as reported recently.

Thus, there is a need in the art for compositions and methods of modulating the action of gonadotropins and their receptors which can be used to effectively treat gonadotropin related problems with great efficacy and without the associated side effects.

The gonadotropin hormones and their receptors are known in the art. For example, Moyle et al., WO 92/22667, disclose analogs of glycoprotein hormone receptors which bind CG, LH and FSH, as well as the methods of preparing same. The structure of the LHR gene is disclosed by Atger et al., 1995 (Molecular and Cellular Endocrinology, 111:113-123), along with the LHR gene promoter. Igarashi et al., JP 405271285, disclose a LHR protein, which can be produced by culturing a transformant integrated with a DNA containing a cDNA coding the human LHR protein. Nikolics et al., WO 90/13643, disclose the purification and cloning of gonadotropin receptors. Kelton et al., U.S. Pat. No. 6,121,016 disclose an essentially pure human FSH receptor, or fragment thereof, which can bind to FSH, as well as the DNA encoding an expression vector for same.

However, previously, the mechanism by which the hormones bind the receptor was not understood. This invention discloses that the mechanism by which hormones bind the gondadotropin receptors has been discovered. This interaction involves novel exoloops of the gondadotropin receptors, which has been unexpectedly discovered to be a domain where the gondadotropins bind. This interaction has been found to trigger hormone signal.

### SUMMARY OF THE INVENTION

The present invention relates to the novel interaction between exoloops 1, 2 and 3 of the LH/CG and FSH receptors with the CG and FSH. This interaction has unexpectedly been found to trigger hormone signaling.

In one embodiment, the present invention is directed to a method of modulating the interaction between CG and the LHR in a subject comprising administering a therapeutically effective amount of an agent which modulates a CG activity, wherein the agent modulates CG activity by binding to the exoloop 1, exoloop 2 or exoloop 3 of the LHR or to the binding domain of CG creating an agent/exodomain or agent/CG complex. Preferably, the CG is hCG.

Preferably, the subject is a vertebrate or an invertebrate organism. The subject may be a canine, a feline, an ovine, a primate, an equine, a porcine, a caprine, a camelid, an avian, a bovine, an amphibian, a fish, or a murine organism. Preferably, the primate is a human. The human may be male or female.

The agent may be CG or a biologically active fragment thereof or any other natural or synthetic compound. The agent may block CG binding to the LHR by binding to the LHR or may block CG binding to the LHR by binding to the CG.

In a further embodiment, the present invention is directed to a method of regulating a CG activity in a subject comprising administering a therapeutically effective amount of an agent which modulates CG activity or modulates CG interaction with the LHR at the site of the exoloop 1, exoloop 2 or exoloop 3 on the LHR by modulating the interaction of an exoloop 1, exoloop 2 or exoloop 3 motif on the LHR with the CG.

In a further embodiment, the present invention is directed to a method of treating a gonadotropin hormone related disease or condition in a male subject comprising administering to the subject a therapeutically effective amount of an agent which modulates CG activity or CG interaction with the LHR at the site of the exoloop 1, exoloop 2 or exoloop 3 on the LHR. The gonadotropin hormone related disorder may be selected from a group consisting of male pseudohermaphroditism, microphallus, gynecomastia, bilateral anorchia, absence of Leydig's cells, cryptorchidism, Noonan's syndrome and myotonic dystrophy, delayed puberty, precocious puberty, acne and impotence.

In a further embodiment, the present invention is directed to a method of treating a gonadotropin hormone related disease in a female subject comprising administering to said subject a therapeutically effective amount of an agent which modulates CG activity or CG interaction with the LHR at the site of the exoloop 1, exoloop 2 or exoloop 3 on the LHR. The gonadotropin hormone related disorder may be selected from the group consisting of primary and secondary amenorrhea, delayed puberty, precocious puberty, endometriosis, acne, uterine myoma, ovarian and mammary cystic diseases, and breast and gynecological cancers.

In a further embodiment, the present invention is directed to a method of contraception in a subject comprising administering to a subject an amount of an agent effective at preventing conception, wherein the agent inhibits CG activity or CG interaction with the exoloop 1, exoloop 2 or exoloop 3 domain of the LHR. The agent may be a CG or biologically active fragment thereof or any other natural or synthetic compound. The subject may be male or female.

In a further embodiment, the present invention is directed to a method of promoting fertility in a subject comprising administering to a subject an amount of an agent effective at stimulating fertility, wherein the agent stimulates CG activity or CG interaction with the exoloop 1, exoloop 2 or exoloop 3 domain of the LHR. The agent may be a CG or biologically active fragment thereof or any other natural or synthetic compound.

In a further embodiment, the present invention is directed to a method of screening for compounds which modulate the interaction between CG and the exoloop 1, exoloop 2 or exoloop 3 domain on the LHR comprising: (a) attaching CG or a biologically active polypeptide fragment thereof to a substrate; (b) exposing CG or the biologically active polypeptide fragment thereof to an agent; and (c) determining whether said agent bound to CG or the biologically active polypeptide fragment thereof and further determining whether said agent modulates the interaction between CG and the exoloop 1, exoloop 2 or exoloop 3 domain of the LHR. The present invention also contemplates a compound indentified by this method.

In a further embodiment, the present invention is directed to a composition for treating gonadotropin hormone related diseases comprising a pharmaceutically effective amount of a compound which modulates the LHR at the exoloop 1, exoloop 2 or exoloop 3 domain and a pharmaceutically acceptable excipient. The compound may be CG or a biologically active fragment thereof. The LHR modulating compound may be an agent which binds to CG thereby preventing its interaction with the LHR at the site of the exoloop 1, exoloop 2 or exoloop 3 on the LHR.

In a further embodiment, the present invention is directed to a composition for treating a gonadotropin hormone related disease comprising a pharmaceutically acceptable amount of an agent which modulates CG activity, wherein the agent is an antibody which binds to CG and thereby prevents CG from interacting with LHR at the exoloop 1, exoloop 2 or exoloop 3 domain.

In a further embodiment, the present invention is directed to a method of modulating at least one activity of CG comprising administering an effective amount of an agent which modulates at least one activity of CG at the exoloop 1, exoloop 2 or exoloop 3 domain of the LHR. The modulated activity may be selected from the group consisting of stimulation of progesterone, androgen and estrogen and stimulation of development of the male and female gonads, follicles, placenta, maturation of oocytes and sperm and growth of cells and tissue.

In a further embodiment, the present invention is directed to a method of identifying binding partners for CG comprising the steps of: (a) exposing the protein to a potential binding partner; and (b) determining if an exoloop 1, exoloop 2 or exoloop 3 domain of the potential binding partner binds to CG.

In a further embodiment, the present invention is directed to a method of modulating the interaction between FSH and the FSHR in a subject comprising administering a therapeutically effective amount of an agent which modulates a FSH activity, wherein the agent modulates the FSH activity by binding to the exoloop 1, exoloop 2 or exoloop 3 of the FSHR or to FSH creating an agent/exodomain or agent/FSH complex. The subject may be a vertebrate or an invertebrate organism. The agent may be FSH or a biologically active fragment thereof or any other natural or synthetic compound. The agent may block FSH binding to the FSHR by binding to the FSHR or blocks FSH binding to the FSHR by binding to the FSH.

In a further embodiment, the present invention is directed to a method of regulating a FSH activity in a subject comprising administering a therapeutically effective amount of an agent which modulates FSH activity or modulates FSH interaction with the FSHR at the site of the exoloop 1, exoloop 2 or exoloop 3 on the FSHR by modulating the interaction of exoloop 1, exoloop 2 or exoloop 3 on the FSHR with the FSH.

In a further embodiment, the present invention is directed to a method of treating a gonadotropin hormone related disease or condition in a male subject comprising administering to the subject a therapeutically effective amount of an agent which modulates FSH activity or FSH interaction with the FSHR at the site of the exoloop 1, exoloop 2 or exoloop 3 on the FSHR. The gonadotropin hormone related disorder may be selected from a group consisting of male pseudohermaphroditism, microphallus, gynecomastia, bilateral anorchia, absence of Leydig's cells, cryptorchidism, Noonan's syndrome and myotonic dystrophy, delayed puberty, precocious puberty, acne and impotence.

In a further embodiment, the present invention is directed to a method of treating a gonadotropin hormone related disease in a female subject comprising administering to said subject a therapeutically effective amount of which modulates FSH activity or FSH interaction at the site of the exoloop 1, exoloop 2 or exoloop 3 on the FSHR. The gonadotropin hormone related disorder is selected from the group consisting of primary and secondary amenorrhea, delayed puberty, precocious puberty, endometriosis, acne, uterine myoma, ovarian and mammary cystic diseases, and breast and gynecological cancers.

In a further embodiment, the present invention is directed to a method of contraception in a subject comprising administering to a subject an amount of an agent effective at preventing conception, wherein the agent inhibits FSH activity or FSH interaction with the exoloop 1, exoloop 2 or exoloop 3 domain of the FSHR. The agent may be FSH or biologically active fragment thereof or any other natural or synthetic compound.

In a further embodiment, the present invention is directed to a method of promoting fertility in a subject comprising administering to a subject an amount of an agent effective at stimulating fertility, wherein the agent stimulates FSH activity or FSH interaction with the exoloop 1, exoloop 2 or exoloop 3 domain of the FSHR. The agent may be FSH or biologically active fragment thereof or any other natural or synthetic compound.

In a further embodiment, the present invention is directed to a method of screening for compounds which modulate the interaction between FSH and the exoloop 1, exoloop 2 or exoloop 3 domain on the FSHR comprising: (a) attaching FSH or a biologically active polypeptide fragment thereof to a substrate; (b) exposing FSH or the biologically active polypeptide fragment thereof to an agent; and (c) determining whether said agent bound to FSH or the biologically active polypeptide fragment thereof and further determining whether said agent modulates the interaction between FSH and the exoloop 1, exoloop 2 or exoloop 3 domain of the FSHR. The present invention also contemplates a compound identified by this method.

A composition for treating gonadotropin hormone related diseases comprising a pharmaceutically effective amount of a compound which modulates the FSHR at the exoloop 1, exoloop 2 or exoloop 3 domain and a pharmaceutically acceptable excipient. The compound which modulates the FSHR at the exoloop 1, exoloop 2 or exoloop 3 domain is FSH or a biologically active fragment thereof. The FSHR modulating compound may be an agent which binds to FSH thereby preventing its interaction with the FSHR at the site of exoloop 3 on the FSHR

In a further embodiment, the present invention is directed to a composition for treating a gonadotropin hormone related disease comprising a pharmaceutically acceptable amount of an agent which modulated FSH activity, wherein the agent is an antibody which binds to FSH and thereby prevents it from interacting with the FSHR at the exoloop 1, exoloop 2 or exoloop 3 domain.

In a further embodiment, the present invention is directed to a method of modulating at least one activity of FSH comprising administering an effective amount of an agent which modulates at least one activity of FSH at the exoloop 1, exoloop 2 or exoloop 3 domain of the FSHR. The modulated activity may be selected from the group consisting of stimulation of progesterone, androgen and estrogen and stimulation of development of the male and female gonads, follicles, placenta, maturation of oocytes and sperm and growth of cells and tissue.

In a further embodiment, the present invention is directed to a method of identifying binding partners for FSH comprising the steps of: (a) exposing the protein to a potential binding partner; and (b) determining if the exoloop 1, exoloop 2 or exoloop 3 domain of the potential binding partner binds to FSH.

### BRIEF DESCRIPTION OF THE DRAWINGS

FIG. 1 shows a comparison of the primary sequence of the first 34 residues of the glycoprotein hormone receptors. The FSH receptor sequences of various species were compared with the corresponding sequences of the human LH receptor and TSH receptor C¹⁵ of FSHR is conserved among the species.

FIG. 2 shows Ala substitutions for S⁹-V²⁰. Residues from S⁹ to V²⁰ of the FSH receptor were individually substituted with Ala and the resulting mutant receptors were stably expressed in HEK293 cells. Intact cells were used for ¹²⁵I-FSH binding in the presence of increasing concentrations of unlabeled FSH (A and D) and for cAMP production (C and F). The competitive inhibition data (A and D) were converted to Scatchard plot. Experiments were repeated several times in duplicate. NS stands for “not significant”.

FIG. 3 shows Ala substitutions for T²¹-E³³. Residues from T²¹ to E³³ of the FSH receptor were individually substituted with Ala, and the resulting mutant receptors were expressed in HEK293 cells and assayed.

FIG. 4 shows a comparison of Ala substitution mutations. To easily compare the activities of the wild type and mutant receptors, the ratios of Kd^{wild type/mutant}, EC₅₀^{wild type/mutant}, and maximum cAMP^{mutant/wild type} were presented in a bar graph.

FIG. 5 shows FSH binding in solution. Cells transfected with the C¹⁵A, P²⁴A, D²⁶A or L²⁷A mutant receptor were solubilized in NP-40 and assayed for FSH binding.

FIG. 6 shows photoaffinity labeling of FSH with photoactivable FSHR^{9-40F13Bpa}. The FSH receptor peptide corresponding to the sequence S9-K40, FSHR⁹⁻⁴⁰, was synthesized with a Tyr at the N-terminus for radioiodination and Bpa at the position of F¹³ for photoaffinity labeling (A). The peptide was radioiodinated and the resulting ¹²⁵I-FSHR^{9-40F13Bpa} was incubated with FSH and irradiated with UV. (B) The sample was irradiated with UV for increasing time periods from 0 to 150 s, solubilized in SDS under the reducing condition, and electrophoresed on polyacrylamide gel. After drying gels they were exposed to a phosphoimaging screen and scanned on a phosphoimager. The peptide appeared as the lower band, and the FSHα and FSH β subunits comigrated and appeared in the upper band. (C) Increasing amounts of ¹²⁵I-FSHR^{9-40F13Bpa} from 0 to 3.7 μM were incubated with a constant amount (0.1 μM) of FSH and photolyzed for 60 s. The samples were processed as described above. (D) Increasing amounts of FSH from 0 to 0.2 μM were incubated with a constant amount (3.1 μM) of ¹²⁵I-FSHR^{9-40F13Bpa}.

FIG. 7 shows inhibition of photoaffinity labeling and ¹²⁵I-FSH binding to FSHR. Constant amounts of ¹²⁵I-FSHR^{9-40F13Bpa} and FSH were incubated in the presence of increasing concentrations of non-radioactive peptides, FSHR⁹⁻⁴⁰ (A) or FSHR^{9-40F13Bpa} (B), treated with UV, and processed as described in the legend to FIG. 6. (C) A constant amount of ¹²⁵I-FSHR^{9-40F13Bpa} was incubated with 10 nM of FSH, phospholipase A (PLA), urokinase or growth hormone (QH), LH or TSH, treated with UV, and processed.

FIG. 8 shows photoaffinity labeling of denatured FSH. (A) Increasing concentrations of ¹²⁵I-FSHR^{9-40F13Bpa} were incubated with 80 nM of denatured FSH, irradiated with UV, and processed as described in the legend to FIG. 6. FSH was denatured by boiling in 8 M urea for 30 min. (B) A constant amount of ¹²⁵I-FSHR^{9-40F13Bpa} was incubated with increasing concentrations of denatured FSH, treated with UV, and processed.

FIG. 9 shows an immunoblot of FSH α and β subunit bands. (A) A constant amount of FSH was incubated with increasing concentrations of FSHR treated with UV, deglycosylated with PNGase F, solubilized, and electrophoresed along with ¹²⁵I-FSH. (B) FSH was treated with PNGase F, solubilized in SDS under the reducing condition, and electrophoresed along with non-deglycosylated FSH. Gel lanes were either stained with commassie brilliant blue (CBB) or blotted and stained with antiFSH, antiFSH α or antiFSH β antibodies.

FIG. 10 shows sequence alignment of exoloop 3. The exoloop 3 sequences of FSHR, LHR and TSHR were aligned among species. Identical residues are presented as “-”.

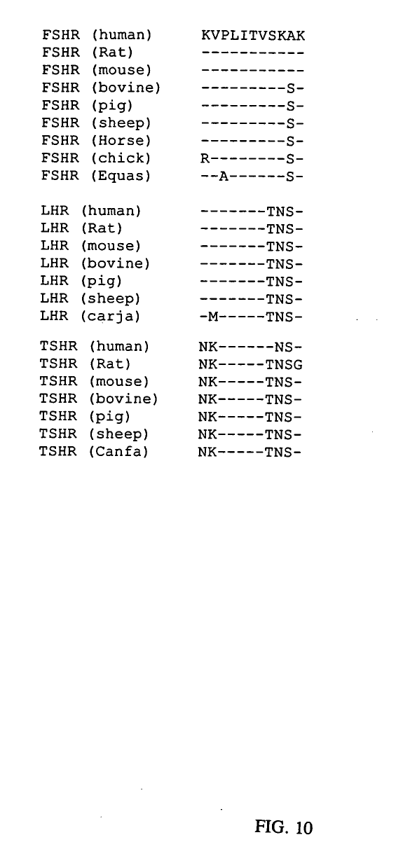

FIG. 11 shows the effects of Ala substitutions on IP production, Kd and cAMP induction. The exoloop 3 amino acids, K⁵⁸⁰ VPLITVSKAK⁵⁹⁰, were individually substituted with Ala, except the A588 G substitution, and the mutant receptors were assayed for IP total (IPₜ), IP₁, IP₂ and IP₃ (A). The ratios of Kd^{wt/mut} (blank bar), maximum cAMP^{mut/wt} (gray bar) and IPt^{mut/wt} (black bar) of the mutants were presented in bars (B). The ratios above 1.0 indicate that the mutants' binding affinity is better than the wild type affinity, and mutants' maximum cAMP and IP levels are higher than that of the wild type.

FIG. 12 shows a computer modeling of FSHR exoloop 3. FSHR exoloop 3 was modeled. (A) Stick model. (B) All of the exoloop 3 residues except V⁵⁸¹ and P⁵⁸², which are crucial for activation of PLCβ and production of IP, are presented in gold. (C) L⁵⁸³, I⁵⁸⁴ and K⁵⁹⁰ that are crucial for activation of AC and cAMP induction are presented in blue. (D) L⁵⁸³ and I⁵⁸⁴ that are crucial for hormone binding are presented in red.

FIG. 13 shows multiple substitutions of L⁵⁸³. L⁵⁸³ was substituted with a panel of amino acids with various side chains. The mutant receptors were expressed in HEK 293 cells and assayed for ¹²⁵I-FSH binding and FSH dependent cAMP induction. For hormone binding, counts of empty tubes (background) were ˜50 CPM and nonspecific binding was −75 CPM including background. Maximum specific binding CPM are normally in the range of 1,400-500 CPM. Nontransfected cells did not show specific binding of FSH. Each experiment was performed in duplicate and values were determined for Kd, receptors/cell, EC50 for cAMP synthesis, and maximum cAMP level. After experiments were repeated 6-10 times, the means and standard deviations were calculated. “NS” indicates “not significant”.

FIG. 14 shows multiple substitutions of I⁵⁸⁴, I⁵⁸⁴ was substituted with a panel of amino acids with various side chains, and the mutant receptors were expressed and assayed.

FIG. 15 shows multiple substitutions of P⁵⁸². P⁵⁸² was substituted with a panel of amino acids with various side chains, and the mutant receptors were expressed and assayed.

FIG. 16 shows multiple substitutions of K⁵⁹⁰. K⁵⁹⁰ was substituted with a panel of amino acids with various side chains, and the mutant receptors were expressed and assayed.

FIG. 17 shows an autoradiograph of photoaffinity labeled FSH. The peptide corresponding to the FSHR exoloop3 sequence (FSHR^{exo3}) was synthesized, derivatized with NHS-ABG and radio-iodinated to produce (¹²⁵I-AB-FSHR^{exo3}). (A) FSH was incubated with ¹²⁵I-AB-FSHR^{exo3} and irradiated with UV for increasing periods of time. The samples were solubilized in SDS under the reducing conditions and electrophoresed as described in Experimental Procedures. After electrophoresis, the gel was dried and autoradiographed using phosphoimager. The intensity of each band in a gel lane was measured, and the percentage of the labeled FSH band in a gel lane was calculated based on the total intensity of a gel lane, and presented in the bar graph. (B) Increasing concentrations of FSH were incubated with a constant amount of ¹²⁵I-AB-FSHR^{ex03} and irradiated with UV for 90 seconds. (C) Increasing concentrations of ¹²⁵I-AB-FSHR^{exo3} were incubated with a constant amount of FSH and irradiated with UV for 90 seconds.

FIG. 18 shows competitive inhibition of photoaffinity labeling. FSH was photoaffinity labeled with ¹²⁵I-AB-FSHR^{exo3} in the presence of increasing concentrations of unlabeled competitor peptides, exoloop 1 peptide (A), exoloop 2 peptide (B), exoloop 3 peptide (C), and FSHR⁹⁻⁴⁰ (D).

FIG. 19 shows identification of the labeled FSH subunit and futile labeling of denatured FSH. (A) Denatured FSH that is not capable of binding and activating FSHR was labeled with increasing concentrations of ¹²⁵I-AB-FSHR^{exo3}. (B) ¹²⁵I-AB-FSHR^{exo3} was to photoaffinity label FSH (lane 1), phospholipase A (lane 2), phospholipase C (lane 3), phospholipase D (lane 4), urokinase (lane 5) and human growth hormone (lane 6). (C) Inhibition of ¹²⁵I-FSH binding to the receptor on intact cells in the presence of unlabeled FSH (black square) and exoloop 3 peptide (open square). (D) FSH was photoaffinity labeled with ¹²⁵I-AB-FSHR^{exo3}, treated with PNGase F to deglycosylate it, and electrophoresed. The FSH a and b subunits separated in the lower band and upper band, respectively.

FIG. 20 shows hydrophobicity analysis of substitutions of P⁵⁸², L⁵⁸³, I⁵⁸⁴ and K⁵⁹⁰. (A) The Kd^{wt/mut} ratios of mutant receptors with a panel of amino acids at P⁵⁸², L⁵⁸³, I⁵⁸⁴ and K⁵⁹⁰ were compared in a bar graph. (B) The maximum cAMP mut/wt ratios of mutant receptors were compared in a bar graph.

FIG. 21 shows the effects of Ala substitutions on IP production. The exoloop 3 amino acids consisting of K⁵⁷³ VPLITVTNSK⁵⁸³ were individually substituted with Ala, and the mutant receptors were assayed for IPₜₒₜₐₗ (IPₜ), IP₁, IP₂ and IP₃ (A). “NS” stands for “not significant”.

FIG. 22 shows effects of Ala substitutions on hormone binding and cAMP induction. The Ala substitution mutants of the exoloop 3 amino acids were assayed for hormone binding and cAMP induction. “NS” stands for “not significant”.

FIG. 23 shows differential effects of Ala substitutions on hormone binding, cAMP and IP induction. To easily compare the Ala substitution effects on hormone binding, cAMP and IP induction, the ratios of Kd^{wild type}/Kd^{mutant} (Kd^{wt/mut}), maximum cAMP level of mutant/wild type (cAMP^{mut/wt}) and maximum IP^{t} level of mutant (IP^{mut/wt}) were displayed. Values over 1 reflect that mutants are better than the wild type.

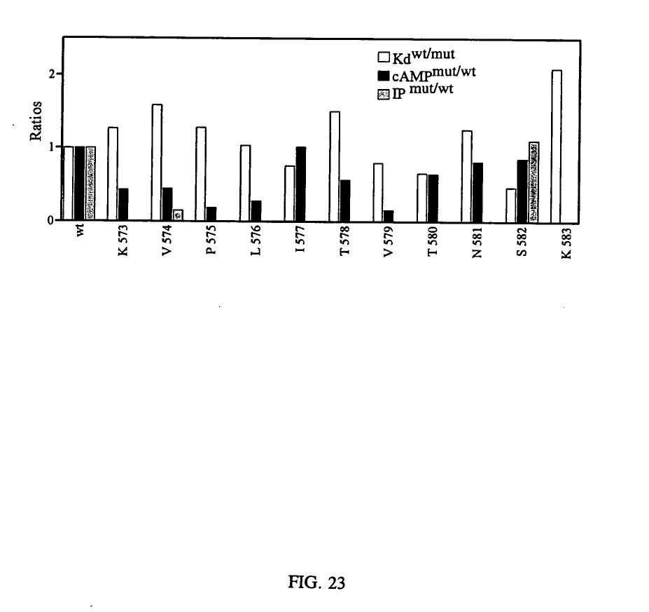

FIG. 24 shows comparison of LHR and FSHR. Exoloop 3 Ala substitution mutants of LHR and FSHR were compared for their Kd^{wt/mut}, cAMP^{mut/wt} and Ip^{mut/wt}. The amino acid positions of LHR are used. T⁵⁸⁰/S, N⁵⁸¹/K and S⁵⁸²/A are the diverse residues of LHR/FSHR. Ala of FSHR was substituted with Gly.

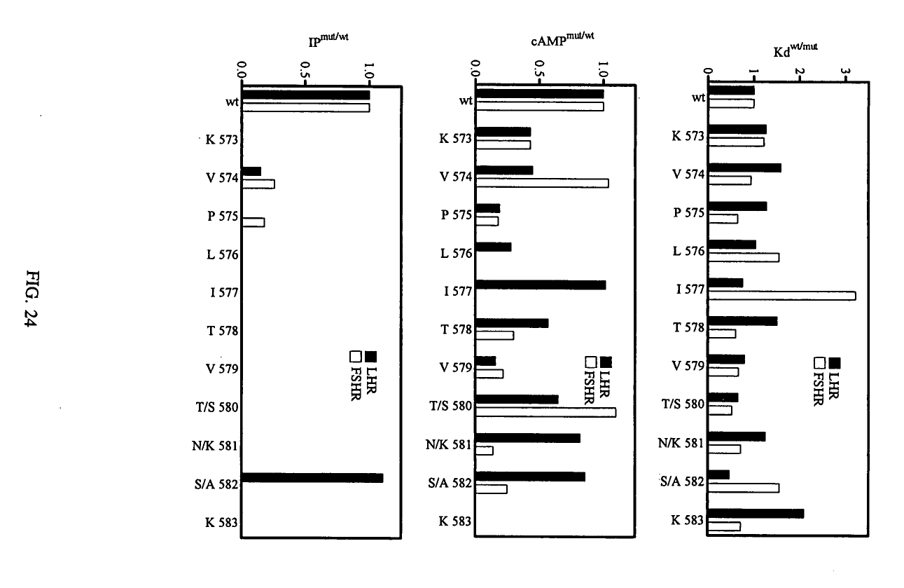

FIG. 25 shows multiple substitutions of K⁵⁸³ of LHR and FSHR. K⁵⁸³ of LHR and K⁵⁹⁰ of FSHR, the last residues of exoloop 3, were substituted with a panel of amino acids, and the mutant receptors were expressed and assayed as hormone binding, cAMP, and IP₁, IP₂, IP₃ and IPₜ. None of the mutants induced cAMP and IP species, although all of them were capable of binding their cognate hormones. Their Kd^{wt/mut} ratios are presented. “del” stands for deletion of the residues.

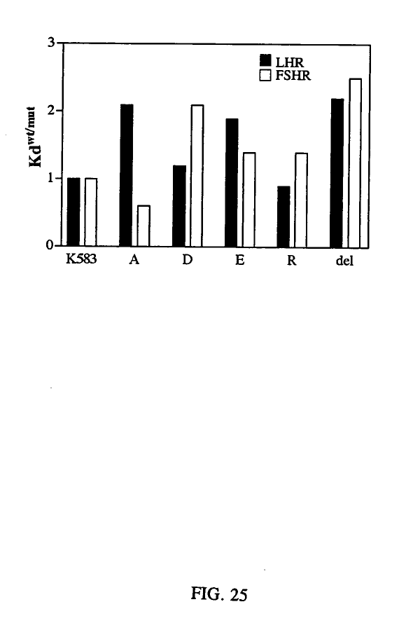

FIG. 26 shows an autoradiograph of photoaffinity labeled hCG subunits. The peptide corresponding to the LHR exoloop 3 sequence (LHR^{exo3}) was synthesized, derivatized with NHS-ABG and radioiodinated to produce (AB-¹²⁵ LHR^{exo3}). (A) Increasing concentration of hCG were incubated with AB-¹²⁵ I-LHR^{exo3} and irradiated with UV samples were solubilized in SDS under the reducing condition and electrophoresed. After electrophoresis, the gel was dried and autoradiographed using phosphoimager. The intensity of each band in a gel lane was measured. The percentage of the labeled hCG a and b subunit bands in a gel lane were calculated based the total intensity of a gel lane, and presented in the bar graph. (B) Increasing concentrations of ABG-¹²⁵LHR^{exo3} were incubated with a constant amount of hCG and irradiated with UV for 90 seconds. (C) A constant concentration of AB-¹²⁵I-LHR^{exo3} were incubated with a constant amount of hCG and irradiated with UV increasing time periods.

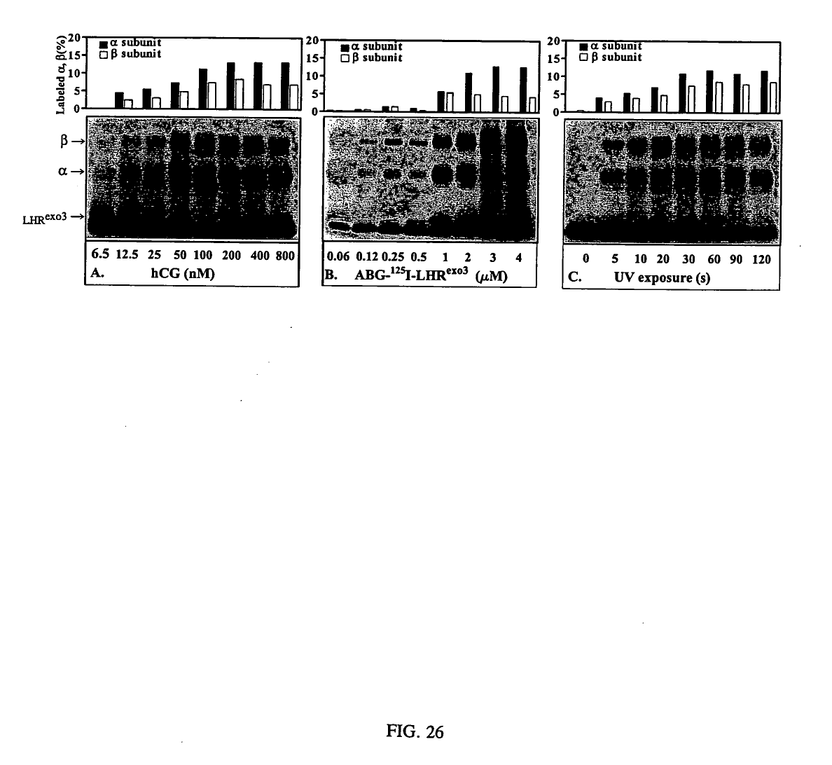

FIG. 27 shows photoaffinity labeling of denatured hCG. hCG was denatured by boiling in 8M urea for 30 min. Increasing concentrations of denatured hCG were incubated with AB-¹²⁵I-LHR^{exo3} and irradiated with UV for 90 (B) Increasing concentrations of ABG-¹²⁵I-LHR^{exo3} were incubated with a constant amount of denatured hCG irradiated with UV for 90 seconds. (C) Cells stably expressing LHR were incubated with ¹²⁵I-hCG in the presence increasing concentrations of nonlabeled hCG or nonlabeled LHR^{exo3}. The cells were washed and bound radioactivity was measured.

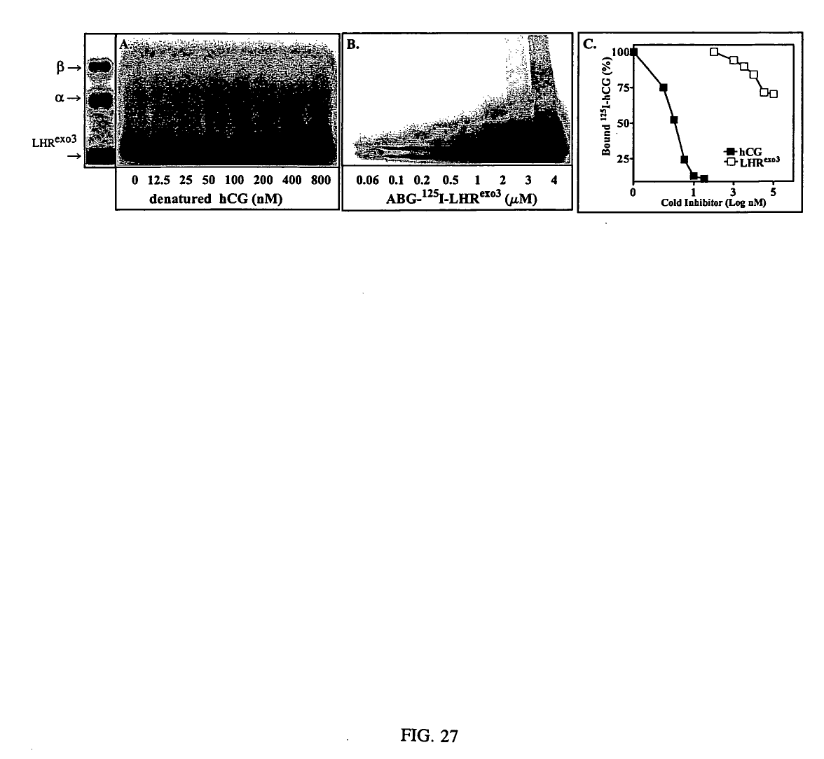

FIG. 28 shows photoaffinity labeling of LH. Increasing concentrations of human LH and denatured human LH were incubated with a constant amount of ABG-¹²⁵I-LHR^{exo3} and irradiated with UV for 1 minute. The samples were processed.

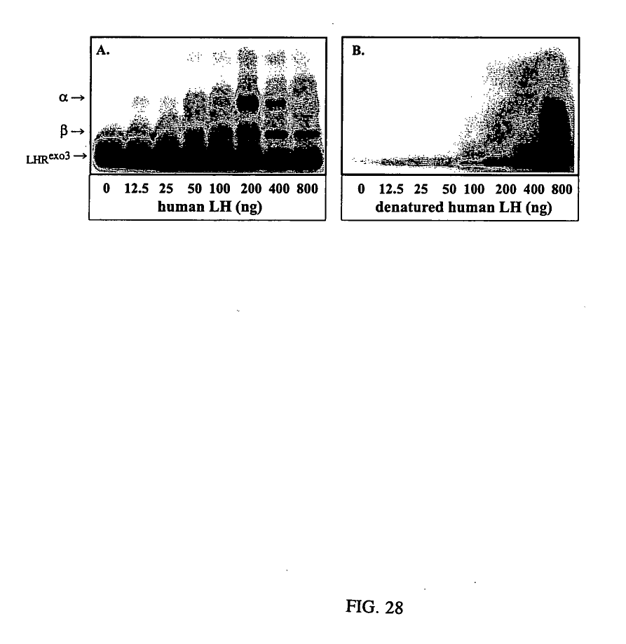

FIG. 29 shows specificity of photoaffinity labeling. hCG was photoaffinity labeled with AB-¹²⁵I-LHR^{exo3} in the presence of increasing concentrations of nonlabeled LHR^{Exo3} (A) and scrambled LHR^{Exo3} (B). (C) Various proteins (100 nM), hCG, phospholipase A (PLA), phospholipase C (PLC), phospholipase D (PLD) and urokinase (Uro) were incubated with ABG-¹²⁵I-LHR^{exo3} and irradiated with UV for 1 minute.

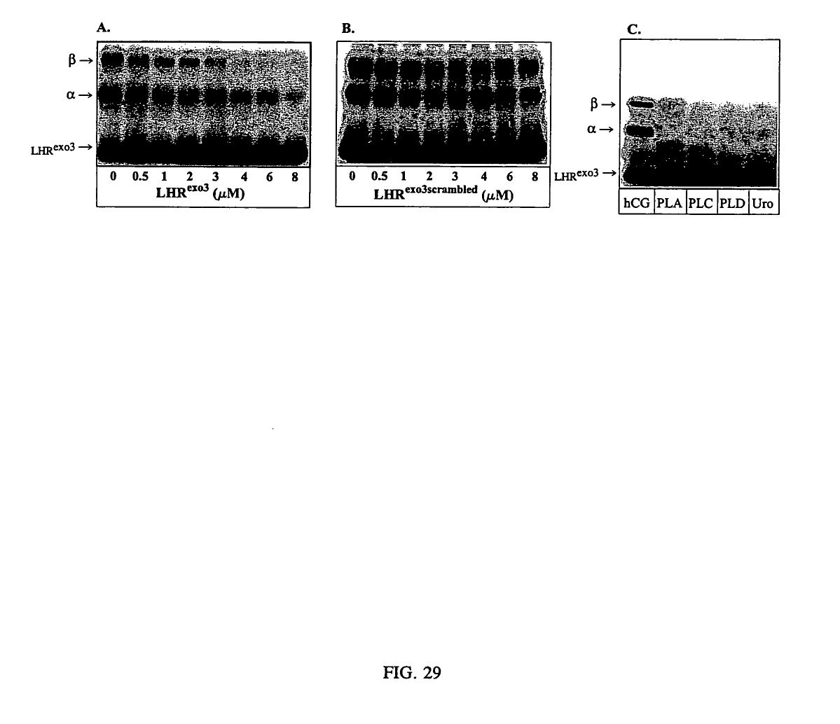

FIG. 30 shows photoaffinity labeling of FSH. (A) Human FSH were photoaffinity labeled with increasing concentrations of ABG-¹²⁵I-FSH, treated with PNGase F. ¹²⁵I-FSH was electrophoresed with and without digestion with PNGase F. (B) A constant amount denatured FSH was photoaffinity labeled with increasing concentrations of ABG-¹²⁵I-FSH^{exo3}. (C) Increasing concentrations of denatured FSH were photoaffinity label with a constant amount of ABG-¹²⁵I-FSH^{exo3}.

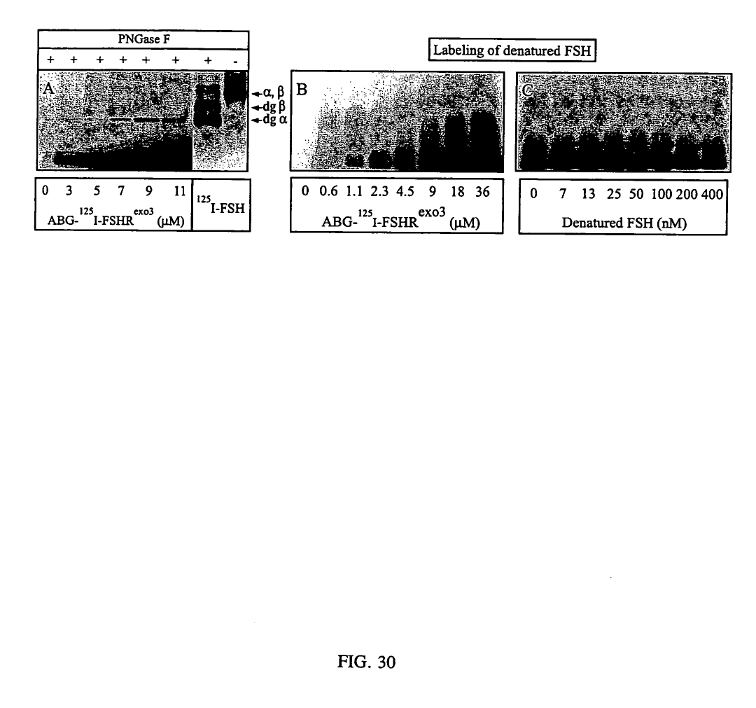

FIG. 31 shows the effects of other LHR peptides on photoaffinity labeling of hCG. hCG was photoaffinity labeled with AB-¹²⁵I-LHR^{exo3} in the presence of 4 mM of nonlabeled peptides, exoloop 1 peptide (Exo1), exoloop 2 peptide (Exo2), exoloop 3 peptide ((Exo3), scrambled exoloop 3 peptide (Exo3S), LHR¹⁷⁻³⁶ (17-36), LHR⁹⁶⁻¹¹⁵ (96-115) and LHR²⁴⁶⁻²⁶⁹ (246-269).

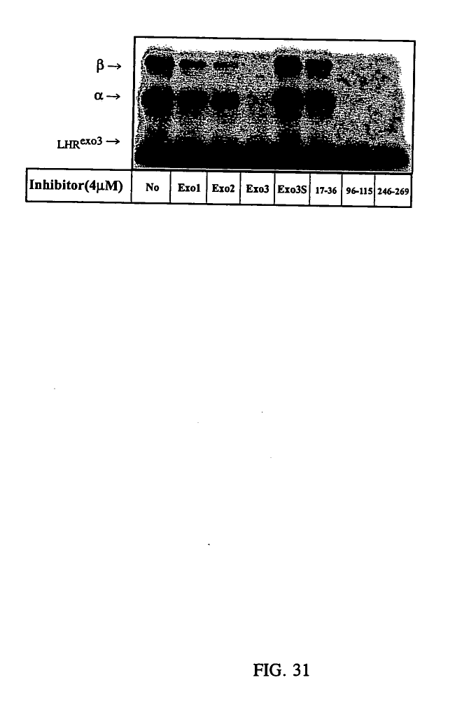

### DETAILED DESCRIPTION OF THE INVENTION

A. Definitions

In general, the terms in the present application are used consistently with the manner in which those terms are understood in the art.

By “gonadotropin” is meant hormones secreted by the anterior lobe of the pituitary gland that stimulate the normal functioning of the gonads and the secretion of sex hormones in both male and female animals. Gonadotropins include follicle stimulating hormone (FSH), leutinizing hormone (LH) and chorionic gonadotropin (CG).

By “leutinizing hormone receptor (LHR)” is meant the hormone receptor which binds both LH and CG. The LHR is also known as the leutinizing hormone/chorionic gonadotropin receptor (LHCGR or LH/CGR).

By “nucleic acid” is meant RNA, DNA, cDNA, recombinant RNA or DNA (i.e., rRNA and rDNA) that encodes a peptide, or is complementary to a nucleic acid sequence encoding such peptides, or hybridizes to either the sense or antisense strands of the nucleic acid and remains stably bound to it under appropriate stringency conditions.

By “modulate” and “regulate” is meant methods, conditions, or agents which increase or decrease the wild-type activity of an enzyme, inhibitor, signal transducer, receptor, transcription activator, co-factor, and the like. Preferably, the activity relates to gonadotropin and their receptors, as well as activity mediated thereby. This change in activity can be an increase or decrease of mRNA translation, mRNA or DNA transcription, and/or mRNA or protein degradation, which in turn corresponds to an increase or decrease in biological activity.

By “modulated activity or mediated activity” is meant any activity, condition, disease or phenotype which is modulated by a biologically active form of a gondadotropin. Modulation may be effected by affecting the concentration of biologically active protein, i.e., by regulating expression or degradation, or by direct agonistic or antagonistic effect such as, for example, through inhibition, activation, binding, or release of substrate, modification either chemically or structurally, or by direct or indirect interaction which may involve additional factors.

By “effective amount” or “dose effective amount” or “therapeutically effective amount” is meant an amount of an agent which modulates a biological activity of the proteins of the invention.

By “gonadotropin related disorder, condition or disease” and “gonadotropin mediated activity” is meant any state that involves gonadotropin activity or is a condition associated with a gonadotropin activity. This state can be a disease or disorder, such as cancer or acne or a condition, such as infertility or amenorrhea. The diseases, disorders and conditions can result from an abnormality in the gonadotropin hormone mechanism. However, any condition or disease that is not caused directly by gonadotropin activity but can be affected in some fashion by gonadotropins is contemplated to be “gonadotropin-related”. The terms “disease” and “disorder” are used interchangeably for the purposes of this invention.

By “hormone/receptor” is meant FSH/FSHR and CG/LHR. Any time the phrase hormone and/or receptor is used, both FSH/FSHR and CG/LHR are indicated.

II. Introduction

A mechanism for the control of the interaction between gonadotropin hormones and their receptors has been discovered. The complete molecular mechanism of gonadotropin hormone binding is not fully understood. However, recent studies have identified specific sites in the gonadotropin receptors which bind to gonadotropins and result in hormone signal.

In one embodiment of the present invention, the activity of CG, LH and FSH may be modulated in a subject. This modulation may take place, for example, through binding with a peptide aptamer of the present invention. In addition, modulation of the receptors, LHR and FSHR, may be achieved by binding with a peptide aptamer of the present invention. In another embodiment of the present invention, the CG/LH/LHR and FSH/FSHR interaction may be modulated by a reagent of the present invention.

The methods of modulation of hormone/receptor interaction of the present invention comprise administering a therapeutically effective amount of an agent which modulates activity, wherein the agent can bind to the exoloop 1, exoloop 2 or exoloop 3 of the LHR or FSHR creating a agent/exoloop complex or the agent can bind to the hormone (i.e., CG, LH or FSH) at the hormone binding domain creating an agent/hormone complex. More specifically, the agent modulates hormone activity or hormone/receptor interaction by modulating interaction of the leucine rich motif of the hormone receptor with the appropriate hormone.

The subject can be a vertebrate or an invertebrate. The subject may be a canine, a feline, an ovine, a primate, an equine, a porcine, a caprine, a camelid, an avian, a bovine, amphibian, fish or a murine organism. Preferably, the subject is a primate. More preferably, the subject is human. A more preferred subject is a human, male or female. Thus, the CG is preferably hCG.

The agents contemplated by the present invention include, but are not limited to, FSH or a biologically active fragment thereof, LH or a biologically active fragment thereof, CG or a biologically active fragment thereof. The agent can act by blocking CG and FSH from binding to LHR and FSHR by either binding to the hormone at the binding domain, or binding to the receptor at the exoloop 1, exoloop 2 or exoloop 3. The agent may also be an antisense molecule which binds to a nucleic acid encoding CG or FSH or a fragment of the nucleic acid encoding CG or FSH.

Alternative embodiments provide methods for screening potential drugs and other therapies directed to the treatment of gonadotropin related diseases and conditions in both males and females, as well as drugs and therapies useful as contraceptives. The invention also includes drugs and therapies discovered and developed by the use of the reagents and/or methods of the present invention.

Gonadotropin related diseases, disorders and conditions which may be treated by the methods and compositions of the present invention occur in both males and females of any mammal, preferably in humans. The disorders occurring in men include, but are not limited to, male pseudohermaphroditism, microphallus, gynecomastia, bilateral anorchia, absence of Leydig's cells, cryptorchidism, Noonan's syndrome and myotonic dystrophy, delayed puberty, prostate cancer, precocious puberty, acne and impotence.

The disorders occurring in women include, but are not limited to, primary and secondary amenorrhea, delayed puberty, precocious puberty, endometriosis, acne, uterine myoma, ovarian and mammary cystic diseases, and breast and gynecological cancers.

The present invention also contemplates a method of contraception. This method of contraception comprises administering to a subject an amount of an agent effective at preventing conception. The agent modulates either CG, LH or FSH activity or CG or FSH activity with the LHR or FSHR. The subject may be either male or female.

The present invention also includes methods of screening for compounds which modulate the interaction between CG, LH and LHR or FSH and FSHR. These methods comprise (a) attaching the hormone or a biologically active polypeptide fragment thereof to a substrate; (b) exposing the hormone or the biologically active polypeptide fragment thereof to an agent; and (c) determining whether said agent bound to the hormone or the biologically active polypeptide fragment thereof and further determining whether said agent modulates the interaction between the hormone and its receptor. The present invention also contemplates the compounds identified by these methods.

The present invention also includes compositions for treating gonadotropin hormone related diseases comprising a pharmaceutically effective amount of a hormone receptor modulating compound and a pharmaceutically acceptable excipient.

The compound which modulates the hormone receptor of interest can be the hormone or a biologically active fragment thereof. The composition may be any compound identified through the methods of this invention. The composition can be any agent which binds to the hormone preventing its interaction with the receptor at the exoloop 1, exoloop 2 or exoloop 3. The agent may be a monoclonal antibody or immunologically active fragment thereof which binds to the hormone thereby modulating the activity of the receptor and a pharmaceutically acceptable excipient.

In addition, the present invention includes a method of modulating at least one activity of CG, LH or FSH comprising administering an effective amount of an agent which modulates at least one activity of CG, LH or FSH. The modulated activity is selected from the group consisting of stimulation of progesterone, androgen and estrogen and stimulation of development of the male and female gonads, follicles, placenta, maturation of oocytes and sperm and growth of cells and tissue.

The present invention also includes a method of detecting the ability of a test sample to affect the binding interaction of a first peptide and a second peptide of a peptide binding pair that bind through extracellular interaction in their natural environment, comprising: culturing at least one yeast cell, wherein the yeast cell comprises: a nucleotide sequence encoding a first heterologous fusion protein comprising the first peptide or a segment thereof joined to a transcriptional activation protein DNA binding domain; a nucleotide sequence encoding a second heterologous fusion protein comprising the second peptide or a segment thereof joined to a transcriptional activation protein transcriptional activation domain; wherein binding of the first peptide or segment thereof and the second peptide or segment thereof reconstitutes a transcriptional activation protein; and a reporter gene activated under positive transcriptional control of the reconstituted transcriptional activation protein, wherein expression of the reporter gene produces a selected phenotype; incubating a test sample with the yeast cell under conditions suitable to detect the selected phenotype; and detecting the ability of the test sample to affect the binding interaction of the peptide binding pair by determining whether the test sample affects the expression of the reporter gene which produces the selected phenotype, and wherein said first peptide is a CG or FSH peptide and said second peptide is a LHR or FSHR peptide. The yeast is preferably Saccharomyces. The Saccharomyces I is preferably Saccharomyces cerevisiae.

The yeast cell may further comprise at least one endogenous nucleotide sequence selected from the group consisting of a nucleotide sequence encoding the transcriptional activation protein DNA binding domain, a nucleotide sequence encoding the transcriptional activation protein transcriptional activation domain, and a nucleotide sequence encoding the reporter gene, wherein at least one of the endogenous nucleotide sequences is inactivated by mutation or deletion. The peptide binding pair may comprise a ligand and a receptor to which the ligand binds.

The transcriptional activation protein may be Gal4, Gcn4, Hap1, Adr1, Swi5, Ste12, Mcm1, Yap1, Ace1, Ppr1, Arg81, Lac9, Qa1F, VP16, or a mammalian nuclear receptor. At least one of the heterologous fusion proteins is expressed from an autonomously-replicating plasmid.

The reporter gene may be selected from the group consisting of lacZ, a gene encoding luciferase, a gene encoding green fluorescent protein (GFP), and a gene encoding chloramphenicol acetyltransferase. The peptide binding pair is other than an antigen and a corresponding antibody.

III. Polypeptides

The polypeptides contemplated for use in this invention include those which modulate gonadotropin activity.

A set of peptide aptamers can be identified from a library of random peptides constrained and presented in a thioredoxin A (trxa) scaffold. The present invention contemplates a nucleic acid encoding a CG aptamer comprising a nucleic acid encoding a scaffold protein in-frame with the activation domain of Gal4 that is in-frame with a nucleic acid which encodes for a CG amino acid sequence. Nucleic acid sequences encoding the same are also contemplated by the present invention.

Peptide aptamers are powerful new tools for molecular medicine as reviewed by Hoppe-Seyler& Butz, 2000, (J. Mol. Med., 78:426-430); Brody and Gold, 2000 (Rev. Mol. Biotech., 74:5-13); and Colas, 2000 (Curr. Opin. in Chem. Biol. 4:54-9) and references cited therein. Briefly, peptide aptamers have been shown to be highly specific reagents capable of binding in vivo. As such, peptide aptamers provide a method of modulating the function of a protein and may serve as a substitute for conventional knock-out methods or complete loss of function. Peptide aptamers are also useful reagents for the validation of targets for drug development and may be used as therapeutic compounds directly or provide the necessary foundation for drug design. Once identified, the peptide insert may be synthesized and used directly or incorporated into another carrier molecule. References reviewed and cited by Brody and Gold (2000, supra) describe demonstrated therapeutic and diagnostic applications of peptide aptamers.

The peptide aptamers of the present invention are useful reagents in binding of CG, LH and FSH and thereby modulation of the interaction between CG and LHR and FSH and FSHR. The peptide aptamers refers to the peptide constrained by the thioredoxin scaffold. The aptamers are also contemplated as therapeutic agents to treat gonadotropin related diseases and conditions.

Additional CG and FSH interacting proteins can be identified in a yeast-two-hybrid screen using the FSH and CG bait. Additional methods would be know, see, e.g., Yeast Hybrid Technologies, 2000 (Zhu et al., eds., Eaton Publishing, Natick, Mass.) and Two-Hybrid Systems: Methods and Protocols, 2001 (MacDonald ed., Humana Press, Totowa, N.J.).

Novel peptides can be designed based on the existing sequences and synthesized using natural and unnatural amino acids to improve stability and efficiency. They may have a single peptide-chain or more than one peptide chain attached to a chemical matrix. These multi-peptide chains may simulate the partial or whole structure of the exoloops or the cytoplasmic loops (cytoloops) attached to the transmembrane helices of the receptors. Peptides may be modified to enhance specific conformations and increase the specificity and binding affinity and stability. The N-terminus and C-terminus of exoloops 1, 2 and 3 may be covalently crosslinked to stabilize the loop structure.

IV. Nucleic Acid Molecules

The present invention further provides nucleic acid molecules that encode polypeptides and proteins which interact with CG, LH and LHR and FSH and FSHR to modulate the activities of these hormones. Preferred embodiments provide nucleic acids encoding for the identified fragment of CG protein and FSH protein, polypeptide aptamers of CG, LH, FSH, FSHR and LHR and related fusion proteins, preferably in isolated or purified form. The nucleic acid may encode a polypeptide sharing at least 75% sequence identity, preferably at least 80%, and more preferably at least 85%, with the peptide sequences; 90%, 95%, 96%, 97%, 98%, and 99% identity or greater are also contemplated. Specifically contemplated are genomic DNA, cDNA, mRNA, antisense molecules, enzymatically active nucleic acids (e.g., ribozymes), as well as nucleic acids based on an alternative backbone or including alternative bases, whether derived from natural sources or synthesized. Such hybridizing or complementary nucleic acids, however, are defined further as being novel and nonobvious over any prior art nucleic acid including that which encodes, hybridizes under appropriate stringency conditions, or is complementary to a nucleic acid encoding a protein according to the present invention.

As used herein, the terms “hybridization” and “specificity” in the context of nucleotide sequences are used interchangeably. The ability of two nucleotide sequences to hybridize to each other is based upon the degree of complementarity of the two nucleotide sequences, which in turn is based on the fraction of matched complementary nucleotide pairs. The more nucleotides in a given sequence that are complementary to another sequence, the greater the degree of hybridization of one to the other. The degree of hybridization also depends on the conditions of stringency which include temperature, solvent ratios, salt concentrations, and the like. In particular, “selective hybridization” pertains to conditions in which the degree of hybridization of a polynucleotide of the invention to its target would require complete or nearly complete complementarity. The complementarity must be sufficiently high so as to assure that the polynucleotide of the invention will bind specifically to the target nucleotide sequence relative to the binding of other nucleic acids present in the hybridization medium. With selective hybridization, complementarity will be 90-100%, preferably 95-100%, more preferably 100%.

“Stringent conditions” are those that (1) employ low ionic strength and high temperature for washing, for example: 0.015 M NaCl, 0.0015 M sodium titrate, 0.1% SDS at 50° C.; or (2) employ during hybridization a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal.

As used herein, a nucleic acid molecule is said to be “isolated” OR “purified” when the nucleic acid molecule is substantially separated from contaminant nucleic acids encoding other polypeptides from the source of nucleic acid. This can include genomic nucleic acid which occur immediately upstream or downstream from the nucleic acid of interest.

The present invention further provides fragments of the encoding nucleic acid molecule. As used herein, a fragment of an encoding nucleic acid molecule refers to a small portion of the entire protein encoding sequence. The size of the fragment will be determined by the intended use. For example, if the fragment is chosen so as to encode a biologically active portion of the protein, the fragment will need to be large enough to encode the functional region(s) of the protein. If the fragment is to be used as a nucleic acid probe or PCR primer, then the fragment length is chosen so as to obtain a relatively small number of false positives during probing/priming.

Fragments of the encoding nucleic acid molecules of the present invention (i.e., synthetic oligonucleotides) that are used as probes or specific primers for the polymerase chain reaction (PCR), or to synthesize gene sequences encoding proteins of the invention can easily be synthesized by chemical techniques, for example, the phosphotriester method of Matteucci et al., 1981 (J. Am. Chem. Soc. 103: 3185-3191) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The polypeptide encoding nucleic acid molecules of the present invention may further be modified to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can employ any of the art known labels to obtain a labeled encoding nucleic acid molecule.

Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

Antisense molecules corresponding to the polypeptide coding or complementary sequence may be prepared. Methods of making antisense molecules which bind to mRNA, form triple helices or are enzymatically active and cleave TSG RNA and single stranded DNA (ssDNA) are known in the art. See, e.g., Antisense and Ribozyme Methodology: Laboratory Companion (Ian Gibson, ed., Chapman & Hall 1997) and Ribozyme Protocols: Methods in Molecular Biology (Phillip C. Turner, ed., Humana Press, Clifton, N.J. 1997).

V. rDNA Molecules for Polypeptides

The present invention further provides recombinant DNA molecules (rDNAs) that contain a polypeptide coding sequence. As used herein, a rDNA molecule is a DNA molecule that has been subjected to molecular manipulation in situ. Methods for generating rDNA molecules are well known in the art, for example, see Sambrook et al., 1989, MOLECULAR CLONING, A LABORATORY MANUAL, Cold Spring Harbor Laboratory, Cold Spring Harbor, N.Y. and Current Protocols in Molecular Biology, 2000, Ausebel et al., eds. John Wiley & Sons, NY. In the preferred rDNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium. Preferred promoters include yeast promoters, which include promoter regions for metallothionein, 3-phosphoglycerate kinase or other glycolytic enzymes (e.g., enolase or glyceraldehyde-3-phosphate dehydrogenase), promoters for enzymes responsible for maltose and galactose utilization, and others. Vectors and promoters suitable for use in yeast expression are further described in EP 73,675A. Appropriate non-native mammalian promoters might include the early and late promoters from SV40 (Fiers et al., 1978 (Nature, 273:113)) or promoters derived from murine Moloney murine leukemia virus, mouse tumor virus, avian sarcoma viruses, adenovirus II, bovine papilloma virus or polyoma viruses. In addition, the construct may be joined to an amplifiable gene (e.g., DHFR) so that multiple copies of the gene may be made. For appropriate enhancer and other expression control sequences, see also Enhancers and Eukaryotic Gene Expression, 1983, Cold Spring Harbor Press, Cold Spring Harbor, N.Y.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors with a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 available from Biorad Laboratories, (Richmond, Calif.), and pPL and pKK223 available from Pharmacia (Piscataway, N.J.).

Expression vectors compatible with eukaryotic cells, preferably those compatible with vertebrate cells, can also be used to form a rDNA molecule that contains a coding sequence. Eukaryotic cell expression vectors are well known in the art and are available from several commercial sources. Typically, such vectors are provided containing convenient restriction sites for insertion of a desired DNA segment. Typical of such vectors are pSVL and pKSV-10 (Pharmacia), pBPV-1/pML2d (International Biotechnologies, Inc.), vector systems that include Histidine Tags and periplasmic secretion, or other vectors described in the art. Preferred vectors for expressing sequences that modulate gonadotropin hormones and/or their receptors include pcDNA, pcDNA4/HisMx and their derivatives (Invitrogen).

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene (Southern et al., 1982 (J. Mol. Anal. Genet. 1: 327-341)). Alternatively, the selectable marker can be present on a separate plasmid, and the two vectors introduced by co-transfection of the host cell, and selected by culturing in the appropriate drug for the selectable marker.

VI. Host Cells Containing an Exogenously Supplied rDNA Nucleic Acid Molecule

The present invention further provides host cells transformed with a nucleic acid molecule that encodes a polypeptide or protein of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferably, the vertebrates are mammals. Preferred eukaryotic host cells include but are not limited to, human embryonic kidney cells 297 (HEK 297), Chinese hamster ovary (CHO) cells (ATCC No. CCL61), NIH Swiss mouse embryo cells NIH/3T3 (ATCC No. CRL 1658), baby hamster kidney cells (BHK), and other like eukaryotic tissue culture cell lines.

Any prokaryotic host can be used to express a rDNA molecule encoding a protein of the invention. The preferred prokaryotic host is E. coli.

Transformation of appropriate cell hosts with a recombinant DNA (rDNA) molecule of the present invention is accomplished by well known methods that typically depend on the type of vector used and host system employed. With regard to transformation of prokaryotic host cells, electroporation and salt treatment methods are typically employed; see, for example, Cohen et al., 1972 (Proc. Natl. Acad. Sci. USA 69: 2110); Maniatis et al., 1982; and Sambrook et al., 1989. With regard to transformation of vertebrate cells with vectors containing rDNAs, electroporation, cationic lipid or salt treatment methods are typically employed; see, for example, Graham et al., 1973 Virol. 52: 456; Wigler et al., 1979 Proc. Natl. Acad. Sci. USA 76: 1373-76.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, 1975 J. Mol. Biol. 98: 503, or Berent et al., 1985 Biotech. 3: 208. Alternatively, the cells can be cultured to produce the proteins encoded by the rDNA and the proteins harvested and assayed, using for example, any suitable immunological method. See, e.g., Harlow et al., 1988 and Hadow et al., Using Antibodies: A Laboratory Manual, 1998 (CSH Labs) and Ausubal, Short Protocols in Molecular Biology, 1999 (John Wiley & Sons).

Recombinant DNA can also be utilized to analyze the function of coding and non-coding sequences. Sequences that modulate the translation of the mRNA can be utilized in an affinity matrix system to purify proteins obtained from cell lysates that associate with the CG expression control sequence. Synthetic oligonucleotides would be coupled to the beads and probed with the lysates, as is commonly known in the art. Associated proteins could then be separated using, for example, a two dimensional SDS-PAGE system. Proteins thus isolated could be further identified using mass spectroscopy or protein sequencing. Additional methods would be apparent to the skilled artisan.

VII. Production of Recombinant Peptides and Proteins using a cDNA or Other Recombinant Nucleic Acids

The invention also relates to nucleic acid molecules which encode a CG protein and polypeptide fragments thereof, and proteins and polypeptides which bind to CG and FSH (e.g., LHR and FSHR) and analog molecules. Further, the invention relates to nucleic acid molecules which encode a LHR and FSHR protein and polypeptide fragments thereof, and proteins and polypeptides which bind to LHR and FSHR (e.g., CG and FSH) and analog molecules. The polypeptides of the present invention include the exoloop 1, exoloop 2 or exoloop 3 and polypeptide fragments thereof, CG binding proteins and polypeptides thereof. Preferably these proteins are mammalian proteins, and most preferably human proteins and biologically active fragments thereof. Alternative embodiments include nucleic acid molecules encoding polypeptide fragments having a consecutive amino acid sequence of at least 3, 5, 10, 15, 20, 25, 30 or 40 amino acid residues from a common polypeptide sequence; amino acid sequence variants of a common polypeptide sequence wherein an amino acid residue has been inserted N- or C-terminal to, or within, the polypeptide sequence or its fragments; and amino acid sequence variants of the common polypeptide sequence or its fragments, which have been substituted by another conserved residue. Recombinant nucleic acid molecules which encode polypeptides include those containing predetermined mutations by, e.g., homologous recombination, site-directed or PCR mutagenesis, and recombinant CG or FSH proteins or polypeptide fragments of other animal species, including but not limited to vertebrates (e.g., rabbit, rat, murine, porcine, camelid, reptilian, caprine, avian, fish, bovine, ovine, equine and non-human primate species), and alleles or other naturally occurring variants and homologs of CG or FSH binding proteins of the foregoing species and of human sequences. Also contemplated herein are derivatives of the commonly known CG or FSH or its fragments, wherein CG or FSH or its fragments have been covalently modified by substitution, chemical, enzymatic, or other appropriate means with a moiety other than a naturally occurring amino acid (for example a detectable moiety such as an enzyme or radioisotope) and soluble forms of CG or FSH.

The nucleic acid molecules encoding CG and FSH binding proteins, the receptor binding domain fragment of CG or FSH, the leucine rich repeats of the exodomain of the LHR, FSHR or other polypeptides of the present invention are preferably those which share a common biological activity (e.g., the modulation of the interaction between CG and the LHR and FSH and FSHR). The polypeptides of the present invention include those encoded by a nucleic acid molecule with silent mutations, as well as those nucleic acids encoding a biologically active protein with conservative amino acid substitutions, allelic variants, and other variants of the disclosed polypeptides which maintain at least one CG activity, such as the stimulation of the gonadal development.

The amino acid compounds of the invention are polypeptides which are partially defined in terms of amino acid residues of designated classes. Polypeptide homologs would include conservative amino acid substitutions within the amino acid classes described below. Amino acid residues can be generally sub-classified into four major subclasses as follows:

Acidic: The residue has a negative charge due to loss of H⁺ ion at physiological pH, and the residue is attracted by aqueous solution so as to seek the surface positions in the conformation of a peptide in which it is contained when the peptide is in aqueous medium, at physiological pH.

Basic: The residue has a positive charge due to association with H⁺ ion at physiological pH, and the residue is attracted by aqueous solution so as to seek the surface positions in the conformation of a peptide in which it is contained when the peptide is in aqueous medium at physiological pH.

Neutral/non-polar: The residues are not charged at physiological pH, but the residue is attracted by aqueous solution so as to seek the outer positions in the conformation of a peptide in which it is contained when the peptide is in aqueous medium. These residues are also designated “hydrophobic.”

Neutral/polar: The residues are not charged at physiological pH, but the residue is attracted by aqueous solution so as to seek the outer positions in the conformation of a peptide in which it is contained when the peptide is in aqueous medium.

It is understood, of course, that in a statistical collection of individual residue molecules some molecules will be charged, and some not, and there will be an attraction for or repulsion from an aqueous medium to a greater or lesser extent. To fit the definition of “charged”, a significant percentage (at least approximately 25%) of the individual molecules are charged at physiological pH. The degree of attraction or repulsion required for classification as polar or nonpolar is arbitrary and, therefore, amino acids specifically contemplated by the invention have been classified as one or the other. Most amino acids not specifically named can be classified on the basis of known behavior.

Amino acid residues can be further subclassified as cyclic or noncyclic, and aromatic or non-aromatic, self-explanatory classifications with respect to the side chain substituent groups of the residues, and as small or large. The residue is considered small if it contains a total of four carbon atoms or less, inclusive of the carboxylcarbon. Small residues are, of course, always nonaromatic.

The gene-encoded secondary amino acid proline, although technically within the group neutral/nonpolar/large/cyclic and nonaromatic, is a special case due to its known effects on the secondary conformation of peptide chains, and is not, therefore, included in this defined group.

Other amino acid substitutions of those encoded in the gene can also be included in peptide compounds within the scope of the invention and can be classified within this general scheme according to their structure.

All of the compounds of the invention may be in the form of the pharmaceutically acceptable salts or esters. Salts may be, for example, Na⁺, K⁺, Ca⁺², Mg⁺² and the like; the esters are generally those of alcohols of 1-6 carbons.

The present invention further provides methods for producing a protein of the invention using nucleic acid molecules herein described. In general terms, the production of a recombinant form of a protein typically involves the following steps:

First, a nucleic acid molecule is obtained that encodes CG, or any CG sequence. Particularly for CG binding peptides, the nucleotides encoding the peptide are incorporated into a nucleic acid in the form of an in-frame fusion, insertion into or appended to a thioredoxin coding sequence. The coding sequence (ORF) is directly suitable for expression in any host, as it is not interrupted by introns.

These DNAs can be transfected into host cells such as eukaryotic cells or prokaryotic cells. Eukaryotic hosts include mammalian cells and vertebrate (e.g., osteoblasts, osteosarcoma cell lines, Drosophila S2 cells, hepatocytes, tumor cell lines and other bone cells of any mammal, as well as insect cells, such as Sf9 cells using recombinant baculovirus).

Alternatively, proteins and polypeptides of the present invention can be expressed in an heterologous system. The human cell line GM637, an SV-40 transformed human fibroblast, can be transfected, with a plasmid containing a CG ligand binding domain coding sequence under the control of the chicken actin promoter. See Reis et al., 1992 (EMBO J. 11: 185-193). Such transfected cells can be used as a source of CG ligand binding domain in functional assays. Alternatively, polypeptides encoding only a portion of CG can be expressed alone or in the form of a fusion protein. For example, CG derived peptides can be expressed in bacteria (e.g., E. coli) as GST- or His-Tag fusion proteins. These fusion proteins are then purified and can be used to generate polyclonal antibodies or can be used to identify CG ligands.

The nucleic acid coding sequence is preferably placed in operable linkage with suitable control sequences, as described above, to form an expression unit containing the protein encoding open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

VIII. Methods to Identify Agents that Modulate at Least One Activity of CG, LH, LHR, FSH or FSHR

Another embodiment of the present invention provides methods for identifying agents that modulate at least one activity of CG, LHR, FSH and FSHR proteins or preferably which specifically modulate an activity of a CG/LHR complex, FSH/FSHR complex or a biologically active fragment of CG or FSH (e.g., the fragment which comprises the domain which binds to the LRRs on the LHR or FSHR). Such methods or assays may utilize any means of monitoring or detecting the desired activity as would be known in the art (See, e.g., Wu et al., 2000 (Curr. Biol. 10:1611-4); Fedi et al., 1999 (J. Biol. Chem. 274:19465-72); Grotewold et al., 1999 (Mech. Dev. 89:151-3); Shibata et al., 2000 (Mech. Dev. 96: 243-6); Wang et al. (2000 Oncogene 19: 1843-8); and Glinka et al., 1998 (Nature 391: 357-62)).

In one embodiment, the relative amounts of CG of a cell population that has been exposed to the agent to be tested is compared to an un-exposed control cell population. Antibodies can be used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, as would be known in the art. See, e.g., Ed Harlow and David Lane, ANTIBODIES: A LABORATORY MANUAL 1988 (Cold Spring Harbor, N.Y.) and Ed Harlow and David Lane, 1998 USING ANTIBODIES: A LABORATORY MANUAL (Cold Spring Harbor, N.Y.).

Natural and synthetic chemicals, small and large, may be screened for their solubility, stability and effectiveness as modulators of gonadotropins, exodomains, exoloops, transmembrane helices and cytoloops. A synthetic or natural chemical can interact with exoloops and modulate them. A chemical compound may contain part of whole of exoloop 1, 2 or 3, or a combination thereof, and modulate the exodomain or endodomain.

1. Antibodies and Antibody Fragments

Polyclonal and monoclonal antibodies and immunologically active fragments of these antibodies which bind to certain domains of FSH, LH and CG can be prepared as would be known in the art. These domains include, but are not limited to, the loops 1-3 and the C-terminal tail of the α subunits, and loops 1-3, the seat belt and the C-terminal tail of the β subunits. See Nature, 369:455-461. For example, suitable host animals can be immunized using appropriate immunization protocols and the peptides, polypeptides or proteins of the invention. Peptides for use in immunization are typically about 8-40 residues long. If necessary or desired, the polypeptide immunogens can be conjugated to suitable carriers. Methods for preparing immunogenic conjugates with carriers such as bovine serum albumin (BSA), keyhole limpet hemocyanin (KLH), or other carrier proteins are well known in the art (See, Harlow et al., 1988 and 1998). In some circumstances, direct conjugation using, for example, carbodiimide reagents, may be effective; in other instances linking reagents such as those supplied by Pierce Chemical Co., Rockford, Ill., may be desirable to provide accessibility to the polypeptide or hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

Anti-peptide antibodies can be generated using synthetic peptides, for example, the peptides derived from the sequence of the domain of the CG and FSH which binds to the exoloop 1, exoloop 2 or exoloop 3 on the LHR or FSHR. Synthetic peptides can be as small as 2-3 amino acids in length, but are preferably at least 3, 5, 10, 15, 20, 25, 30, 35, 40, 45, 50 or more amino acid residues long. Such peptides can be determined using programs such as DNAStar. Polyclonal anti-CG peptide antibodies can then be purified, for example using Actigel beads containing the covalently bound peptide.

While the polyclonal antisera produced in this way may be satisfactory for some applications, for pharmaceutical compositions, use of monoclonal preparations is preferred. Immortalized cell lines which secrete the desired monoclonal antibodies may be prepared using the standard method of Kohler and Milstein or modifications which effect immortalization of lymphocytes or spleen cells, as is generally known (See, e.g., Harlow et al., 1988 and 1998). The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonal antibodies, which contain the immunologically significant portion, can be used as agonists or antagonists of CG activity. Use of immunologically reactive fragments, such as the Fab, scFV, Fab′, of F(ab′)₂ fragments are often preferable, especially in a therapeutic context, as these fragments are generally less immunogenic than the whole immunoglobulin.

The antibodies or fragments may also be produced, using current technology, by recombinant means. Regions that bind specifically to the desired regions of CG or FSH (such as the regions the bind to exoloops 1-3) can also be produced in the context of chimeras with multiple species origin. Antibody reagents so created are contemplated for use diagnostically or as stimulants or inhibitors of CG activity.

In one embodiment, antibodies against CG bind CG with high affinity, i.e., ranging from 10⁻⁵ to 10⁻⁹ M. Preferably, the anti-CG antibody will comprise a chimeric, primate, primatized®, human or humanized antibody. Also, the invention embraces the use of antibody fragments, e.g., Fab, Fv, Fab′, scFv, F(ab′)₂, and aggregates thereof.

A primatized® antibody refers to an antibody with primate variable regions, e.g., CbR's, and human constant regions. Preferably, such primate variable regions are derived from an Old World monkey.

A humanized antibody refers to an antibody with substantially human framework and constant regions, and non-human complementarity-determining regions (CDRs). “Substantially” refers to the fact that humanized antibodies typically retain at least several donor framework residues (i.e., of non-human parent antibody from which CDRs are derived).

Methods for producing chimeric, primate, primatized®, humanized and human antibodies are well known in the art. See, e.g., U.S. Pat. No. 5,530,101, issued to Queen et al.; U.S. Pat. No. 5,225,539, issued to Winter et al.; U.S. Pat. Nos. 4,816,397 and 4,816,567, issued to Boss et al. and Cabilly et al. respectively, all of which are incorporated by reference in their entirety.

The selection of human constant regions may be significant to the therapeutic efficacy of the subject anti-CG antibody. In a preferred embodiment, the subject anti-CG antibody will comprise human immunoglobulin, gamma-1 (IgG₁), or gamma 3 (IgG₃) constant regions and, more preferably, human IgG₁ constant regions.

Methods for making human antibodies are also known and include, by way of example, production in SCID mice, and in vitro immunization.

The subject anti-CG antibodies can be administered by various routes of administration, typically parenteral. This is intended to include intravenous, intramuscular, subcutaneous, rectal, vaginal, and administration with intravenous infusion being preferred.

The anti-CG antibody will be formulated for therapeutic usage by standard methods, e.g., by addition of pharmaceutically acceptable buffers, e.g., sterile saline, sterile buffered water, propylene glycol, and combinations thereof.

The present invention contemplates an antibody or antibody fragment which recognizes and binds to a CG amino acid sequence.

Effective dosages will depend on the specific antibody, condition of the patient, age, weight, stage of disease, or any other treatment.

Such administration may be effected by various protocols, e.g., weekly, bi-weekly, or monthly, depending on the dosage administered and patient response. Also, it may be desirable to combine such administration with other treatments.

2. Chemical Libraries

Agents that are assayed by these methods can be randomly selected or rationally selected or designed. As used herein, an agent is said to be randomly selected when the agent is chosen randomly without considering the specific sequences involved in the association of CG alone or with its associated substrates, binding partners, etc. An example of randomly selected agents is the use of a chemical library or a peptide combinatorial library, or a growth broth of an organism.

The agents of the present invention may be, as examples, peptides, small molecules, mimetics, vitamin derivatives, as well as carbohydrates. A skilled artisan can readily recognize that there is no limit as to the structural nature of the agents of the present invention.

3. Peptide Synthesis

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production of polypeptides using solid phase peptide synthesis is necessitated if non-nucleic acid-encoded amino acids are to be included.

IX. Uses for Agents that Modulate at Least One Activity of CG, FSH, the FSH/FSHR complex or the CG/LHR Complex

The proteins and nucleic acids of the invention, such as the proteins or polypeptides containing an amino acid sequence of CG, LHR, FSH or FSHR are involved in the modulation of the activities of gonadotropins. Agents that modulate (i.e., up and down-regulate) the expression of CG, FSH, a CG activity, a FSH activity or agents, such as agonists and antagonists respectively, of at least one activity of CG or FSH or their respective receptors may be used to modulate biological and pathologic processes associated with the function and activity of CG or FSH.

Agent contemplated by the present invention include natural and synthetic chemicals of any size. A synthetic or natural chemical may interact with exoloops and modulate them. The chemical compound may contain part of whole of exoloop 1, 2 or 3, or a combination thereof, and modulate the exodomain or endodomain. For example, such an agent may act as a contraceptive by inhibiting or blocking the activity of the exoloops. Conversely, a compound may induce fertility by stimulating the activity of the exoloops.

As used herein, the subject is preferably a mammal, so long as the mammal is in need of modulation of a pathological or biological process modulated by a protein of the invention. The term “mammal” means an individual belonging to the class Mammalia. The invention is particularly useful in the treatment of human subjects.

Because CG, LHR, FSH and FSHR are involved both directly and indirectly in many gonadotropin related diseases and conditions, one embodiment of this invention is to use the present invention as a method of diagnosing a gonadotropin related diseases and conditions. Diagnostic tests for gonadotropin related diseases and conditions may include the steps of testing a sample or an extract thereof for the presence of CG nucleic acids (i.e., DNA or RNA), oligomers or fragments thereof.

This invention also relates to methods of treating gonadotropin related diseases and conditions. This treatment may be achieved by inhibiting or modulating changes in the CG/LHR mechanism or FSH/FSHR mechanism by controlling the binding of CG or FSH to the exoloop 1, exoloop 2 or exoloop 3 of the LHR and FSHR. When phenyl glycoxal is attached to the three arginine residues, this peptide exhibits very high affinity binding to hCG.

The agents of the present invention can be provided alone, or in combination with other agents that modulate a particular pathological process. As used herein, two (or more) agents are said to be administered in combination when the two agents are administered simultaneously or are administered independently in a fashion such that the agents will act contemporaneously.

The agents of the present invention can be administered via parenteral, subcutaneous, intravenous, intramuscular, intraperitoneal, transdermal or buccal routes. Alternatively, or concurrently, administration may be by the oral route. The dosage administered will be dependent upon the age, health, and weight of the recipient, kind of concurrent treatment, if any, frequency of treatment, the nature and stage of disease, and the nature of the effect desired.

The present invention further provides compositions containing one or more agents which modulate expression or at least one activity of a protein of the invention. While individual needs vary, determination of optimal ranges of effective amounts of each component is within the skill of the art. Typical dosages of the active agent which mediate CG activity comprise from about 0.01 mM to 100 mM.

In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, (e.g., ethyl oleate or triglycerides). Aqueous injection suspensions may contain substances which increase the viscosity of the suspension include, for example, sodium carboxymethyl cellulose, sorbitol and/or dextran. Optionally, the suspension may also contain stabilizers. Liposomes and other non-viral vectors can also be used to encapsulate the agent for delivery into the cell.

The pharmaceutical formulation for systemic administration according to the invention may be formulated for enteral, parenteral, or topical administration. If indicated, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled or immediate release forms thereof.

In practicing the methods of this invention, the compounds of this invention may be used alone or in combination, or in combination with other therapeutic or diagnostic agents. In certain preferred embodiments, the compounds of this invention may be co-administered along with other compounds typically prescribed for these conditions according to generally accepted medical practice. For example, the compounds of this invention can be administered in combination with other therapeutic agents (e.g., tamoxifen) for the treatment of gonadotropin related disorders and conditions, as well as with other contraceptives for the prevention of pregnancy.

X. Cis-Activation and Trans-Activation

A new mechanism of control of hormone action has been discovered. The hormone signal can be manipulated or prevented using cis- or trans-activation of a receptor by a hormone receptor complex.

The follicle stimulating hormone receptor (FSHR) binds FSH and activates two distinct effectors, adenylyl cyclase to generate cAMP and phospholipase c to produce inositol phosphate and diacylglycerol. The FSHR may mutate into two types of mutants. It was discovered that one mutant is incapable of binding hormone (i.e., the nonbinding mutant). The other mutant is capable of hormone binding but incapable of activating effectors (i.e, the nonactivating mutant). Some of these two types of mutants co-expressed in a cell are capable of binding hormone and activating effectors. In addition, a receptor complexed with a hormone is capable of activating neighboring unoccupied receptors (trans-activation), in addition to activating itself (cis-activation). The trans-activating pairs can successfully activate adenylyl cyclase to produce cAMP. Other effectors contemplated by the present invention include, but are not limited to, phospholipase C. There my be two distinct mechanisms of activating the two different effectors (i.e., adenylyl cyclase and phospholipase c). These mechanisms have implications for the treatment of inherited disorders of glycoprotein hormone receptors.

A liganded LHR exo-domain can trans-activate the endo-domain of other unliganded LHRs (see Example S herein). It was previously not understood how a hormone receptor could generate two or more signals, such as LHR is capable of activating two enzymes, adenylyl cyclase and phospholipase C, and to generate two distinct signal pathways.

Trans-activation offers a mechanism for a liganded hormone receptor to cis-activate itself and generate a signal. Subsequently, it could trans-activate other receptor molecules for multiple signal generation, yet each receptor interacting with only one G protein to generate a signal at a time. This mechanism would allow one liganded receptor to generate multiple signals without a receptor interacting with multiple G proteins at a time. It could modulate receptor desensitization and phosphorylation.

The ratio of LHR^{+hcG/-cAMP} and LHR^{-hCG} for cAMP rescue is especially important, because trans-activation is likely dependent on the ratio of LHR^{+hCG/-cAMP} and LHR^{-hCG}. Too many LHR^{+hCG/-cAMP} could jam LHR^{hCG}, thus becoming unproductive.

Trans-activation occurs regardless of the hormone binding ability of the unliganded receptor, because the rescued, unliganded LHRs are either incapable or partially capable of hormone binding. For example, LHR^{L29A} and LHR^{153A} are partially active and can more effectively induce cAMP production by trans-activation than by cis-activation. The efficiency of trans-activation varies dependent on the nature of mutations in LHR^{-hCG}. For example, the location of the mutation and the substituting amino acids may effect the efficiency of trans-activation. In particular, the receptors with a mutation near the hinge region and therefore close to the endo-domain cannot be trans-activated. This is likely because the liganded exo-domain of a receptor may have problems reaching such mutated sites and replacing them. It is also possible that the mutations near the hinge region may be irreplaceable due to the hinge region's crucial role in modulating the signal generation. Another possibility is that the mutations close to the hinge region constrain the flexibility of the junction between the exo-domain and endo-domain.

Regardless of whether the successful pairing of coexpressed LHR^{+hCG/-cAMP} and LHR^{hCG} was transiently interacted or stably associated as a dimer, this intermolecular trans-activation is provides and explanation of the mechanisms of receptor activation. A hormone receptor on the cell surface is activated upon binding its cognate hormone, an implication of intra-molecular activation. Similarly, a dimeric receptor complex is activated when the complex interacts with two hormone molecules as shown by the crystal structure of the metabotropic glutamate receptor. The underlying mechanisms for trans-activation of a monomeric receptor and a dimeric receptor may be different. For example, monomeric receptors are more likely to collide and trans-activate, whereas dimeric receptors need to make a specific interaction before trans-activation.

LHR is a crucial component of human reproduction. The present invention sets forth how heterozygotes of two defective mutant LHRs could be reproductive and pass the genes onto next generation and promotes the understanding of mechanisms of mutant receptors and introduces different therapeutic approaches to those mutants such as complementation, rather than replacement of a defective receptor.

### EXAMPLE 1

In order to determine the roles of the N-terminal region in hormone binding and signal generation, short S⁰-K⁴⁰ sequence of the FSHR exodomain were examined. The region not only interacts with FSH, particularly the 13 subunit, but also is involved in modulating signal generation. Human FSH and FSH subunits were purchased from the National Hormone and Pituitary Program. Denatured FSH was prepared by boiling the hormone in 8M urea for 30 mm. Rabbit anti FSHβ sera and rabbit anti FSH13 sera were kindly provided by Dr. James Dias. Anti-rabbit IgG conjugated with peroxidase was purchased from Pierce. Peptide mimics including wild type peptide corresponding to the S⁹-K40 sequence (FSHF⁹⁻⁴⁰) and a photoactivable peptide containing Bpa in place of F¹³ (FSHR^{9-40F13Bpa}) were synthesized by Genemed Synthesis (San Francisco, Calif.) and purified on a Vydac C₁₈ HPLC column using solvent gradient from 100% of 0.1% trifluoroacetic acid in water to 20% of 0.1% trifluoroacetic acid in water and 80% 1-propanol.

Mutagenesis and Functional Expression of Fsh Receptors

Mutant FSHR cDNAs were prepared in the pSELECT vector using the Altered Sites Mutagenesis system (Promega), sequenced on a Beckman CEQ 2000XL capillary sequencer, subcloned into pcDNA3 (Invitrogen), and sequenced again to verify mutation sequences. This procedure does not involve polymerase chain reaction and therefore, does not have its infidelity problems. Wild type and mutant receptor constructs were transfected into HEK 293 cells by the calcium phosphate method as previously described. Stable cell lines were established in minimum essential medium containing 10% horse serum and 500 μg/ml of G418. These cells were used for hormone binding, cAMP production. All assays were carried out in duplicate and repeated 4-5 times, and means±S.D. were calculated.

¹²⁵I-FSH Binding and Intracellular cAMP Assay

Stable cells were assayed for ¹²⁵I-FSH binding in the presence of 100,000 cpm of ¹²⁵I-FSH and increasing concentrations of unlabeled FSH. The Kd values were determined by Scatchard plots. For intracellular cAMP assay, cells were washed twice with Dulbecco's modified Eagle's medium and incubated in the medium containing 0.1 μg/ml isobutylmethylxanthine for 15 mm. Increasing concentrations of FSH were then added and incubation was continued for 45 mm at 37° C. After removing the medium, the cells were rinsed once with fresh medium without isobutylmethylxanthine, lysed in 70% ethanol, freeze-thawed in liquid nitrogen, and scraped. After pelleting cell debris at 16,000×g for 10 minutes at 4° C., the supernatant was collected, dried under vacuum and resuspended in vacuum and resuspended in 10 μl of cAMP assay buffer (Amersham). cAMP concentrations were determined with an ¹²⁵I-cAMP assay kit (Amersham) following the manufacturer's instructions and validated for use.

¹²⁵I-FSH Binding to Solubilized FSHR

Transfected cells were washed twice with ice cold 150 mM NaCl, 20 mM HEPES, pH 7.4 (buffer A). Cells were scraped on ice, collected in buffer A containing protease inhibitors (1 mM phenylmethylsulfonyl fluoride, 5 mM N-ethylmaleimide, and 10 mM EDTA), and pelleted by centrifugation at 1300×g for 10 minutes. Cells were resuspended in 0.6 ml of buffer A containing 1% NP-40, 20% glycerol, and the above protease inhibitors (buffer B), incubated on ice for 15 mm, and diluted with 5.4 ml of buffer A containing 20% glycerol plus the protease inhibitors (buffer C). The mixture was centrifuged at 100,000×g for 60 minutes. The supernatant (500 μl) was mixed with 100,000 cpm of ¹²⁵I-FSH and 6.5 μl of 0.9% NaCl and 10 mM Na₂HPO₄ at pH 7.4 containing increasing concentrations of unlabeled FSH. After incubation for 12 hours at 4° C., the solution was thoroughly mixed with 250 μl of buffer A containing bovine γ-globulin (5 μg/ml) and 750 μl of buffer A containing 20% polyethylene glycol 8000. After incubation for 10 minutes at 4° C., samples were pelleted at 1300×g for 30 mm and supernatants removed. Pellets were resuspended in 1.5 ml of buffer A containing 20% polyethylene glycol 8000, centrifuged, and counted for radioactivity.

Derivatization and Radioiodination of Peptides

In the dark, 30 μg of receptor peptides in 40 μl of 0.1 M sodium phosphate (pH 7.5). The mixture was incubated for 30 minutes at 25° C. The following were added to the derivatization mixture: 1 mCi of Na ¹²⁵I-iodine in 10 μl of 0.1 M NaOH and 7 μl of chloramine-T (1 mg/ml) in 10 mM Na₂HPO₄, pH 7.4. After 20 seconds, 7 μl of sodium metabisulfite (2.5 mg/ml) in 10 mM Na₂HPO₄, pH 7.4, was introduced to terminate radioiodination. Derivatized and radioiodinated ABG-¹²⁵I-FSHR⁹⁻³⁸ solution was mixed with 60 μl of 16% sucrose solution in PBS and fractionated on Sephadex Superfine G-10 column (0.6×15 cm) using PBS.

Photoaffinity Labeling of FSH

The following solutions were sequentially introduced to siliconized glass tubes: 20 μl of 0.9% NaCl and 10 mM Na₂HPO₄, pH 7.4 (PBS), 10 μl of FSH in PBS, and 10 μl of ¹²⁵I-FSHR^{9-40F13Bpa} in PBS. Competitive inhibition experiments were carried out as described for the photoaffinity labeling experiments, except that 10 μl instead of 20 μl of PBS was introduced to each tube and the mixture was incubated with 10 μl of increasing concentrations of nonradioactive receptor peptides. The mixtures were incubated at 37° C. for 90 minutes in the dark, irradiated with a Mineralight R-52 UV lamp for 3 minutes, and solubilized in 2% SDS, 100 mM dithiothreitol and 8 M urea. The samples were electrophoresed on 8-12% polyacrylamide gradient gels. Gels were dried on filter paper and exposed to an imaging screen overnight, which was scanned on a phosphoimager (Molecular Dynamics).

Deglycosylation

The FSH α and β subunits co-migrate on SDS-PAGE. To separate them on the gel FSH was deglycosylated with PNGase F and after it was photoaffinity labeled. Enzymatic cleavage was done by incubation of the labeled FSH complex with 20 or 50 units of PNGase F (New England BioLabs, Inc., MA) in 40 μl for 18 hours at 37° C. The samples were solubilized in SDS under the reducing condition and electrophoresed on 15% gel containing 9 M urea.

Immunoblot of FSH Subunits

Separated proteins were blotted onto 0.2 μm nitrocellulose membrane as previously described. Membranes were treated for 1 hour with 5% blocking buffer (25 mM Tris-HCl, 1.4 M NaCl, 5% Nonfat dry milk, 0.2% Sodium azide, 1% NP4O, pH 7.4) and incubated with polyclonal anti-FSH, anti-FSH α and β antibodies (dilution 1:2000 and 1:3500 each in blocking buffer) for 1 hour at room temperature. Membranes were washed three times (5 minutes each) with the blocking buffer and incubated with anti-rabbit peroxidase-conjugated IgG (dilution 1:5000 in the blocking buffer) for 1 hour at room temperature. Membranes were washed three times (5 minutes each) with the blocking buffer, twice (5 minutes each) with 25 mM Tris-HCl, pH 7.4. Membranes were incubated in staining solution (0.05% 3,3′-diaminobenzidine, 0.02% CoCl₂, 0.03% H₂O₂) until bands became visible.

Ala Scanning of the S⁹-E³³Sequence

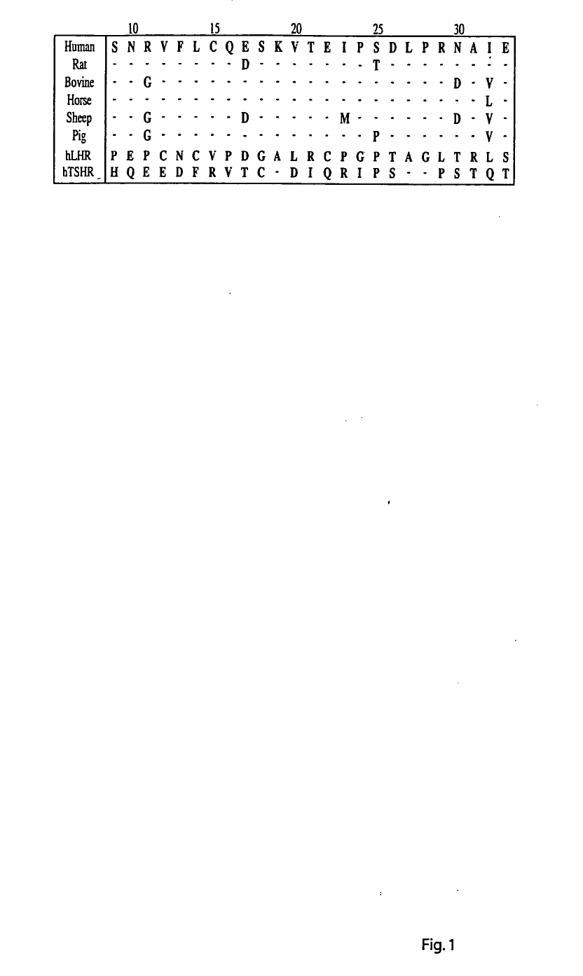

The S⁹NRVFLCQESKVTEIPSDLPRNAIE³³ (SEQ ID NO: 1) sequence of human FSHR is highly conserved among species, but is diverse among the glycoprotein hormone receptors (FIG. 1). As a first step to identify important residues near the N-terminus, each amino acid of the sequence was individually substituted with Ala. This sequence is diverse among the glycoprotein hormone receptors (FIG. 1), although these receptors share a high overall homology and structural similarity. In contrast, the FSHR sequence is highly conserved among species, an indication of its importance.

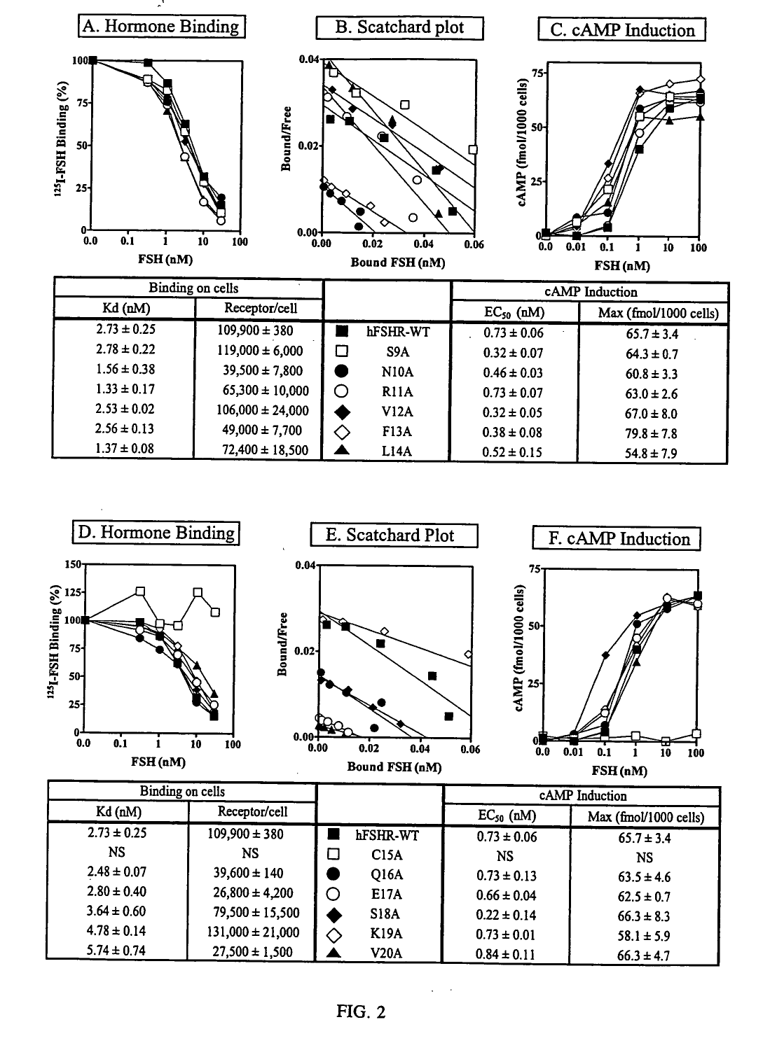

HEK 293 cells were stably transfected with mutant receptor plasmids and selected for stably expressing individual mutant receptors. These cells were assayed for ¹²⁵I-FSH binding and FSH dependent cAMP induction. Ala substitutions for S⁹, N¹⁰, R¹¹, V¹², F¹³ and L¹⁴ improved FSH binding (FIGS. 2A and 2B), FSH-dependent cAMP induction (FIG. 2C) or both. The Kd values of FSHR^{N10A}, FSHR^{R11A} and FSHR^{L14A} were lower than the wild type value, as were the EC₅₀ values of FSHR^{S9A}, FSHR^{N10A}, FSHR^{V12A}, FSHR^{F13A} and FSHR^{L14A} (FIG. 2). Ala substitutions for Q¹⁶, E¹⁷, K¹⁹ and V²⁰ did not impact the EC₅₀ values and maximal cAMP induction (FIG. 2F), and the mutants' Kd values were similar to or somewhat higher than the wild type value (FIG. 2D and 2E). In contrast, the S¹⁸A substitution resulted in a considerably lower EC₅₀ value despite a higher Kd value. These results show an improved cAMP induction in spite of a lower hormone binding affinity, and suggest an interesting and potentially crucial role of S¹⁸ in modulating signal generation.

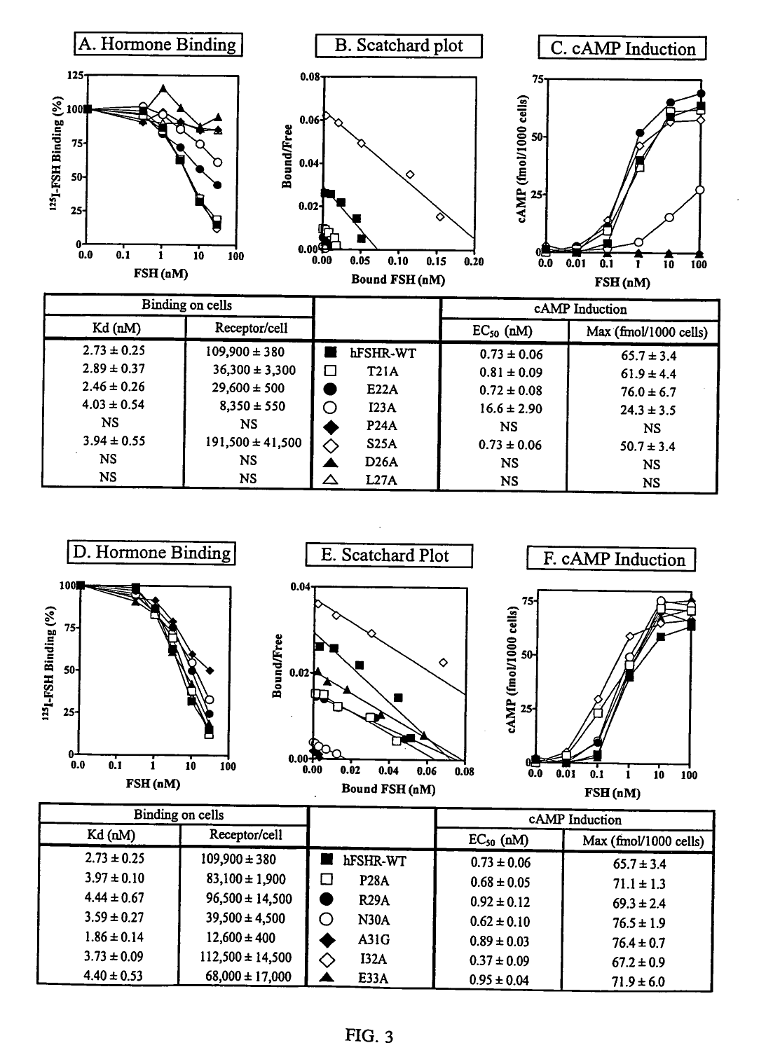

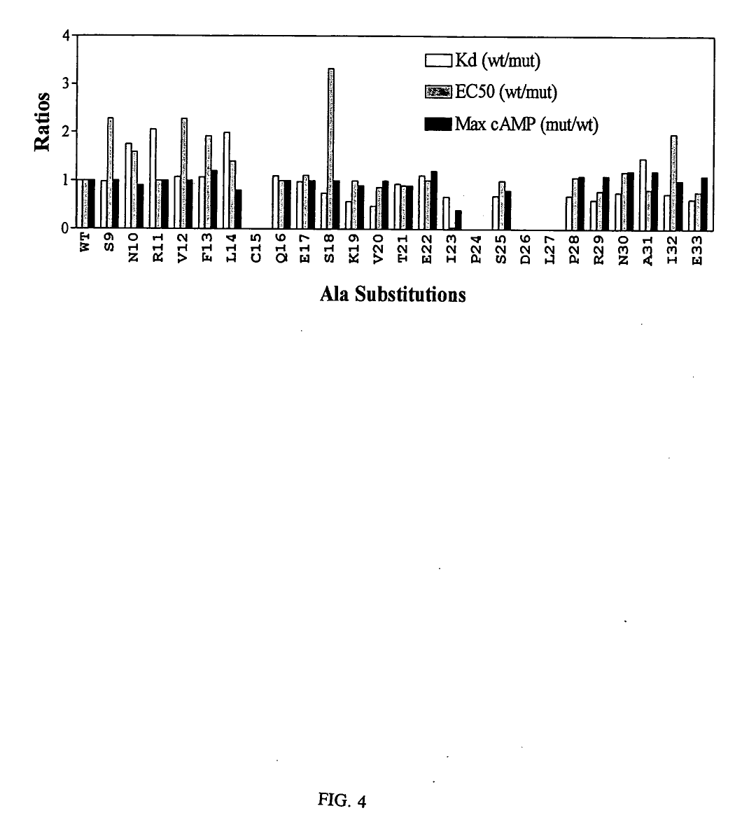

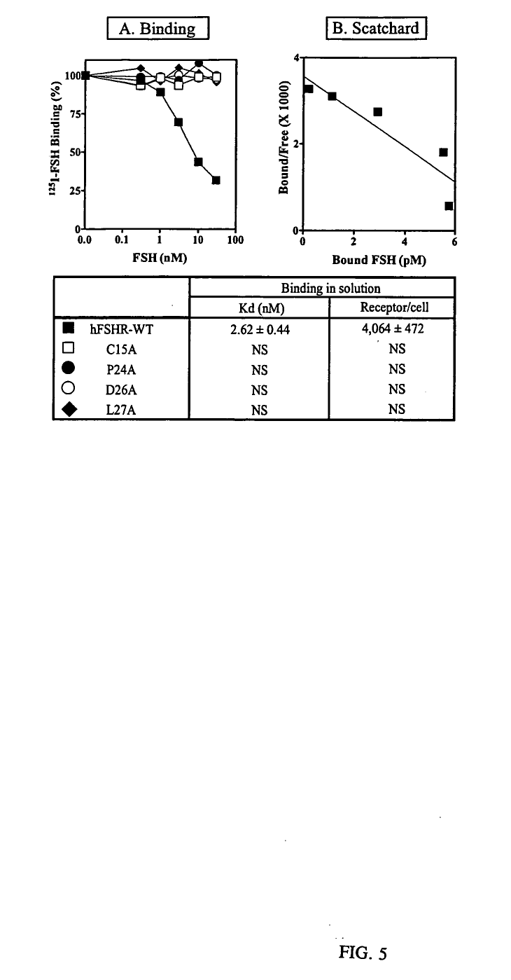

Ala substitution for T²¹ E²² or S²⁵ did not significantly impact hormone binding or cAMP induction (FIG. 3). Conversely, the I²³A substitution partially impaired the cAMP induction with a 23 fold higher EC₅₀ value and 2.6 fold lower maximal cAMP level. The P²⁴A, D²⁶A and L²⁷A substitutions completely abrogated hormone binding and therefore, cAMP induction, suggesting the importance of these residues and this region. The P²⁸A, R²⁹A, N³⁰A, A³¹G I³²A and E³³A substitutions did not dramatically impact the Kd and EC₅₀ values or the maximal cAMP production (FIG. 3). These results, taken together, show several distinct effects of Ala substitutions as shown in the summary bar graph (FIG. 4). C¹⁵A, P²⁴A, D²⁶A and L²⁷A abolished hormone binding. The nonbinding mutants were incapable of binding the hormone or trapped in cells. The binding assay for receptors solubilized in NP-40 showed that FSH did not bind to any of the C¹⁵A, P²⁴A, D²⁶A and L²⁷A mutants (FIG. 5), indicating that they are incapable of hormone binding. In contrast to these nonbinding mutations, N¹⁰A, R¹¹A and L¹⁴A improved hormone binding. On the other hand, I²³A impaired cAMP induction by dramatically increasing the EC₅₀ value. Remarkably, S⁹A, V¹²A, F¹³A, S¹⁸A and I²³A reduced the EC₅₀ value by 2-3 fold, while maintaining or slightly enhancing the maximum cAMP induction level. These results suggested the importance of this region of the receptor in hormone binding and cAMP induction, and raised a question as to whether this region directly interacts with the hormone or indirectly impacts the global structure of the receptor.

Binding and Photoaffinity Labeling of FSH

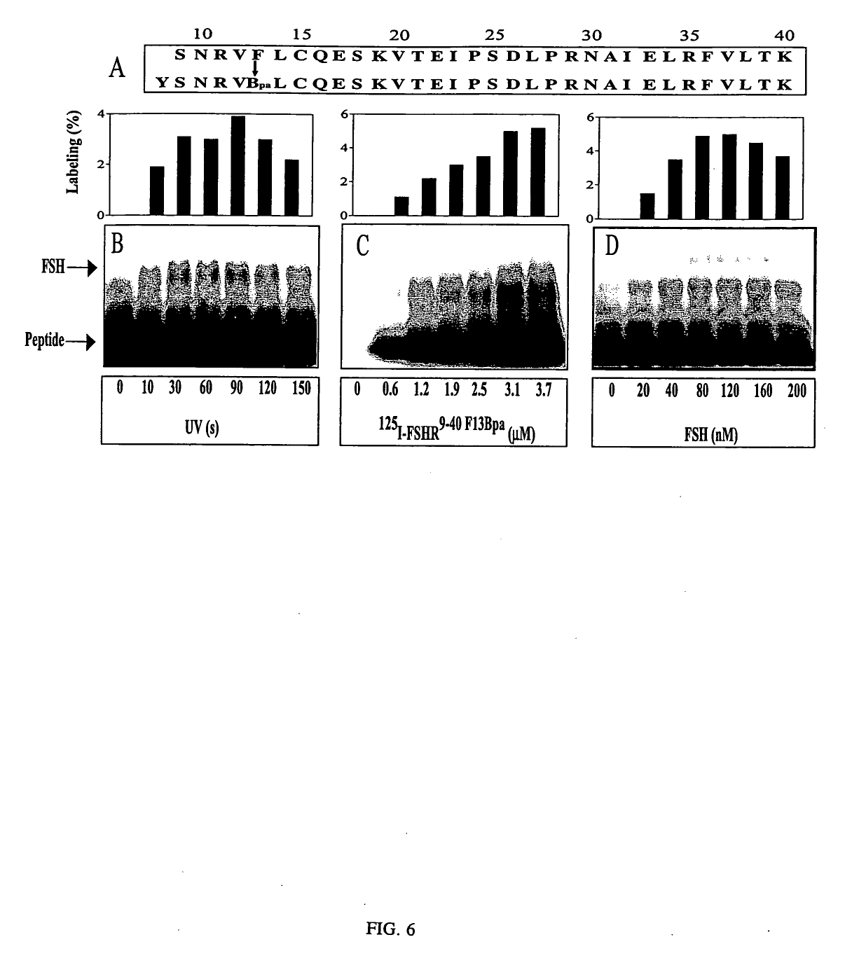

To examine these two general possibilities a peptide mimic corresponding to the receptor sequence of S⁹NRVFLCQESKVTEIPSDLPRNAIELRFVLTK⁴⁰ (SEQ ID NO: 2) was synthesized, FSHFR⁹⁻⁴⁰ (FIG. 6A). A Tyr residue was attached to the N-terminus for radioiodination and the N-terminus was acetylated, while the C-terminus amidated. F¹³ was substituted with benzoyl phenylalanine (Bpa) for photoaffinity labeling. The ketone moiety of the Bpa group can be activated with UV at >350 nm, and is capable of reacting with unreactive α-CH bonds of amino acids. To determine whether the resulting peptide, ¹²⁵I-FSHR^{9-40F13Bpa}, could bind and photoaffinity-label FSH, it was incubated with FSH and irradiated with UV for increasing time periods. Samples were solubilized in SDS under reducing conditions and then electrophoresed. The autoradiographic phosphoimage of the gel (FIG. 6B) revealed labeling of the FSH band. The autoradiograph clearly shows that the two subunits of human FSH comigrated. The band was not labeled when the sample was not irradiated with UV, suggesting the requirement for UV irradiation. The extent of the labeling was dependent on the irradiation time, reaching maximum labeling after 30 seconds irradiation. The result shows that the labeling is saturable.

To determine whether the labeling was specific between the receptor peptide and FSH, the hormone was labeled with increasing concentrations of ¹²⁵I-FSHR^{9-40F13Bpa}, while maintaining FSH at a constant concentration (FIG. 6C). Conversely, increasing concentrations of FSH were labeled with a constant concentration of ¹²⁵-FSHR^{9-40F13Bpa} (FIG. 6D). If the labeling was specific, they should reach a plateau under both conditions. Indeed, the labeling plateaued under both conditions, indicating saturable and specific labeling of more than 50% of FSH.

Labeling Specificity

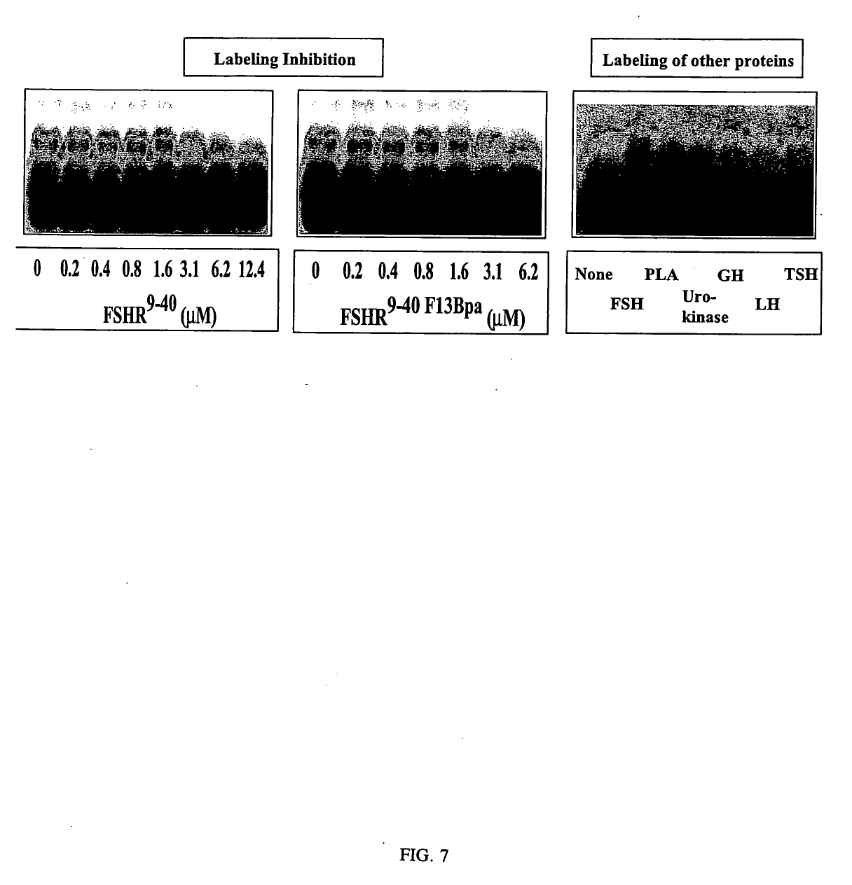

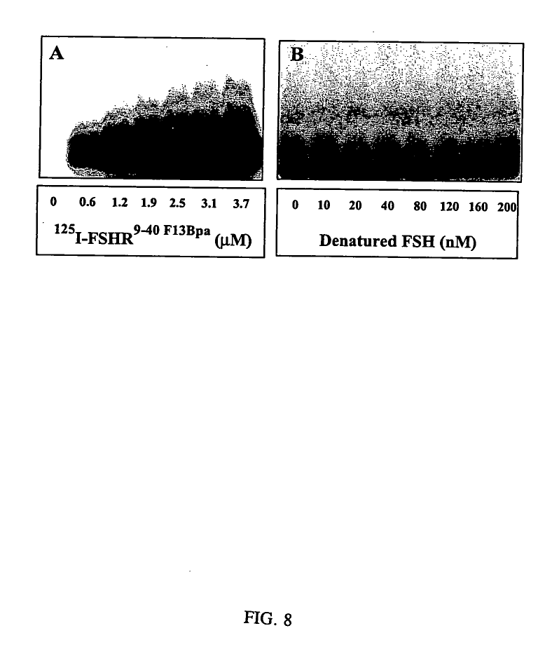

If the labeling was specific as suggested by the results of FIGS. 6B-D, the labeling should be inhibited by nonradioactive peptide and unmodified wild type peptide. Therefore, FSH was incubated with ¹²⁵I-FSHR^{9-40F13Bpa} in the presence of increasing concentrations of nonlabeled wild type peptide (FIG. 7A) and nonradioactive FSHR^{9-40F13Bpa} (FIG. 7B). Increasing concentrations of the peptides inhibited the photoaffinity labeling in a dose dependent manner and eventually, blocked the labeling. To determine the labeling specificity, LH, TSH, growth hormone, phospholipase A and urokinase were subjected to photoaffinity labeling with ¹²⁵I-FSHR^{9-40F13Bpa} (FIG. 7C). None were labeled. Although the photoaffinity labeling was specific for FSH, our data do not show the biological significance of the affinity labeling. To address this concern, a constant amount of denatured FSH was incubated with increasing concentrations of ¹²⁵I-FSHR^{9-40F13Bpa} and treated with UV. Denatured FSH was not labeled at all, despite high concentrations of the peptide (FIG. 8A). Denatured FSH was not labeled when increasing concentrations of denatured FSH were incubated with a constant amount of ¹²⁵I-FSHR^{9-40F13Bpa} and treated with UV (FIG. 8B). FSH was denatured by boiling in 8 M urea for 30 mm, which did not bind to FSHR and induce cAMP production. To test whether the denatured FSH remained in solution, the mixture of radioactively labeled FSH and unlabeled FSH was denatured, and varying volumes of the mixture were transferred to another tubes and the radioactivity was counted. The transfer was quantitative with a 99-100 efficiency, indicating denatured FSH was present in the photoaffinity labeling tube. These results indicate the specificity of the affinity labeling for biologically active FSH.

Labeled FSH Subunit

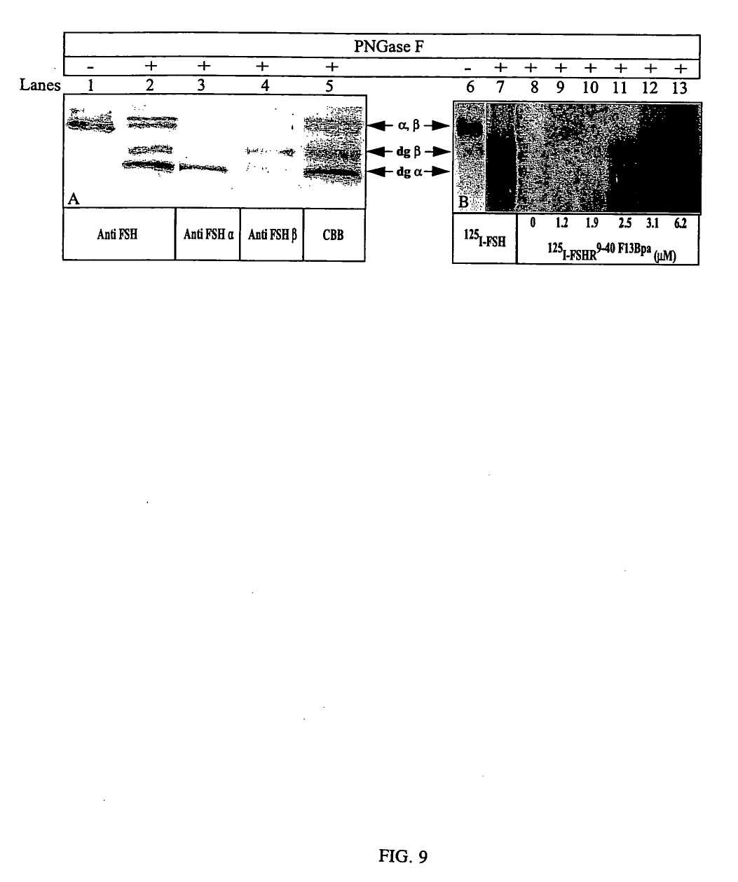

FSH subunits could be separated on SDS-PAGE after deglycosylation with PNGase F (FIG. 9A, lane 2). It can be clearly seen that this procedure allows identification of the labeled upper band. Since the β subunit is larger than the α subunit, the upper band was likely the β subunit. To clarify the identity of the upper band, deglycosylated FSH was electrophoresed and the gel was blotted on nitrocellulose membrane, then probed with antiFSH α and antiFSHβ antibodies. AntiFSH α antibody conspicuously labeled the lower band, whereas the antiFSH β antibody recognized primarily the upper band and faintly the lower band (FIG. 9B). These results show that the lower band represents the FSHα subunit whereas the upper band is the FSHβ subunit. The identity and specificity of the β subunit labeling are underscored by the remarkably contrasting labeling of the FSH α subunit by the FSHR exoloop 3 peptide.

The Ala Scanning results indicate that the S⁹-E³³ sequence of the FSH receptor is important for hormone binding and signal generation. Furthermore, the photoaffinity labeling results show that FSHR^{9-40F13Bpa} photoaffinity labels FSH. Ample evidence is presented to support the specificity of the photoaffinity labeling under rigorous conditions. The labeling is saturable and dependent on the FSH concentration, derivatized ¹²⁵I-FSHR⁹⁻⁴⁰ concentration, UV activation and UV exposure time period. FSHR^{9-40F13Bpa} photoaffinity labels bioactive FSH but not denatured hormone, and the labeling is blocked by nonderivatized wild type peptide. The labeling specificity is further underscored by the fact that the β subunit, but not the α subunit, in FSH was labeled. These results suggest that the N-terminal region of the FSH receptor makes contact with FSH. This is consistent with the observation that the similar region of the LH/CG receptor interacts with hCG.

Computer modeling based on the crystal structure of ribonuclease inhibitor suggests that the 8-9 Leu rich repeats make up the bulk of the exodomain and assume a ⅓ doughnut structure, which provides the primary hormone contact site. Each Leu rich repeat comprises a β strand and α helix connected by a linker, and the β strands make up the concave surface, while the helices form the convex surface. The β strands lining the concave surface are thought to interact with the hormone. Although mutational analyses of the Leu rich repeats revealed that only a few were important for high affinity hormone binding, upon hormone binding, accessibility of these repeat regions becomes limited, suggesting the possibility for more extensive interactions in the concave surface). In addition, the short N-terminal region flanking the Leu rich repeats is crucial for FSH binding, appears involved in a conformational change upon hormone binding, and interacts with hCG. These results are entirely consistent with our observations described in this study.

Although both FSH and hCG can be photoaffinity labeled with their cognate receptor peptides, there is a striking difference in the labeling of the gonadotropins. In the current study, FSH was labeled only at the β subunit, whereas hCG was labeled primarily at the α subunit. These results indicate critical differences in the hormone-receptor interactions of FSH and hCG, in particular at the N-terminal region of the receptors, and underscores the significance of our observations described in this study. Consistent with the differential labeling results, there is no sequence homology in this region of the receptors. This suggests that the region of the receptors is likely a determining factor for the hormone specificity. Such hormone specificity may also be found in the hormones. The crystal structures of hCG and FSH are generally similar with identical folds, but there are several crucial differences including the N- and C-termini and loop 2 of the β subunits. A comparison of the two structures shows considerable flexibility in β1 and β3 loops. In addition, the long loop β2 shows a large degree of conformational flexibility. Some of these sites may reflect the differential labeling, and therefore, it will be interesting to identify such distinct sites and the photoaffinity labeled amino acids of the hormones. It is striking that FSH β was labeled by the N-terminal peptide, compared to labeling of FSH α by the FSHR exoloop 3 peptide. These results reinforce the specificity and validity of the photoaffinity labeling results, and suggest the dynamic nature of the interactions among the exodomain, FSH and endodmain from the initial hormone contact with the exodomain to the signal generation in the ternary complex. It has been questioned whether the quaternary structure of the intercalated subunits is a prerequisite for receptor binding/signal generation, based on recent observations using single chain glycoprotein hormone analogs.

The results that Ala substitution for some N-terminal residues improved hormone binding, cAMP induction or both suggest an interesting possibility that this region is involved in modulating not only hormone binding but also signal generation. The most dramatic improvement is seen in the S¹⁸A substitution, which improved the EC₅₀ value of cAMP induction by 3 fold, as compared with the wild type value. Additionally, the maximum level of cAMP production only slightly increased. These observations indicate that the affinity and maximum level of cAMP induction are distinctly regulated. They suggest that FSH activates FSHR^{S18A} more effectively than the wild type receptor does, which in turn results in better activation of the G protein. The improved EC₅₀ is not related to the hormone binding affinity, as the binding affinity of the mutant is somewhat less than the wild type affinity. These novel observations suggest the possibility that FSHR^{S18A} is more sensitive to hormone binding and is capable of activating the G protein with higher affinity, without significantly impacting the level of activation. Because the exodomain is likely to modulate the endodomain to generate hormone signals at the exoloops, a simple possibility is that the affinity of the modulation at the interface between the exodomain and exoloops is improved in FSHR^{S18A}. Several other Ala substitutions, S⁹A, V¹²A and F¹³A, also showed similar yet less dramatic results. The exclusive photoaffinity labeling of FSH β by the Bpa at the F¹³ position implicates that the region contacts with the β subunit and the resulting complex modulates the signal generation.

Some substitutions for S²⁵⁵ in the hinge region of the LH receptor exodomain lead to constitutive activation of cAMP induction without improving the EC₅₀ value. On the other hand, the Ala substitution for S¹⁸ and the other residues did not constitutively activate the receptor but enhanced the EC₅₀ value of cAMP induction. Therefore, there appear to be distinct mechanisms.

### EXAMPLE 2

Mutagenesis and Functional Expression of Human FSH Receptor

Each mutant human FSHR cDNA was prepared in a pSELECT vector using the non-PCR based Altered Sites Mutagenesis System (Promega), sequenced on a Beckman CEQ 2000XL capillary sequencer and subcloned into pcDNA3 (Invitrogen) as described. After subcloning pcDNA3 the mutant cDNAs were sequenced again. Plasmids were transfected into human embryonic kidney (HEK) 293 cells by the calcium phosphate method. Stable cell lines were established in minimum essential medium containing 10% horse serum and 500 μg/ml of G-418, and then used for hormone binding and cAMP assay. All assays were carried out in duplicate and repeated 4-6 times. Means and standard variations were calculated.

¹²⁵I-FSH Binding and Intracellular cAMP Assay

Human FSH (the National Hormone and Pituitary Program) was radioiodinated as previously described for radioiodination of hCG. Denatured FSH was prepared by boiling in 8M urea for 30 minutes. Stable cells were assayed for ¹²⁵I-FSH binding in the presence of increasing concentrations of nonradioactive FSH. The Kd values were determined by Scatchard plots. Truncated exodomain was solubilized in Nonidet P-40 and assayed for hormone binding as described previously. For intracellular cAMP assay, cells were washed twice with Dulbecco's modified Eagle's media and incubated in the media containing isobutylmethylxanthine (0.1 mg/ml) for 15 minutes. Increasing concentrations of FSH were then added and the incubation was continued for 45 minutes at 37° C. After removing the media, the cells were rinsed once with fresh media without isobutylmethylxanthine, lysed in 70% ethanol, freeze-thawed in liquid nitrogen, and scraped. After pelleting cell debris at 16,000×g for 10 minutes at 4° C., the supernatant was collected, dried under vacuum and resuspended in 10 μl of the cAMP assay buffer, which was provided by the manufacturer. cAMP concentrations were determined with an ¹²⁵I-cAMP assay kit (Amersham) following the manufacturer's instruction and validated for use in our laboratory. Exoloop 3 of FSHR was modeled based on the crystal structure of rhodopsin as a template.

Inositol Phosphate Assay

Stable cells were plated in 12 well plates and grown in inositol free DMEM (Atlanta Biologicals) supplemented with 8% heat-inactivated horse serum and 2 μCi/ml ³H-inositol (N EN) for 48 h to 40-50% confluency. After removing the medium, the cells were incubated in 1 ml of fresh wash buffer consisting of DMEM without inositol and 15 mM HEPES (pH 7.3) for 1 hour at 37° C. This medium was removed and 0.3 ml wash buffer containing 20 mM LiCl was added and incubated for 15 minutes at 37° C. After the cells were stimulated with increasing concentrations of hormone for 30 minutes at 37° C., the incubation was terminated by the removal of medium and the addition of 0.25 ml of 0.6 N HCl to each well. The cells were scraped and transferred into microcentrifuge tubes, and the wells were again washed with 0.25 ml of 0.6 N HCl. The combined washes were treated with 0.9 ml of a mixture of chloroform:methanol (2:1), vortexed and centrifuged at 1000×g for 5 minutes at room temperature. The top aqueous layer, which was free of phospholipids, was removed and the remaining chloroform layer treated with 0.2 ml of methanol:water (1:1), vortexed and centrifuged. This aqueous layer was added to the previous aqueous layer and the samples dried in a vacuum concentrator. The dried samples were redissolved in 0.5 ml of 50 mM Tris-HCl, pH 8 and applied to Dowex AG 1-X8 formate (BIO-RAD) columns. The microcentrifuge tubes were washed twice with 0.5 ml of the same buffer and the washes applied to the columns for a total of 1.5 ml. The columns were sequentially washed with 4.5 ml H₂O and 4.5 ml 60 mM ammonium formate and 5 mM sodium tetraborate to elute the free inositol and the glycerol phosphoinositol. IP₁, IP₂ and IP₃ were sequentially eluted with 4 ml of 0.1 N formic acid in 0.2 M, 0.75 M ammonium formate and 1.1 M ammonium formate, respectively and collected in 1 ml fractions. Aliquots of 200 μl were counted for radioactivity in 1.5 ml of Ultima AF scintillation fluid (Packard). Peak radioactivities were used for the data analysis.

Derivatization and Radioiodination of Peptide

A peptide mimic corresponding to the exoloop 3 sequence of K⁵⁸⁰ VPLITVSKAK⁵⁹⁰ (FSHR exo3) (SEQ ID NO: 3) was synthesized, to which a Tyr residue was attached to the C-terminus for radioiodination. The N-terminus of the peptide was acetylated and the C-terminus amidated. NHS-ABG was synthesized as previously described and freshly dissolved in dimethyl sulfoxide to a concentration of 50 mM and NHS-ABG in 0.1 M sodium phosphate (pH 7.5) to a concentration of 20 mM. These reagent solutions were immediately used to derivatize receptor peptides. In the dark, 10 μl of NHS-ABG was added to 30 mg of receptor peptides in 40 μl of 0.1 M sodium phosphate (pH 7.5). The mixture was incubated with NHS-ABG for 30 minutes at 25° C. The following were added to the derivatization mixture: 1 mCi of Na ¹²⁵I-iodine in 10 μl of 0.1 M NaOH and 7 μl of chloramine-T (1 mg/ml) in 10 mM Na₂ HPO₄, pH 7.4. After 20 sec, 7 μl of sodium metabisulfite (2.5 mg/ml) in 10 mM Na₂ HPO₄, pH 7.4, was introduced to terminate radioiodination. Derivatized and radioiodinated ABG-¹²⁵I-FSHR^{exo3} solution was mixed with 60 μl of 16% sucrose solution in PBS and fractionated on Sephadex Superfine G-10 column (0.6×15 cm) using PBS.

Photoaffinity Labeling of FSH

The following solutions were sequentially introduced to siliconized glass tubes: 20 μl of 0.9% NaCl and 10 mM Na₂ HPO₄, pH 7.4 (PBS), 10 μl of FSH (10 ng/μl) in PBS, and 10 μl of ABG-¹²⁵I-FSHR^{exo3} (10 ng/μl) in PBS. Competitive inhibition experiments were carried out for the photoaffinity labeling experiments. 10 μl of PBS was introduced to each tube and the mixture was incubated with 10 μl of increasing concentrations of nonradioactive receptor peptides. The mixtures were incubated at 37° C. for 90 minutes in the dark, irradiated with Mineralight R-52 UV lamp for 3 minutes as previously described, and solubilized in 2% SDS, 100 mM dithiothreitol and 8 M urea. The samples were electrophoresed on 8-12% polyacrylamide gradient gels. Gels were dried on filter paper and exposed to an imaging screen overnight, which was scanned on a phosphoimager.

Deglycosylation

The FSH αa and β subunits co-migrate on SDS-PAGE. To separate them on the gel FSH was deglycosylated with PNGase F before and after it was photoaffinity labeled. Enzymatic cleavage was done by incubation of the labeled FSH complex with 20 or 50 units of PNGase F (New England BioLabs, Inc., MA) in 40 μl for 18 hours at 37° C. The samples were solubilized in SDS under the reducing condition and electrophoresed on 15% gel containing 9 M urea.

Effects of Ala Substitutions on Production of IP₁, IP₂ and IP₃

FSHR exoloop 3 consists of 11 amino acids, K⁵⁸⁰ VPLITVSKAK⁵⁹⁰ (SEQ ID NO: 4), which are conserved among species except A 589 (FIG. 1). The sequence is also conserved among the glycoprotein hormone receptor family except S⁵⁸⁷ KA⁵⁸⁹ near the C-terminus. The previous Ala scan has demonstrated that exoloop 3 constrains the hormone binding at the exodomain and plays a crucial role in cAMP induction. However, little is known about the mechanism or its role in IP induction. To address these questions the Ala substituents of individual residues, except for the A⁵⁸⁹ G substitution, were stably expressed on HEK293 cells. The cells were assayed for inositol phosphates, IP₁, IP₂, IP₃ and IPₜ. Most of the mutant receptors, except the V⁵⁸¹ A, P⁵⁸² A substitutions, were incapable of inducing IP production in response to FSH (FIG. 2A). In contrast, V⁵⁸¹ A was capable of producing noticeable levels of IP₁ and IP₂ and an insignificant level of IP₃. P⁵⁸² A produced a detectable level of IP₁ but not IP₂ and IP₃. These results raise the question of whether the non-responding mutant receptors were expressed on the cell surface in this study, although they were in the previous study. Therefore, the cells stably transfected with the mutants were assayed for ¹²⁵I-FSH binding as well as FSH dependent cAMP production.

Distinct Effects of Ala Substitutions on Hormone Binding, IP Induction and cAMP Induction

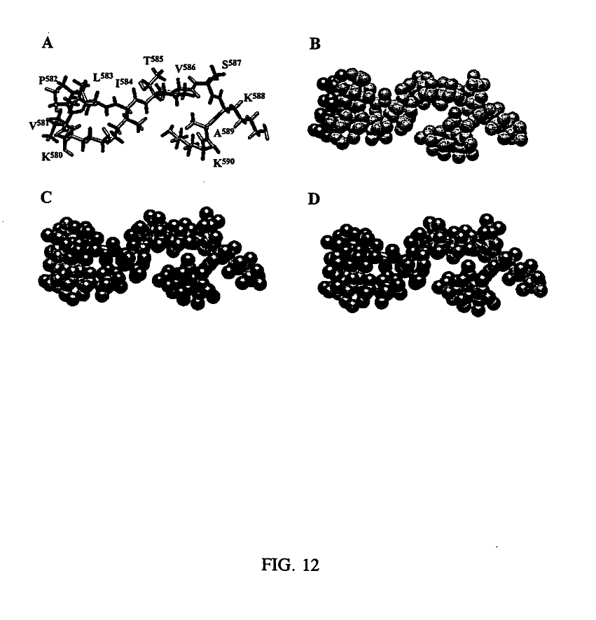

For easy comparison of the data, the ratios of Kd^{wild type/mutant} (Kd^{wt/mut}), maximum IPt^{mut/wt} and maximum cAMP^{mut/wt} were calculated (FIG. 2B). The results show that all of the cells bound the hormone, indicating the surface expression of the mutant receptors. Of interest is that the L⁵⁸³ A and I⁵⁸⁴ A mutations improved the hormone binding affinity by 2-3 fold. This is in striking contrast to the loss of IP induction by most of the mutants except the V⁵⁸¹ A and P⁵⁸² A mutants. On the other hand, the mutational effect is less severe on the activation of adenylyl cyclase to produce cAMP. Most of the mutants were capable of producing some cAMP, although less than the wild type. The three mutants, L⁵⁸³ A, I⁵⁸⁴ A, and K⁵⁹⁰ A, did not produce cAMP. Therefore, the activation of PLCβ is more sensitive to Ala substitution than is the activation of AC and hormone binding. The results also show different mechanisms, in particular the sites, of the PLCb activation, AC activation and hormone binding. We cannot, however, unequivocally dismiss the possibility that the lack of the IP induction was due to the limitation of the detectable Ips. To visualize the difference, exoloop 3 was computer-modeled (FIG. 12A). The results showed the contrasting topography of the sensitive residues for the signal generation and hormone binding. The residues crucial for the PLCβ signal cover most of exoloop 3 except the N-terminal region (FIG. 12B). On the other hand, the residues sensitive to the AC signal are confined in the middle and C-terminus of the exoloop (FIG. 12C). L⁵⁸³ and I⁵⁸⁴ are most sensitive to hormone binding and are located near the middle of the exoloop (FIG. 12D). Their side chains protrude in opposite directions. The sensitive residues appear to be accessible from one side of the exoloop, suggesting the possibility that they might be modulated from the side of the exoloop by the exodomain and/or the hormone. Particularly, L⁵⁸³ and I⁵⁸⁴ are sensitive to all of the three functions: hormone binding, PLCβ activation and AC activation. In addition to L⁵⁸³ and I⁵⁸⁴, K⁵⁹⁰, is important to the activation of PLCβ and AC.

Multiple Mutational Analysis and Specificity of L⁵⁸³, I⁵⁸⁴, K⁵⁹⁰ and P⁵⁸²

The Ala substitution for L⁵⁸³ or I⁵⁸⁴ enhanced the hormone binding affinity by 2-3 fold, but impaired signal generation for IP and cAMP. These two residues have large hydrophobic side chains, which are exposed on the surface according to the computer model (FIG. 12). Hydrophobic side chains are generally incompatible with surface exposure, especially to water molecules. However, surface exposure provides a hydrophobic contact site. To examine the roles of the side chains, L⁵⁸³ and I⁵⁸⁴ were substituted with a panel of amino acids with various side chains: negative or positive, hydroxyl, neutral, ring or aliphatic groups. In addition, the residues were deleted in deletion mutants, which is helpful in assessing the effect of removing the original side chain without introducing a new side chain.

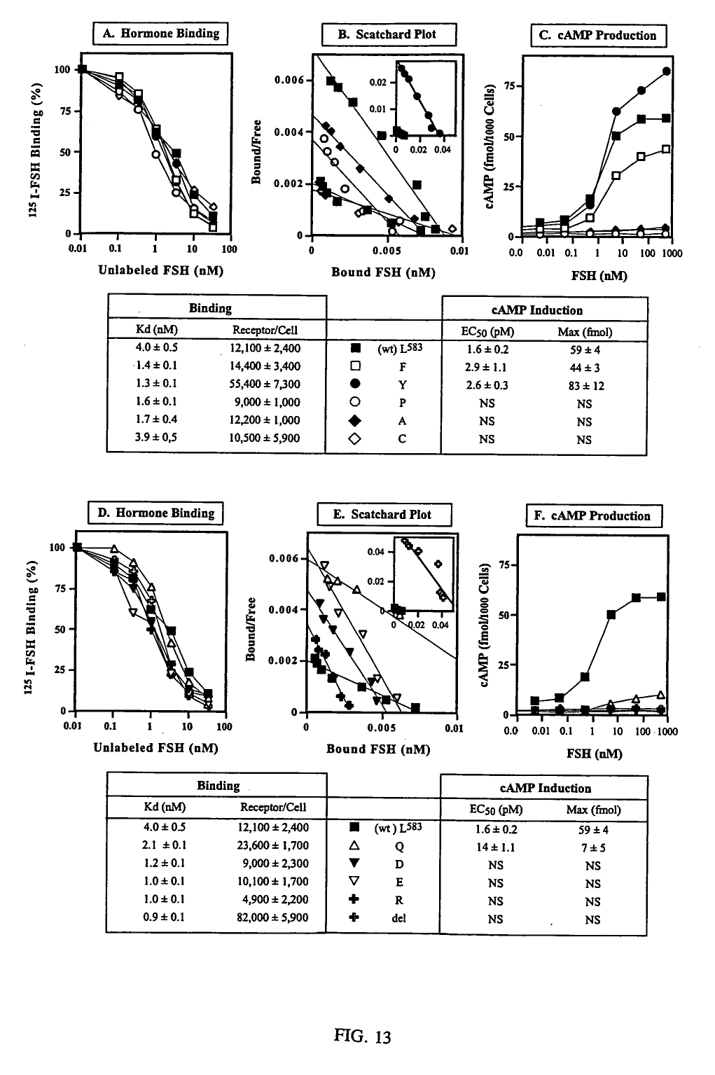

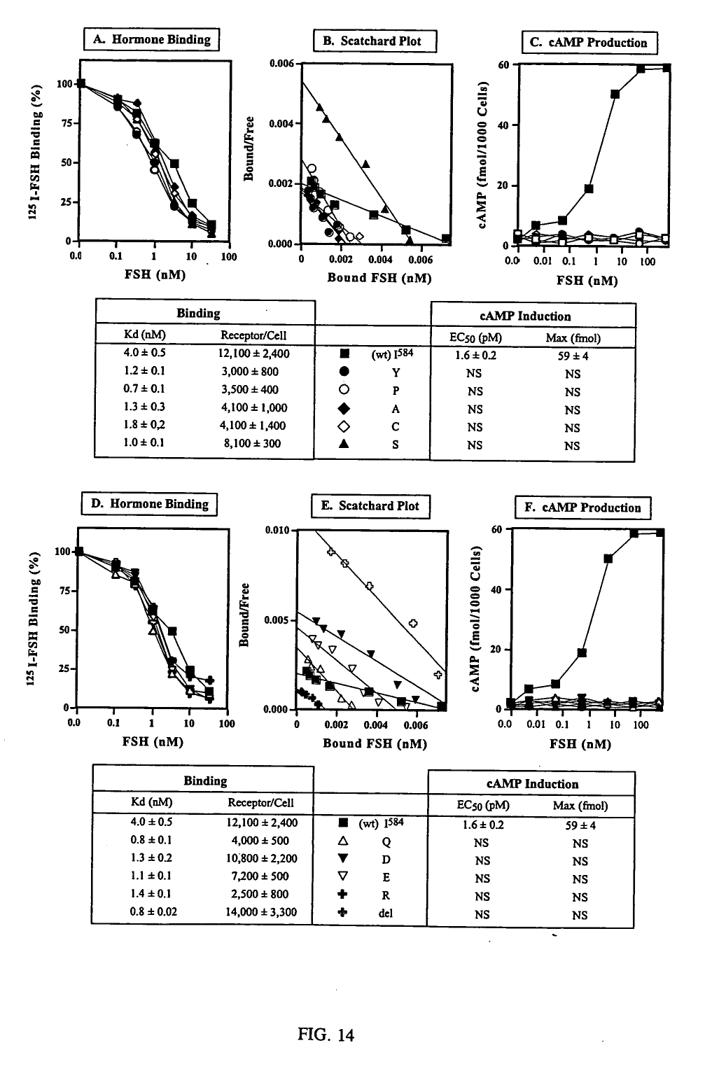

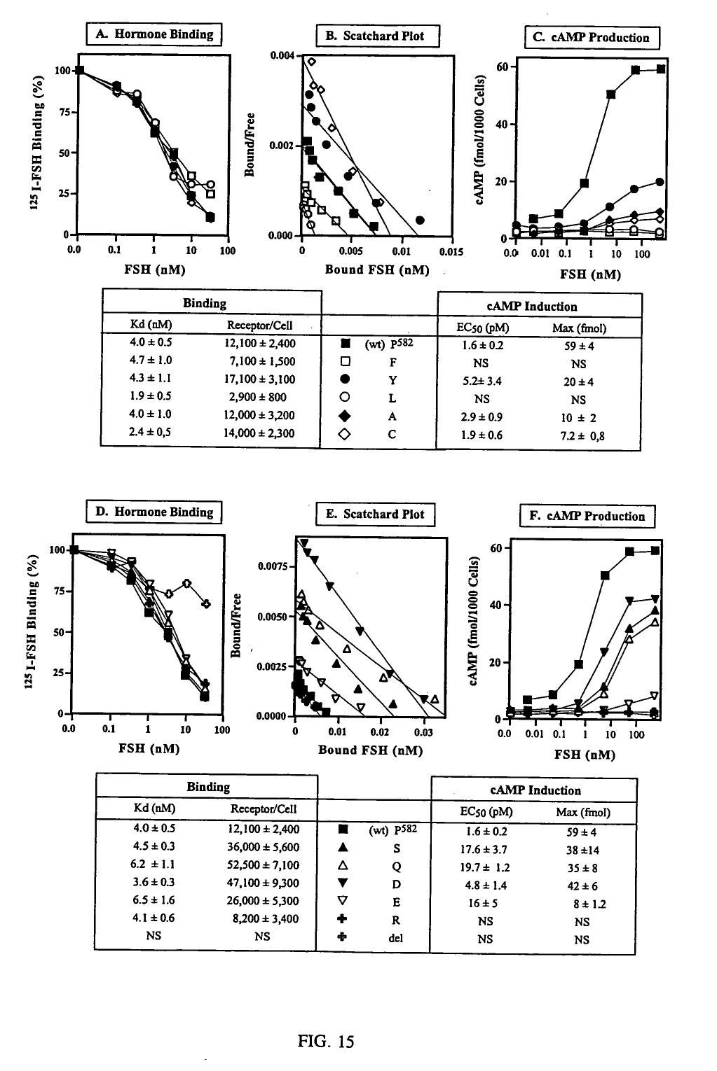

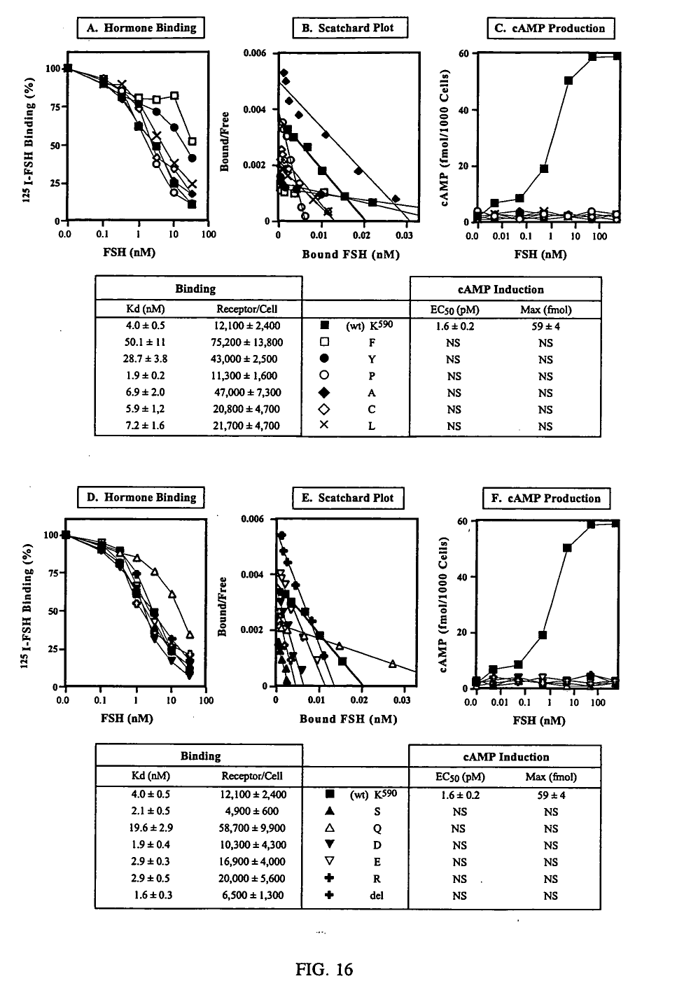

As shown in FIGS. 13A, 13B, 13D and 13E, all of the substitutions of L⁵⁸³ decreased the Kd values, thus improving the binding affinity. Even when L⁵⁸³ was deleted, the affinity improved by more than 4 fold, suggesting that the loss of the L⁵⁸³ side chain contributes to the improved binding affinity. In contrast to the improvement in hormone binding, most of the mutants did not induce noticeable amounts of cAMP (FIG. 13C). Exceptions were the L⁵⁸³ F and L⁵⁸³ Y mutants that induced significant amounts of cAMP with reasonable EC₅₀ values. Besides the L⁵⁸³ F and L⁵⁸³ Y mutants, the L⁵⁸³ Q mutant induced a marginal level of cAMP (FIG. 13F). These results suggest the need of a specific group such as the Leu side chain or a ring group for the AC activation. When the Y, F, A, E, R and deletion mutants were assayed for IPₜ production, only the L⁵⁸³ Y mutant produced a small amount of IPₜ. This result suggests a similarity in the interactions to activate AC and PLCβ. All substitutions for and deletion of I⁵⁸⁴ decreased the Kd values by up to 5 fold (FIG. 14). In contrast to this improved hormone binding, none of the mutants induced cAMP. Therefore, the deletion of I⁵⁸⁴, not the introduction of new side chains, was likely responsible for the improved binding affinity. Furthermore, I⁵⁸⁴ appears to be crucial for cAMP induction (FIG. 14C). To further test these hypotheses, the adjacent P⁵⁸² was substituted with various amino acids (FIG. 15). The mutational effect on hormone binding was diverse and less dramatic. Some mutations decreased the Kd value, whereas others increased it. Several mutants were capable of inducing cAMP, whereas several others failed to induce the second messenger. P⁵⁸² appears to play a role different from those of L⁵⁸³ and I⁵⁸⁴. In addition to P⁵⁸², K⁵⁹⁰ was examined with multiple substitutions. K⁵⁹⁰ is located at the far end of exoloop 3, at the boundary with the transmembrane 7. The Kd values of the substituents varied widely from 1.6 nM to 50 nM as compared with the wild type value of 4 nM, whereas none of the mutants induced significant amounts of cAMP (FIG. 16). Therefore, K⁵⁹⁰ is also essential for activation of AC. In addition, the mutants with C, F, L, Y, A and R substitutions for K⁵⁹⁰ were assayed for production of IP₁, IP₂, IP₃ and IPₜ. None of the mutants induced significant amounts of any IP species.

Photoaffinity Labeling of FSH with Exoloop 3 Peptide

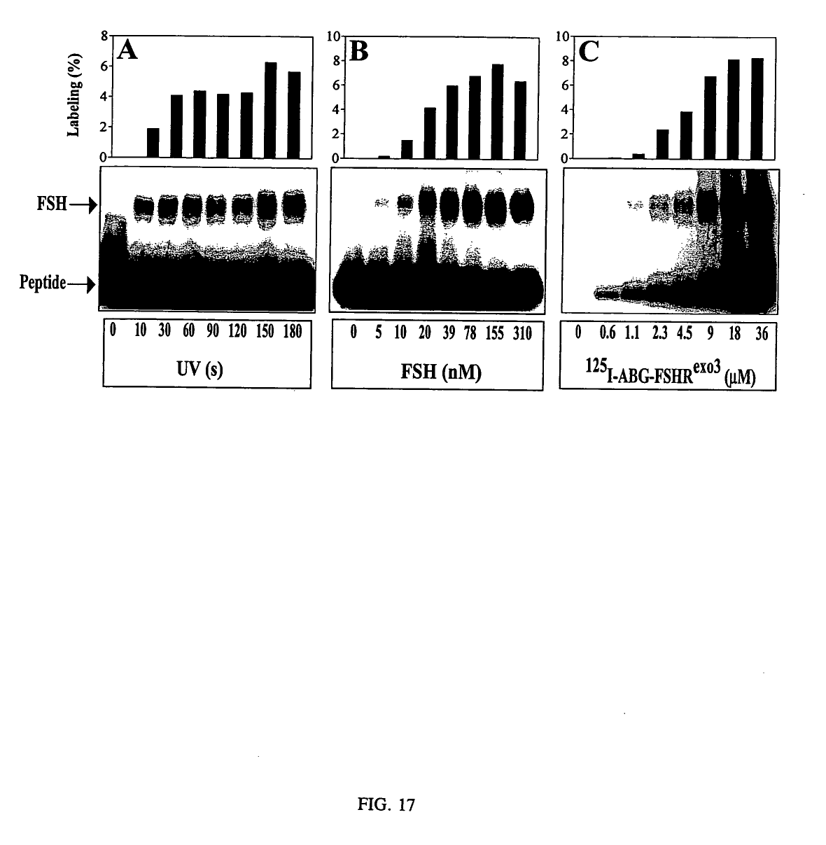

To test the possible interaction of exoloop 3 with the hormone, ¹²⁵I-ABG-FSHR^{exo3}I was incubated with FSH, and irradiated with UV for increasing time periods. Samples were solubilized in SDS under the reducing condition and electrophoresed. The autoradiographic phosphoimage of the gel (FIG. 17A) revealed the labeling of the FSH band. The two subunits of the human FSH preparation comigrate on SDS-PAGE. The band was not labeled when the sample was not irradiated with UV, suggesting the requirement for UV irradiation. The extent of the labeling was dependent on the irradiation time, reaching the maximum after 60 seconds of irradiation. The results show that the labeling is saturable.

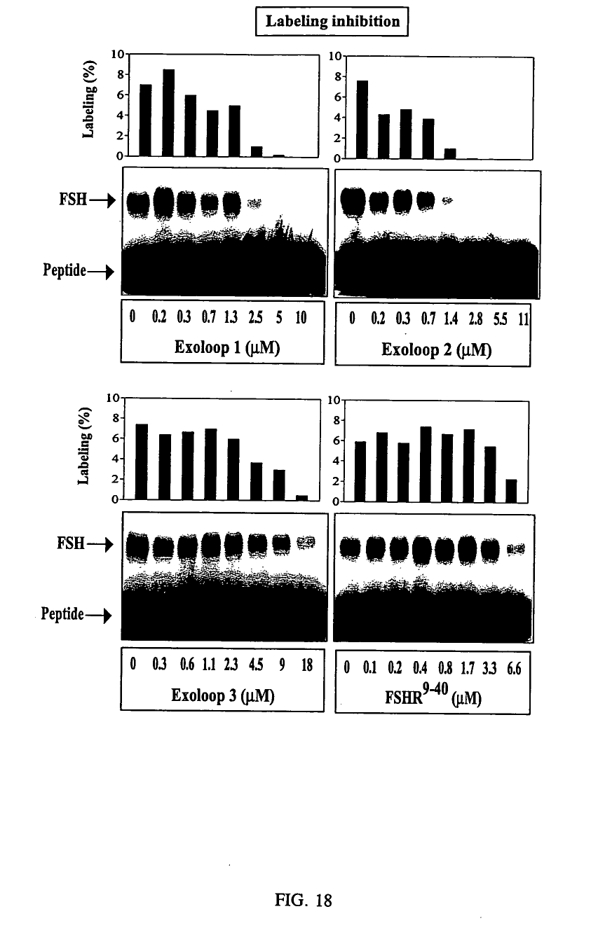

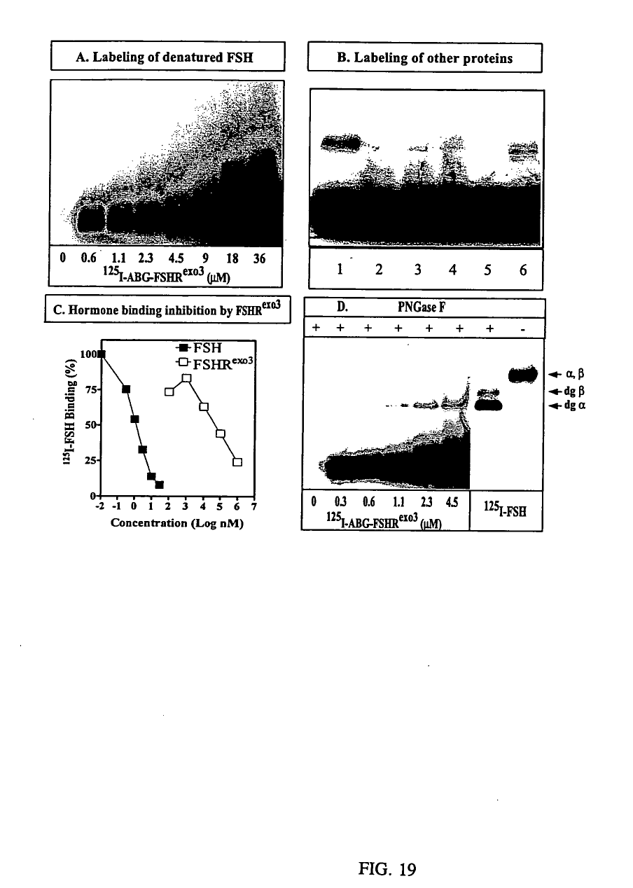

To determine whether the nature of the labeling, increasing concentrations of the hormone were labeled with a constant amount of ¹²⁵I-ABG-FSHR^{exo3}I (FIG. 17B). Conversely, increasing concentrations of ¹²⁵I-ABG-FSHR^{exo3}I were used to label a constant amount of FSH (FIG. 17C). The labeling plateaued under both conditions, indicating saturable labeling. To examine the relationship of the labeling with other exoloops and receptor peptide, FSH was incubated with in the presence of increasing concentrations of unlabeled FSHR peptides corresponding to exoloops 1, 2 and 3 as well as the N-terminal sequence S⁹-K⁴⁰, FSHR⁹⁻⁴⁰, which is known to interact with FSH. Increasing concentrations of the peptides inhibited the photoaffinity labeling in a dose dependent manner and eventually blocked the labeling with varying affinity (FIG. 18), suggesting a specificity. FSHR^{exo2} is the most potent inhibitor, suggesting the possibility of its strong interaction with the hormone. Furthermore, failed to label denatured FSH hat does not bind to the receptor, despite high concentrations of the peptide (FIG. 19A), suggesting the specificity of the affinity labeling for biologically active FSH. FSH was denatured by boiling in 8M urea for 30 minutes. To test whether the denatured FSH remained in solution, the mixture of radioactively labeled FSH and unlabeled FSH was denatured, and varying volumes of the mixture were transferred to another tubes and the radioactivity was counted. The transfer was quantitative with a 99-100 efficiency, indicating denatured FSH was present in the photoaffinity labeling tube. ¹²⁵I-ABG-FSHR^{exo3}I did not label urokinase, phospholipases A, C and D (FIG. 19B). In addition, it failed to noticeably label human growth hormone (FIG. 19B). The exoloop 3 peptide inhibited ¹²⁵I-FSH binding to the receptor on intact cells in a dose dependent manner. These results show that the peptide's binding to and labeling of FSH were specific to bioactive FSH. Since the a and b subunits of human FSH comigrate on SDS-PAGE, it is unclear which of the subunits was labeled. To determine the identity of the labeled subunit(s), FSH was labeled with ¹²⁵I-ABG-FSHR^{exo3}I, deglycosylated with PNGase F, and electrophoresed. The labeled band corresponded to the a subunit (FIG. 19B). Deglycosylated human FSH separates into two bands on SDS-PAGE, the higher molecular weight b subunit in the upper band and the smaller a subunit in the lower band, which was verified by monoclonal anti subunit antibodies.

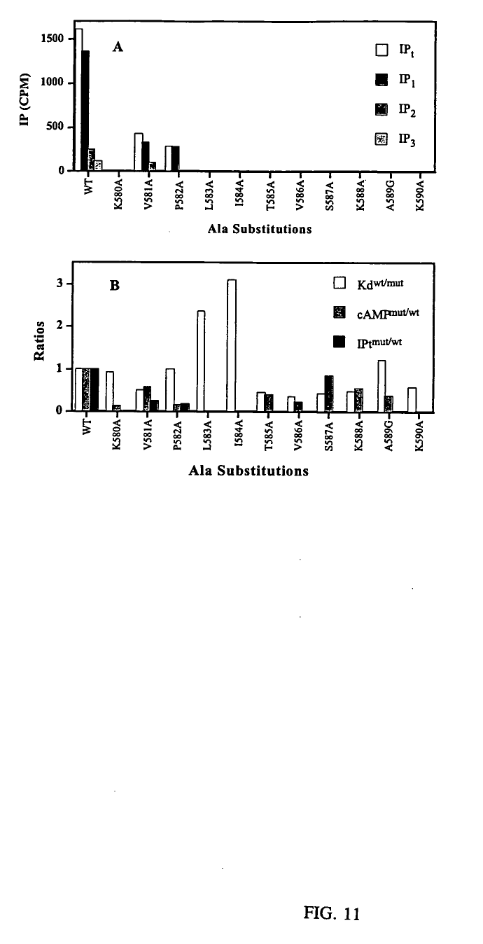

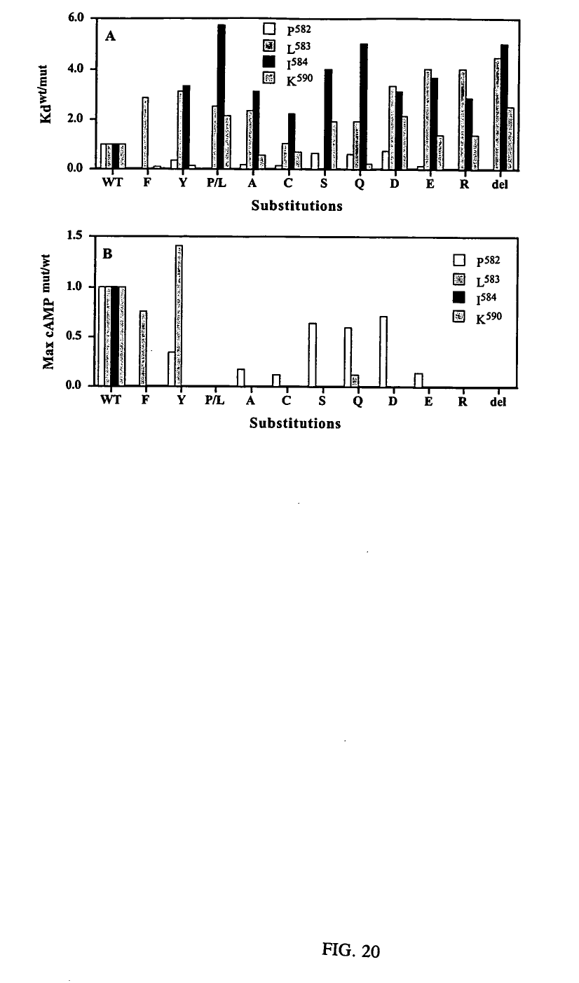

The results show that the exoloop 3 is crucially, yet differently, involved in hormone binding and induction of cAMP and IP. They show that FSHR exoloop 3 contacts the α subunit of FSH as part of the ternary complex consisting of FSH, the exodomain and endodomain. Particularly, L⁵⁸³ and I⁵⁸⁴ are more important than other amino acids, and project from one side of exoloop 3 in opposite directions. Interestingly, the substitutions of the two residues significantly improved the hormone binding, which is due to the loss of the original side chains rather than the introduction of the side chains from the substitutions. For example, all substitutions of I⁵⁸⁴ with Y, P, A, C, S, Q, D, E and R enhanced the hormone binding affinity, as did the deletion of I⁵⁸⁴ as shown by the Kd^{wt/mut} ratios in FIG. 11A. To analyze the nature of the effect, the side chain hydrophobicity of substituting amino acids was plotted against the Kd values (FIG. 20B). The plot showed two distinct groups, one consisting of ICAYQPS with a hydrophobicity/Kd coefficient of −1.03 and the other of PSEDR with a hydrophobic coefficient of 0.19. The first group had a negative hydrophobic effect on the binding affinity, whereas the second had a positive hydrophobic effect, suggesting a complex, specific microenvironment and interaction of the I⁵⁸⁴ side chain. The interaction appears to be partly hydrophobic and may involve other specificity such as stereo-specificity.

Substitutions of P⁵⁸² show diverse results, independent of the side chain's hydrophobicity. On the other hand, substitutions of K⁵⁹⁰ showed two distinct groups (FIG. 20B): the first group of FYQEDR with the most severe hydrophobicity/Kd coefficient of −8.1 and the second group of LCAPSEDR with a coefficient of −0.87. In the first group, ring groups such as the phenyl and phenolic side chains of F and Y, respectively, severely impaired the binding affinity, considerably more than any substitutions of L⁵⁸³ and I⁵⁸⁴ did. The second group also negatively impacted the binding affinity, but the effects were mild. A striking difference of the two groups is the substitutions with F and L. The Kd value of FSHR^{K590F} was 50 nM in contrast to 7.2 nM for FSHR^{K590L}, raising a question of whether the side chain flexibility and geometry play a role. The deletion of K⁵⁹⁰, thus, likely relieves the constraint and improves the Kd value as shown by the deletion mutant. All of the substitutions, except L⁵⁸³ Y, diversely impaired the cAMP induction (FIG. 20C). This adverse impact was seen the least on the substitutions of P⁵⁸². Among the various L⁵⁸³ mutants, only L⁵⁸³ F and L⁵⁸³Y, were capable of inducing cAMP production. This suggests that a hydrophobic side chain larger than a methyl group is necessary at the position, regardless of whether the side chain is aliphatic or aromatic. This is in contrast to the adverse effect of a ring group at K⁵⁹⁰ on the hormone binding, clearly indicating the requirements for distinct groups at L⁵⁸³ and K⁵⁹⁰. All other substitutions of L⁵⁸³ lead to insignificant cAMP induction. In addition, every substitution of I⁵⁸³ and K⁵⁹⁰ abolished cAMP, showing the irreplaceable nature of I⁵⁸⁴ and K⁵⁹⁰. The substitutions of K⁵⁹⁰ with A, C, F, L, Y and R abrogated IP induction, suggesting the irreplaceable role of K⁵⁹⁰ for both IP and cAMP induction. This is in contrast to the differential effects on cAMP and IP induction by Ala substitutions for the exoloop 3 amino acids, K⁵⁸⁰-K⁵⁹⁰.

In conclusion, these observations demonstrate the interaction of the FSHR exoloop 3 with FSH, specifically the α subunit.

TABLE 1
IPt production of L⁵⁸³ mutants
L⁵⁸³ was substituted with Y, F, A, E, R or deleted and the mutants
were expressed on HEK 293 cells. All of the mutants were expressed on
the cell surface and bound ¹²⁵I-FSH as described in FIG. 13. The assay
for total IP production in response to increasing concentrations of FSH
showed that only the L⁵⁸³ Y mutant was capable of producing IPt.

| | IPt (CPM) |
|---|---|
| L(wt) | 1,660 ± 240 |
| Y | 440 ± 17 |
| F | NS |
| A | NS |
| E | NS |
| R | NS |
| del | NS |

FSHR (human) KVPLITVSKAK
FSHR (Rat) - - -
FSHR (mouse) - - -
FSHR (bovine) - - - S-
FSHR (pig) S-
FSHR (sheep) - - - S-
FSHR (Horse) - - - S-
FSHR (chick) R - - - S-
FSHR (Equas)-A - - - S-
LHR (human) - - - TNS-
LHR (Rat) - - - TNS-
LHR (mouse) - - - TNS-
LHR (bovine) - - - TNS-
LHR (pig) - - - TNS-
LHR (sheep) - - - TNS-
LHR (carja)-M - - - TNS-
TSHR (human) NK - - - NS-
TSHR (Rat) NK - - - TNSG
TSHR (mouse) NK - - - TNS-
TSHR (bovine) NK - - - TNS-
TSHR (pig) NK - - - TNS-
TSHR (sheep) NK - - - TNS-
TSHR (Canfa) NK - - - TNS-

### EXAMPLE 3

Mutagenesis and Functional Expression of Human LH Receptor

Each mutant human LHR or FSHR cDNA was prepared in a pSELECT vector using the nonPCR based Altered Sites Mutagenesis System (Promega), sequenced on a Beckman CEQ 2000XL capillary sequencer, and subcloned into pcDNA3 (Invitrogen), as described. After subcloning pcDNA3, the mutant cDNAs were sequenced again. Plasmids were transfected into human embryonic kidney (HEK) 293 cells by the calcium phosphate method. Stable cell lines were established in minimum essential medium containing 8% horse serum and 500 mg/ml of G-418, and then used for hormone binding and cAMP assay. All assays were carried out in duplicate and repeated 4-6 times. Means and standard variations were calculated. In addition, values for mutants were compared with the corresponding values of the wild type receptor using ANOVA with 95% confidence to determine the statistical significance of differences as detailed in figure legends.

Hormone Binding and Intracellular cAMP Assay

hCG, human LH and human FSH were purchased from the National Hormone and Pituitary Program and radioiodinated as previously described. Stable cells were assayed for ¹²⁵I-hormone binding in the presence of increasing concentrations of nonradioactive hormone. The Kd values were determined by Scatchard plots. For intracellular cAMP assay, cells were washed twice with Dulbecco's modified Eagle's media and incubated in the media containing isobutylmethylxanthine (0.1 mg/ml) for 15 minutes. Increasing concentrations of hormone were then added and the incubation was continued for 45 minutes at 37° C. After removing the media, the cells were rinsed once with fresh media without isobutylmethylxanthine, lysed in 70% ethanol, freeze-thawed in liquid nitrogen, and scraped. After pelleting cell debris at 16,000×g for 10 minutes at 4° C., the supernatant was collected, dried under vacuum and resuspended in 10 ml of the cAMP assay buffer, which was provided by the manufacturer. cAMP concentrations were determined with an ¹²⁵I-cAMP assay kit (Amersham) following the manufacturer's instruction and validated for use in our laboratory.

Inositol Phosphate Assay

Stable cells were plated in 12 well plates and grown in inositol free DMEM (Atlanta Biologicals) supplemented with 8% heat-inactivated horse serum and 2 mCi/ml [³H]inositol (NEN) for 48 hours to 40-50% confluency. After removing the medium, the cells were incubated in 1 ml of fresh wash buffer consisting of DMEM w/o inositol and 15 mM HEPES (pH 7.3) for 1 hour at 37° C. This medium was removed and 0.3 ml wash buffer containing 20 mM LiCl was added and incubated for 15 minutes at 37° C. After the cells were stimulated with increasing concentrations of hormone for 30 minutes at 37° C., the incubation was terminated by the removal of medium and the addition of 0.25 ml of 0.6 N HCl to each well. The cells were scraped, transferred into microcentrifuge tubes and the wells were again washed with 0.25 ml of 0.6 N HCl. The combined washes were treated with 0.9 ml of a mixture of chloroform:methanol (2:1), vortexed and centrifuged at 1000×g for 5 minutes at room temperature. The top aqueous layer, which was free of phospholipids, was removed and the remaining chloroform layer treated with 0.2 ml of methanol:water (1:1), vortexed and centrifuged, as above. This aqueous layer was added to the previous aqueous layer and the samples dried in a vacuum concentrator. The dried samples were redissolved in 0.5 ml of 50 mM Tris-HCl, pH 8 and applied to Dowex AG 1-X8 formate (BIO-RAD) columns. The microcentrifuge tubes were washed twice with 0.5 ml of the same buffer and the washes applied to the columns for a total of 1.5 ml. The columns were sequentially washed with 4.5 ml H₂O and 4.5 ml 60 mM ammonium formate, 5 mM sodium tetraborate to elute the free inositol and the glycerol phosphoinositol. IP₁, IP2 and IP₃ were sequentially eluted with 4 ml of 0.1 N formic acid in 0.2 M, 0.75 M and 1.1 M ammonium formate, respectively, and collected in 1 ml fractions. Aliquots of 200 μl were counted for radioactivity in 1.5 ml of Ultima AF scintillation fluid (Packard). Peak radioactivities were used for the data analysis.

Derivatization and Radioiodination of Peptide

A peptide mimic corresponding to the LHR exoloop 3 sequence of K⁵⁷³ VPLITVTNSK⁵⁸³ (LHR^{exo3}) was synthesized, to which a Tyr residue was attached to the C-terminus for radioiodination. The N-terminus of the peptide was acetylated and the C-terminus amidated. NHS-ABG was synthesized as previously described and freshly dissolved in dimethyl sulfoxide to a concentration of 50 mM. The reagent was diluted in 0.1 M sodium phosphate (pH 7.5) to a concentration of 20 mM. The reagent solution was immediately used to derivatize receptor peptides. In the dark, 10 ml of NHS-ABG was added to 30 mg of receptor peptides in 40 ml of 0.1 M sodium phosphate (pH 7.5), and the mixture was incubated for 30 minutes at 25° C. The following were added to the derivatization mixture: 1 mCi of Na¹²⁵I-iodine in 10 μl of 0.1 M NaOH and 7 μl of chloramine-T (1 mg/ml) in 10 mM Na₂ HPO₄, pH 7.4. After 20 seconds, 7 μl of sodium metabisulfite (2.5 mg/ml) in 10 mM Na₂ HPO₄, pH 7.4, was introduced to terminate radioiodination. Derivatized and radioiodinated ABG-^{exo3} I-LHR^{exo3} solution was mixed with 60 μl of 16% sucrose solution in PBS and fractionated on Sephadex Superfine G-10 column (0.6×15 cm) using PBS.

Photoaffinity Labeling of hCG, Denatured hCG, LH and FSH

The following solutions were sequentially introduced to siliconized glass tubes and incubated: 20 μl of 0.9% NaCl and 10 mM Na₂ HPO₄, pH 7.4 (PBS), 100 ng/10 μl of hCG, denatured hCG, human LH or human FSH in PBS, and 10 ml of ABG-¹²⁵ I-LHR^{exo3} (10 ng/μl) in PBS. Competitive inhibition experiments were carried out as described for the photoaffinity labeling experiments, except that 10 μl, instead of 20 ml, of PBS was introduced to each tube and the mixture was incubated with 10 μl of increasing concentrations of nonradioactive receptor peptides. The mixtures were incubated at 37° C. for 90 minutes in the dark, irradiated with Mineralight R-52 UV lamp for 3 minutes as previously described, and solubilized in 2% SDS, 100 mM dithiothreitol and 8 M urea. The samples were electrophoresed on 8-12% polyacrylamide gradient gels. Gels were dried on filter paper and exposed to an imaging screen overnight, which was scanned on a phosphoimager. Exoloop 3 was modeled.

Effects of Ala Substitutions of Exoloop 3 Residues on hCG Dependent IP Induction

LHR exoloop 3 is known to constrain the hormone binding at the exodomain, but does not play a crucial role in cAMP induction except K⁵⁸³. To address this issue of the role of exoloop 3 in IP induction, individual Ala substituents of the exoloop 3 residues were stably expressed on HEK 293 cells and assayed for inositol phosphates, IP₁, IP₂, IP₃ and IPₜ. Most of the mutant receptors were incapable of inducing any of the IP species (FIGS. 1A-1H). However, the V⁵⁷⁴ A and S⁵⁸² A mutants responded to hCG and induced IP. The levels of IP₁, IP₂, IP₃ induced by the S⁵⁸² A mutant were similar to or slightly higher than the wild type levels, whereas the V⁵⁷⁴ A mutant induced detectable levels of IP₁, IP₂, and IP₃ that were considerably lower than the wild type levels. Therefore, the cells stably transfected with the mutants were assayed for hormone binding as well as hormone-dependent cAMP production.

Mutants were Differentially Impacted in IP and cAMP Induction and Hormone Binding

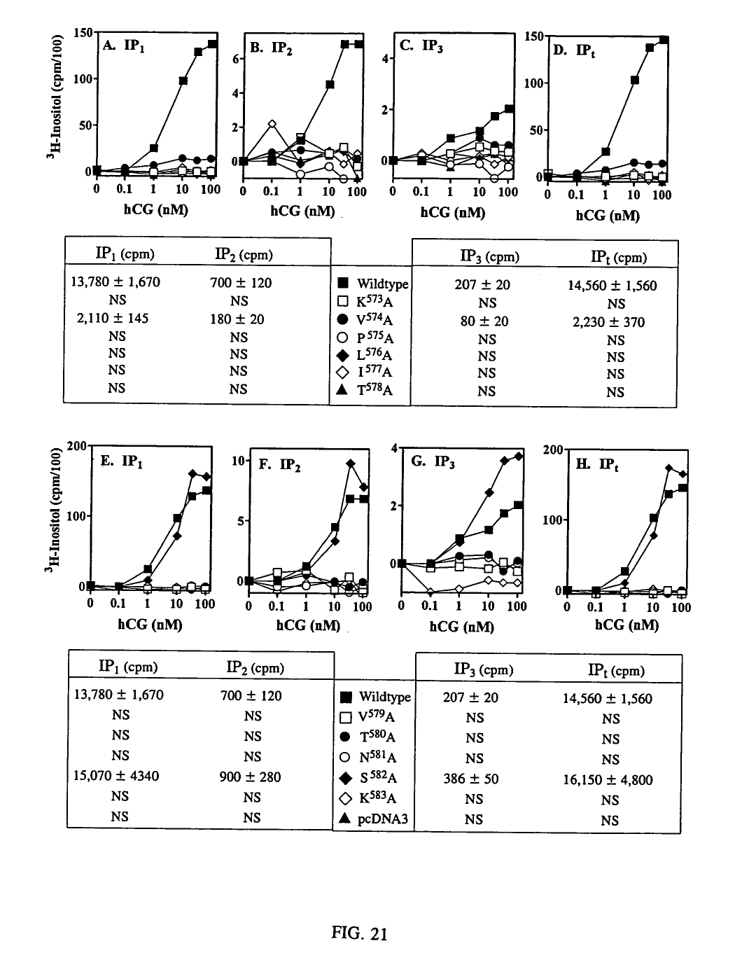

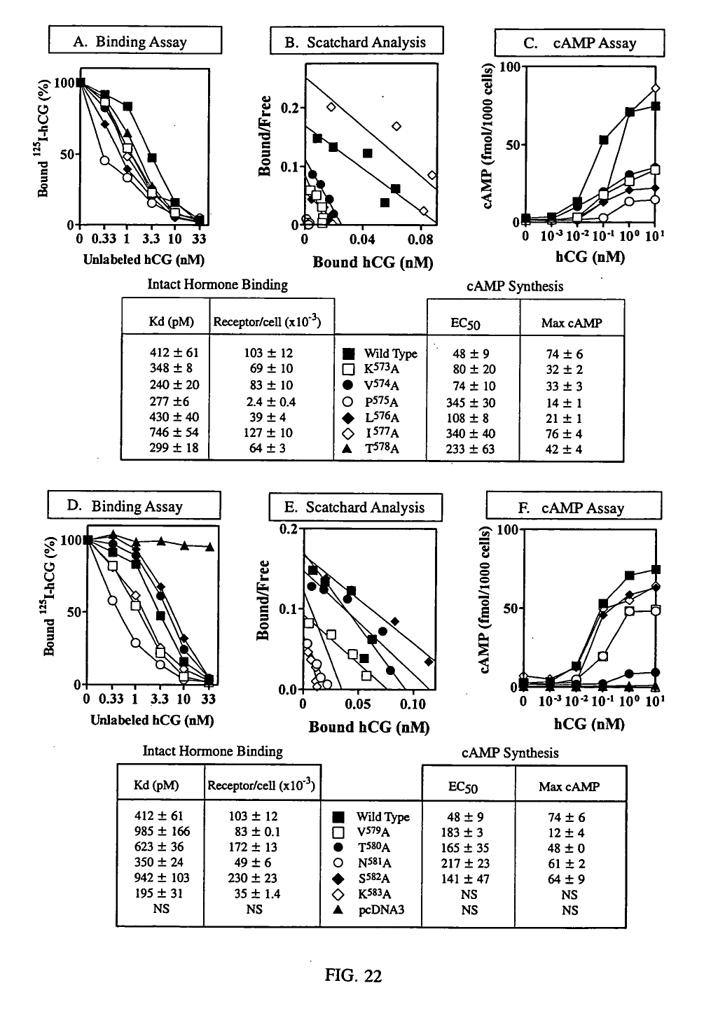

All of the mutants bound hCG and most of them, except LHR^{K583}, induced cAMP (FIG. 22), consistent with the previous report. Interestingly, the binding affinity of LHR^{K583A} was significantly better than the wild type receptor, yet incapable of inducing cAMP. This becomes more obvious when the ratios of Kd^{wild type/mutant} (Kd^{wt/mut}) maximum IPt^{mut/wt} and maximum cAMP^{mut/wt} were compared (FIG. 3). The ratios indicate that the mutant bound the hormone better and induced more cAMP and IP than the wild type receptor did. The summary underscores several features. The Ala substitutions differentially impacted hormone binding, cAMP induction and IP induction. IP induction was most sensitive to the Ala substitutions, whereas hormone binding was least sensitive. Furthermore, the substituted residues impacting the three functions were diverse, suggesting distinct mechanisms and residues involved in each of the three functions. For IP induction, all residues except V⁵⁷⁴ and S⁵⁸² appear to be crucial, whereas K⁵⁸³ and perhaps, a few others are essential for cAMP induction. Only, K⁵⁸³ is crucial for both IP and cAMP induction by LHR and FSHR. Because IP induction was most sensitive to the mutations, we were curious whether induction of IP₁, IP₂, and IP₃ in some of the mutants was differentially regulated. Therefore, the maximum IP ratios of mutant/wild type were examined for V⁵⁷⁴ A and S⁵⁸² A. The ratios for IP₁, IP₂, and IP₃ were 0.15, 0.25 and 0.39 for V⁵⁷⁴ A, and 1.09, 1.29 and 1.38 for S⁵⁸² A, suggesting variations. On the other hand, none of the three IP species was induced by the other mutants (FIG. 21).

Different Roles of Exoloop 3 in LHR and FSHR

The data indicate that LHR exoloop 3 is involved in IP induction, cAMP induction and hormone binding, but the mechanisms of these functions are distinct. Exoloop 3 is the shortest of the three exoloops, consisting of 11 amino acids, not only in LHR, but also in FSHR and the TSH receptor. The amino acid sequences are conserved among species except T⁵⁸⁰ N⁵⁸¹ of LHR and S⁵⁸⁷ K⁵⁸⁸ of FSHR. Considering the similarity of T and S, the homology between the gonadotropin receptors is high, which raises the issue of whether exoloop 3 of the two receptors work similarly or differently. To compare the functionality of these residues and effects of Ala substitutions in the two receptors, the ratios of Kd^{wt/mut}, cAMP^{mut/wt} and IP^{mut/wt} were examined. The differences between LHR and FSHR are clear. The K⁵⁸³ A substitution enhanced the binding affinity of LHR but the corresponding Ala substitution impaired that of FSHR. On the other hand, the converse was true with the I⁵⁷⁷ A substitution in LHR and the corresponding I⁵⁸⁴ A of FSHR. In addition, the I⁵⁸⁴ A substitution abolished cAMP induction of FSHR, but I⁵⁷⁷ A of LHR did not. The S⁵⁸² A substitution was the only one that did not impact on IP induction of LHR. Furthermore, it did not affect cAMP induction of LHR and not block hormone binding, suggesting that the S⁵⁸² A substitution is acceptable to LHR. It is interesting that FSHR has either A or S in place of S⁵⁸² of LHR, dependent on species. Yet, the substitution of G for A in FSHR completely abolished the IP induction. On the other hand, K⁵⁸³ A completely impaired both cAMP and IP induction by both receptors, although the substitution impacted differently on their binding affinity. Therefore, K⁵⁸³ of LHR and K 590 of FSHR were deleted or substituted with a panel of amino acids, A, D, E and R. The resulting mutant LHRs and FSHRs were capable of binding hCG and FSH, respectively, but there was no correlation between the Kd values of the corresponding LHR and FSHR mutant pairs.

Significant differences were observed in the exoloop 3 roles of FSHR and LHR in hormone binding, cAMP induction and IP induction. In addition, the last residues in exoloop 3, K⁵⁸³ of LHR and K⁵⁹⁰ of FSH, appear to play a similar role in IP and cAMP induction, but not in hormone binding, as do S⁵⁸² of LHR and A⁵⁸⁹ of FSHR. The differential roles of K⁵⁸³ of LHR and K⁵⁹⁰ of FSH in hormone binding are clearly shown by the multiple substitution and deletion studies. These results suggested the involvement of exoloop 3 in hormone binding.

Photoaffinity Labeling of hCG with Exoloop 3 Peptide Mimic

In an attempt to test whether LHR exoloop 3 interacts with hCG, the exoloop 3 peptide mimic, LHR^{exo3}, was synthesized and used for affinity labeling of hCG. The peptide was derivatized with a photoactivable reagent, the N-hydroxysuccinimide of 4-azidobenzoyl glycine (ABG) and radio-iodinated to produce ABG-¹²⁵ I-LHR^{exo3}. A constant amount of ABG-¹²⁵I-LHR^{exo3} was incubated with increasing concentrations of hCG and irradiated with UV. Samples were solubilized in SDS under the reducing condition and electrophoresed. The autoradiographic phosphoimage of the gel revealed the labeling of the hCG α and β bands with the a band labeled slightly more than the β band. The labeling increased in parallel to the concentration of hCG and then reached a plateau. The result suggests that the labeling was saturable at a certain hCG concentration and that additional hCG was not labeled. Furthermore, the maximum labeling was reached at 50 nM hCG for both of the hormone subunits, indicating that the slightly different labeling efficiencies of the subunits were independent of the hormone concentration. Next, increasing concentrations of ABG-¹²⁵I-LHR^{exo3} were incubated with a constant amount of hCG, photolyzed and processed as before. The resulting autoradiograph also shows the labeling of both of hCG α and β and a labeling plateau. In the next experiment, a constant amount of ABG-¹²⁵I-LHR^{exo3} was incubated with a constant concentration of hCG, and treated with UV for increasing time periods. The extent of the labeling increased as the UV photolysis time increased, plateauing at 90 seconds of irradiation. The hormone subunits were not labeled when the sample was not irradiated with UV, indicating that the labeling required UV photolysis. These results show that the labeling requires ABG-¹²⁵I-LHR^{exo3}, hCG and UV irradiation, and is saturable dependent on each of them. Both of the hCG α and β subunits were labeled, a slightly more than b. The extent of labeling of the α subunit and β subunit increased and plateaued in parallel throughout the hCG, peptide and UV dependent experiments. These results suggest that the hCG subunits and photoprobe were stably and specifically arranged in the ternary complex. In this spatial arrangement, the photoprobe is capable of labeling the α subunit slightly better than the β subunit, suggesting new insights into the geometry and proximity of the interacting exoloop 3 and the hCG subunits.

Specificity of Exoloop 3 Interaction

Although the labeling is saturable, its specificity was unclear. To test the biospecificity, ABG-¹²⁵I-LHR^{exo3} was incubated with increasing concentrations of denatured hCG and irradiated with UV. Denatured hCG was not labeled at all. To find out if higher concentrations of ABG-¹²⁵I-LHR^{exo3} were needed for labeling denatured hCG, increasing concentrations of ABG-¹²⁵I-LHR^{exo3} were incubated with a constant amount of denatured hCG. Higher concentrations of ABG-¹²⁵I-LHR^{exo3} failed to label denatured hCG. One may raise a concern of whether denatured hCG might have precipitated or adhered to the test tube during boiling in 8 M urea. To test whether Exoloop 3 of gonadotropin receptors denatured hCG remains in solution, unlabeled hCG was mixed with radioactively labeled hCG in 8 M urea and boiled for 30 minutes. Varying volumes of the mixture were transferred to another tubes, and the radioactivity was counted. The transfer was quantitative, indicating that denatured hCG remained in solution and was present in photoaffinity labeling tubes. The results demonstrate that the labeling requires bioactive hCG, not denatured hCG.

Thus, hCG and the exoloop 3 peptide should inhibit ¹²⁵I-hCG binding to LHR, but denatured hCG should not. To test the hypothesis, cells stably expressing LHR were incubated with ¹²⁵I-hCG in the presence of increasing concentrations of nonradioactive hCG, nonradioactive LHR^{exo3} or denatured hCG. The results show that both of the nonradioactive hCG and nonradioactive LHR^{exo3} inhibited ¹²⁵I-hCG binding to LHR, but hCG was >10,000 times more potent than the peptide mimic. However, denatured hCG failed to inhibit ¹²⁵I-hCG binding to LHR. hCG and LH bind to the same receptor and induce the similar hormone actions. Therefore, LHR^{exo3} is expected to similarly label both hormones. To test the possibility, increasing concentrations of LH and denatured LH were photoaffinity labeled with ABG-¹²⁵I-LHR^{exo3}. Both of the α and β subunits of LH were labeled, but denatured LH was not. Despite the biospecificity of photoaffinity labeling of hCG by LHR^{exo3}, it was unclear whether the derivatization of the peptide with ABG impacted on the peptide's specificity for hCG. To test the possibility, hCG was photoaffinity labeled with ABG-¹²⁵I-LHR^{exo3} in the presence of increasing concentrations of unlabeled LHR^{exo3} and scrambled LHR^{exo3}. The photoaffinity labeling was blocked by nonlabeled peptide blocked but not by nonlabeled scrambled peptide. In addition, phospholipase A, phospholipase C, phospholipase D and urokinase were incubated with ABG-¹²⁵I-LHR^{exo3} and photolyzed. These proteins were not photoaffinity labeled. These results show the specificity of the hCG photoaffinity labeling.

There were significant differences in the exoloop 3 roles, IP induction, cAMP induction and hormone binding, of LHR and FSHR. These are reflected in the 3 diverse amino acids, T/S-N/K-S/A, among the 11 residues in conserved exoloop 3 of the two receptors. There is also a remarkable difference in the photoaffinity labeling of hCG/LH and FSH with their respective exoloop 3 peptides. Whereas both subunits of hCG and LH were labeled by ABG-¹²⁵I-LHR^{exo3}), only the α subunit of FSH was labeled by ABG-¹²⁵I-FSHR^{exo3}. Since the α and β subunits of FSH comigrate on gel electrophoresis, photoaffinity labeled FSH was digested with PNGase F and electrophoresed, which resolves the two subunits. The labeled band of FSH corresponded to the α subunit, which is in contrast to the photoaffinity labeling of the FSH β subunit by the N-terminal peptide of the FSHR exodomain. Denatured FSH was not labeled at all. The photoaffinity labeling of the FSH α subunit is remarkable because there are two potential residues, K⁵⁸⁸ and K⁵⁹⁰, for ABG derivatization in FSHR^{exo3}, as compared to one derivatization site of K⁵⁸³ in LHRexo3. The results suggest notable differences in the structure and interaction of the exoloop 3s of LHR and FSHR. In fact, this view is consistent with the computer models of the two exoloops. The side chain orientation is particularly contrasting in the mid and C-terminal parts of the exoloop.

Relationship with Exoloops 1 and 2 and Other Regions of the Exodomain

hCG binds the exodomain with high affinity and three regions have been identified for the interaction. They are the N-terminal region, Leu Rich Repeat 4 and the hinge region. In addition, exoloop 2 is involved in the interaction of the exodomain and endodomain. The relationship between these various contact points likely plays a crucial role in the signal generation and therefore, the interaction of hCG and exoloop 3. Therefore, it is necessary to determine the relationship among the various interactions. hCG was photoaffinity labeled with ABG-¹²⁵I-LHR^{exo3} in the presence of 4 mM of nonlabeled exoloop peptides (LHR^{exo1}, LHR^{exo2} and LHR^{exo3}) N-terminal peptide (LHR¹⁸⁻³⁶), Leu Rich Repeat 4 peptide (LHR⁹⁶⁻¹¹⁵) and hinge region peptide (LHR²⁴⁶⁻²⁶⁹). Nonlabeled LHR^{exo3}, LHR⁹⁶⁻¹¹⁵ and LHR²⁴⁶⁻²⁶⁹ blocked the labeling (FIG. 13). LHR^{exo1} and LHR^{exo2} inhibited the labeling but the inhibition by LHR^{exo1} was considerably weak. These results suggest diverse affinities of the hCG labeling with these LHR peptides. On the other hand, LHR¹⁸⁻³⁶ failed to inhibit the labeling. These results clearly show the specificity of the hCG labeling by ABG-¹²⁵I-LHR^{exo3}.

The results show that LHR^{exo3} specifically photoaffinity labeled both subunits of hCG/LH, whereas the labeling of FSH by FHR^{exo3} was restricted to the α subunit of FSH. These gonadotropins share the common α0 subunits encoded in a single gene as well as the common hormone signals to activate AC for cAMP production and PLC β for production of IP and diacyl glycerol production. Because of these common structural feature and function, the common α subunit has been suspected to be involved in the hormone action. The results clearly support the possibility. The differential photoaffinity labeling of hCG/LH and FSH provides an explanation: the C-terminal region of exoloop 3 is in the proximity of both subunits of hCG/LH but the α subunit of FSH. This is consistent with the crystal structures of hCG and FSH. Although the overall structures are similar, there are differences in the β subunits that may be important with respect to receptor binding specificity or signal generation. For example, polar or charged residues in bloops, 3 (hFSH residues 62-73), the Cystine noose, determinant loop (residues 87-94) and the C-terminal loop (residues 94-104).

The labeling of hCG by ABG-¹²⁵I-LHR^{exo3} is by all three LHR exoloop peptides. However, their inhibition potency varies, the exoloop peptide 1 being the weakest. These results suggest the potential interactions of all three exoloops with hCG. This is consistent with the observation that the hinge of the exodomain constrains the AC activation in connection with exoloop 2. It will be interesting to see what the interaction and relationship of the three exoloops in their association with hCG. In fact, the labeling of hCG is blocked by the hinge peptide or LRR 4 peptide of the exodomain, but not by the N-terminal peptide. This selective labeling inhibition is surprising, because all of the three peptides were equally capable of labeling hCG. The labeling site of the exoloop3 peptide in hCG is different from the N-terminal peptide labeling site. On the other hand, it is unclear whether the exoloop3 labeling site is the same as or overlaps with the labeling sites of the Leu Rich Repeat 4 peptide and hinge peptide. These results provide new insights into the mode of the interactions among the exodomain, hCG and exoloop 3. For example, the initial, high affinity interaction between hCG and the exodomain involves all of the three sites. When the hCG/exodomain complex modulates the endodmain, it involves the contact between exoloop 3 and hCG at the site different from the contact site of the N-terminal peptide.

The endodomain is the site of signal generation, which likely involves all three exoloops. The data show that exoloop 3 plays roles in the activation of PLCβ/IP induction and activation of AC/cAMP induction as well as in the affinity of hormone binding. However, the importance of these roles is not equal: the PLCβ activation being most crucial and hormone binding least crucial. In fact, the role in the PLCβ activation is so crucial and most of the exoloop 3 residues appear to be involved. In contrast, there are fewer residues that appear crucial for the AC activation. They are P⁵⁷⁵, L⁵⁷⁶, V⁵⁷⁹ and K⁵⁸³. Substitution of these residues with Ala impaired the activation of PLCβ, AC or both. In addition, exoloop 3 constrains hormone binding at the exodomain, and Ala substitution for the residues often improved the binding affinity. In particular, the K⁵⁸³ A substitution resulted in 2 fold improvement in the binding affinity. These residues are not in tandem in a linear sequence, suggesting a spatial orientation or cluster.

The specific photoaffinity labeling of hCG by ABG-¹²⁵I-LHR^{exo3} shows the direct interaction, and this result is consistent with the inhibition of hCG binding to LHR by the peptide, albeit with low affinity. This is a novel and important observation and provides a new insight into the mechanism of the signal generation, particularly considering the recent reports that the exodomain modulates the signal generation by interacting with exoloop 2. Therefore, exoloop 3 interacts with the exodomain, in addition to the interaction with hCG, and participate in the signal generation. Nonlabeled exoloop 2 peptide blocked the labeling of both hCGα and hCGβ as did nonlabeled exoloop 3 peptide, suggesting the competitive nature of their interactions with hCG. Not only exoloop 2 but also the LRR 4 peptide, LHR⁹⁶⁻¹¹⁵ and the hinge region peptide, LHR²⁴⁶⁻²⁶⁹, inhibited the labeling. In contrast, the exoloop 1 peptide and the N-terminal peptide, LHR¹⁷⁻³⁶, were less potent in the inhibition. The results taken together show the specificity of the labeling and perhaps, interaction between hCG and these regions of LHR, which could provide new insights into the mechanistics of the interaction and signal generation.

In conclusion, the present invention presents the first evidence that LHR exoloop 3 interacts with hCG, and is involved in the differential activation of PLCβ and AC. Although FSHR exoloop 3 interacts with FSH and differentially modulates activation of PLCβ and AC, there are striking differences in the mode of the interactions and modulation between the two systems. LHR exoloop 3, in particular the C-terminal region of exoloop 3, is close to both of the hCG α and β subunits, whereas FSHR exoloop 3 is close to the FSH α subunit. In parallel to these distinct spatial arrangements, the tandem Leu-Ile sequence near the middle of exoloop3 is crucial for AC activation in FSHR but not in LHR. The penultimate C-terminal residue is essential for PLCα activation in LHR but not in FSHR. The interaction of exoloop 3 with hCG is related to the interactions of hCG with the hinge and LRR4 regions, but not the N-terminal region of the exodomain. This invention provides new insights into the transition from the initial interaction of hCG with the exodomain to the subsequent interaction with the exoloops, leading to signal generation.

TABLE II
Comparison of IP species by the wild type receptor and the V⁵⁷⁴A and
S⁵⁸² A mutants The maximum levels of IP species of LHR^{V574A} and
LHR^{S582A} were divided with the corresponding wild type values
presented in FIG. 1.

| | IP1 | IP2 | IP3 | IPt |
|---|---|---|---|---|
| V⁵⁷⁴A/wt | 0.15 | 0.25 | 0.39 | 0.15 |
| S⁵⁸²A/wt | 1.09 | 1.29 | 1.38 | 1.11 |

| IP₁ (cpm) | IP₂ (cpm) | | IP₃ (cpm) | IPₜ (cpm) |
|---|---|---|---|---|
| 13,780-w | 700 w 120 | L Wildtype | 207 w 20 | 14,560 w 1, [symbol] |
| 1,670 | | | | |
| NS | NS | I K⁵⁷³A | NS | NS |
| 2,110 w 145 | 180 w 20 | P V⁵⁷⁴A | 80 w 20 | 2,230 w 37 |
| NS | NS | p P⁵⁷⁵A | NS | NS |
| NS | NS | R L⁵⁷⁶A | NS | NS |
| NS | NS | r I⁵⁷⁷A | NS | NS |
| NS | NS | G T⁵⁷⁸A | NS | NS |
| 13,780-w | 700 w 120 | N Wildtype | 207 w 20 | 14,560 w 1, [symbol] |
| 1,670 | | | | |
| NS | NS | n V⁵⁷⁹A | NS | NS |
| NS | NS | P T⁵⁸⁰A | NS | NS |
| NS | NS | p N⁵⁸¹A | NS | NS |
| 15,070 w 4340 | 900 w 280 | R S⁵⁸²A | 386 w 50 | 16,150 w 4, [symbol] |
| NS | NS | r k⁵⁸³A | NS | NS |
| NS | NS | G pcDNA3 | NS | NS |

While this invention has been particularly shown and described with references to preferred embodiments thereof, it will be understood by those skilled in the art that various changes in form and details may be made therein without departing from the spirit and scope of the invention as defined by the appended claims.

All references discussed above are herein incorporated by reference in their entirety.

1. A method of modulating the interaction between CG and the LHR in a subject comprising administering a therapeutically effective amount of an agent which modulates a CG activity;
wherein the agent modulates CG activity by binding to exoloop 1, exoloop 2 or exoloop 3 of the LHR or to the binding domain of CG creating an agenvexodomain or agenVCG complex.

2. The method of claim 1, wherein the CG is hCG.

3. The method of claim 1, wherein the subject is a vertebrate or an invertebrate organism.

4. The method of claim 1, wherein the subject is a canine, a feline, an ovine, a primate, an equine, a porcine, a caprine, a camelid, an avian, a bovine, an amphibian, a fish, or a murine organism.

5. The method of claim 4, wherein the primate organism is a human.

6. The method of claim 5, wherein the human is male.

7. The method of claim 5, wherein the human is female.

8. The method of claim 1, wherein the agent is CG or a biologically active fragment thereof or other natural or synthetic compound.

9. The method of claim 1, wherein the agent blocks CG binding to the LHR by binding to the LHR.

10. The method of claim 1, wherein the agent blocks CG binding to the LHR by binding to the CG.

11. A method of regulating a CG activity in a subject comprising administering a therapeutically effective amount of an agent which modulates CG activity or modulates CG interaction with the LHR at the site of exoloop 1, exoloop 2 or exoloop 3 on the LHR by modulating the interaction of an exoloop 1, exoloop 2 or exoloop 3 motif on the LHR with the CG.

12. A method of treating a gonadotropin hormone related disease or condition in a male subject comprising administering to the subject a therapeutically effective amount of an agent which modulates CG activity or CG interaction with the LHR at the site of exoloop 1, exoloop 2 or exoloop 3 on the LHR.

13. The method of claim 12, wherein the gonadotropin hormone related disease or condition is selected from a group consisting of male pseudohermaphroditism, microphallus, gynecomastia, bilateral anorchia, absence of Leydig's cells, cryptorchidism, Noonan's syndrome and myotonic dystrophy, delayed puberty, precocious puberty, acne and impotence.

14. A method of treating a gonadotropin hormone related disease or condition in a female subject comprising administering to said subject a therapeutically effective amount of an agent which modulates CG activity or CG interaction with the LHR at the site of exoloop 1, exoloop 2 or exoloop 3 on the LHR.

15. The method of claim 14, wherein the gonadotropin hormone related disease or condition is selected from the group consisting of primary and secondary amenorrhea, delayed puberty, precocious puberty, endometriosis, acne, uterine myoma, ovarian and mammary cystic diseases, and breast and gynecological cancers.

16. A method of contraception in a subject comprising administering to a subject an amount of an agent effective at preventing conception,
wherein the agent inhibits CG activity or CG interaction with the exoloop 1, exoloop 2 or exoloop 3 domain of the LHR.

17. The method of claim 16, wherein the agent is CG, a biologically active fragment thereof or other synthetic or natural compound.

18. The method of claim 16, wherein the subject is female.

19. The method of claim 16, wherein the subject is male.

20. A method of promoting fertility in a subject comprising administering to a subject an amount of an agent effective at stimulating fertility,
wherein the agent stimulates CG activity or CG interaction with the exoloop 1, exoloop 2 or exoloop 3 domain of the LHR.

21. A method of screening for compounds which modulate the interaction between CG and the exoloop 1, exoloop 2 or exoloop 3 domain on the LHR comprising:
(a) attaching CG or a biologically active polypeptide fragment thereof to a substrate;
(b) exposing CG or the biologically active polypeptide fragment thereof to an agent; and
(c) determining whether said agent bound to CG or the biologically active polypeptide fragment thereof and further determining whether said agent modulates the interaction between CG and the exoloop 1, exoloop 2 or exoloop 3 domain of the LHR.

22. A compound identified by the method of claim 21.

23. A composition for treating gonadotropin hormone related diseases comprising a pharmaceutically effective amount of a compound which modulates the LHR at the exoloop 1, exoloop 2 or exoloop 3 domain and a pharmaceutically acceptable excipient.

24. The composition of claim 23, wherein the compound is CG or a biologically active fragment thereof or other natural or synthetic compound.

25. The composition of claim 23, wherein the LHR modulating compound is the compound of claim 22.

26. The composition of claim 23, wherein the LHR modulating compound is an agent which binds to CG thereby preventing its interaction with the LHR at the site of the exoloop 1, exoloop 2 or exoloop 3 on the LHR.

27. A composition for treating a gonadotropin hormone related disease comprising a pharmaceutically acceptable amount of an agent which modulates CG activity, wherein the agent is an antibody which binds to CG and thereby prevents CG from interacting with LHR at the exoloop 1, exoloop 2 or exoloop 3 domain.

28. A method of modulating at least one activity of CG comprising administering an effective amount of an agent which modulates at least one activity of CG at the exoloop 1, exoloop 2 or exoloop 3 domain of the LHR.

29. The method of claim 28, wherein the modulated activity is selected from the group consisting of stimulation of progesterone, androgen and estrogen and stimulation of development of the male and female gonads, follicles, placenta, maturation of oocytes and sperm and growth of cells and tissue.

30. A method of identifying binding partners for CG comprising the steps of:
(a) exposing the protein to a potential binding partner; and
(b) determining if an exoloop 1, exoloop 2 or exoloop 3 domain of the potential binding partner binds to CG.

31. A method of modulating the interaction between FSH and the FSHR in a subject comprising administering a therapeutically effective amount of an agent which modulates a FSH activity;
wherein the agent modulates the FSH activity by binding to the exoloop 1, exoloop 2 or exoloop 3 of the FSHR or to FSH creating an agent/exodomain or agent/FSH complex.

32. The method of claim 31, wherein the subject is a vertebrate or an invertebrate organism.

33. The method of claim 31, wherein the agent is FSH or a biologically active fragment thereof or other natural or synthetic compound.

34. The method of claim 31, wherein the agent blocks FSH binding to the FSHR by binding to the FSHR.

35. The method of claim 31, wherein the agent blocks FSH binding to the FSHR by binding to the FSH.

36. A method of regulating a FSH activity in a subject comprising administering a therapeutically effective amount of an agent which modulates FSH activity or modulates FSH interaction with the FSHR at the site of exoloop 1, exoloop 2 or exoloop 3 on the FSHR by modulating the interaction of a exoloop 1, exoloop 2 or exoloop 3 on the FSHR with the FSH.

37. A method of treating a gonadotropin hormone related disease or condition in a male subject comprising administering to the subject a therapeutically effective amount of an agent which modulates FSH activity or FSH interaction with the FSHR at the site of exoloop 1, exoloop 2 or exoloop 3 on the FSHR.

38. The method of claim 37, wherein the gonadotropin hormone related disease or condition is selected from a group consisting of male pseudohermaphroditism, microphallus, gynecomastia, bilateral anorchia, absence of Leydig's cells, cryptorchidism, Noonan's syndrome and myotonic dystrophy, delayed puberty, precocious puberty, acne and impotence.

39. A method of treating a gonadotropin hormone related disease or condition in a female subject comprising administering to said subject a therapeutically effective amount of which modulates FSH activity or FSH interaction at the site of exoloop 1, exoloop 2 or exoloop 3 on the FSHR.

40. The method of claim 39, wherein the gonadotropin hormone related disease or condition is selected from the group consisting of primary and secondary amenorrhea, delayed puberty, precocious puberty, endometriosis, acne, uterine myoma, ovarian and mammary cystic diseases, and breast and gynecological cancers.

41. A method of contraception in a subject comprising administering to a subject an amount of an agent effective at preventing conception,
wherein the agent inhibits FSH activity or FSH interaction with the exoloop 1, exoloop 2 or exoloop 3 domain of the FSHR.

42. The method of claim 41, wherein the agent is FSH, a biologically active fragment thereof or other synthetic or natural compound.

43. A method of promoting fertility in a subject comprising administering to a subject an amount of an agent effective at stimulating fertility,
wherein the agent stimulates FSH activity or FSH interaction with the exoloop 1, exoloop 2 or exoloop 3 domain of the FSHR.

44. A method of screening for compounds which modulate the interaction between FSH and the exoloop 1, exoloop 2 or exoloop 3 domain on the FSHR comprising:
(a) attaching FSH or a biologically active polypeptide fragment thereof to a substrate;
(b) exposing FSH or the biologically active polypeptide fragment thereof to an agent; and
(c) determining whether said agent bound to FSH or the biologically active polypeptide fragment thereof and further determining whether said agent modulates the interaction between FSH and the exoloop 1, exoloop 2 or exoloop 3 domain of the FSHR.

45. A compound identified by the method of claim 44.

46. A composition for treating gonadotropin hormone related diseases comprising a pharmaceutically effective amount of a compound which modulates the FSHR at the exoloop 1, exoloop 2 or exoloop 3 domain and a pharmaceutically acceptable excipient.

47. The composition of claim 46, wherein the compound which modulates the FSHR at the exoloop 1, exoloop 2 or exoloop 3 domain is FSH or a biologically active fragment thereof or other natural or synthetic compound.

48. The composition of claim 46, wherein the FSHR modulating compound is the compound of claim 45.

49. The composition of claim 46, wherein the FSHR modulating compound is an agent which binds to FSH thereby preventing its interaction with the FSHR at the site of exoloop 1, exoloop 2 or exoloop 3 on the FSHR.

50. A composition for treating a gonadotropin hormone related disease comprising a pharmaceutically acceptalbe amount of an agent which modulated FSH activity, wherein the agent is an antibody which binds to FSH and thereby prevents it from interacting with the FSHR at the exoloop 1, exoloop 2 or exoloop 3 domain.

51. A method of modulating at least one activity of FSH comprising administering an effective amount of an agent which modulates at least one activity of FSH at the exoloop 1, exoloop 2 or exoloop 3 domain of the FSHR.

52. The method of claim 51, wherein the modulated activity is selected from the group consisting of stimulation of progesterone, androgen and estrogen and stimulation of development of the male and female gonads, follicles, placenta, maturation of oocytes and sperm and growth of cells and tissue.

53. A method of identifying binding partners for FSH comprising the steps of:
(a) exposing the protein to a potential binding partner; and
(b) determining if the exoloop 1, exoloop 2 or exoloop 3 domain of the potential binding partner binds to FSH.
